(19) United States
(12) Patent Application Publication
(10) Pub. No.: US 2010/0210597 A1
(43) Pub. Date: Aug. 19, 2010

(54) C21-Deoxy Ansamycin Derivatives as Antitumor Agents

(76) Inventors: Steven Moss, Essex (GB); Christine Martin, Essex (GB); Ming Zhang, Essex (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/526,605

(22) PCT Filed: Mar. 3, 2008
(86) PCT No.: PCT/GB08/50150

§ 371 Date: Jan. 7, 2010

(30) Foreign Application Priority Data
Mar. 1, 2007 (GB) 0703973.8
Mar. 2, 2007 (GB) 0704088.4

(51) Int. Cl. A61K 31/675; C07D 225/06; A61K 31/33; A61P 35/00; A61P 31/00; A61P 25/00; A61P 37/06; A61P 9/00

(52) U.S. Cl. 514/81; 540/461; 514/183

(57) ABSTRACT

According to the invention there are provided derivatives of a C21-deoxy ansamycin or salt thereof which contain a 1-hydroxyphenyl moiety bearing at position 3 an aminocarboxy substituent, in which position 5 and the aminocarboxy substituent at position 3 are connected by an aliphatic chain of varying length characterised in that the 1-hydroxy position of the phenyl ring is derivatised by an aminoalkyleneaminocarbonyl group, which alkylene group (which may optionally be substituted by alkyl groups) has a chain length of 2 or 3 carbon atoms, a phosphoric acid, or a phosphoric acid ester (such as an alkyl ester) group, or a salt thereof, and which derivatising group increases the water solubility and/or the bioavailability of the parent molecule. Such compounds are useful in therapy eg in the treatment of cancer and B-cell malignancies.

### INTRODUCTION

The present invention relates to derivatives of C21-deoxy ansamycin compounds that are useful, e.g. in the treatment of cancer B-cell malignancies, malaria, fungal infection, diseases of the central nervous system and neurodegenerative diseases, diseases dependent on angiogenesis, autoimmune diseases or as a prophylactic pretreatment for cancer. In particular, the derivatives are pro-drugs of C21-deoxy ansamycin compounds and/or may be active in their own right. The present invention also provides methods for the production of these compounds and their use in medicine, in particular in the treatment and/or prophylaxis of cancer or B-cell malignancies.

### BACKGROUND OF THE INVENTION

The development of highly specific anticancer drugs with low toxicity and favourable pharmacokinetic characteristics comprises a major challenge in anticancer therapy.

The 90 kDa heat shock protein (Hsp90) is an abundant molecular chaperone involved in the folding and assembly of proteins, many of which are involved in signal transduction pathways (for reviews see Neckers, 2002; Sreedhar et al., 2004a; Wegele et al., 2004 and references therein). So far nearly 50 of these so-called client proteins have been identified and include steroid receptors, non-receptor tyrosine kinases e.g. src family, cyclin-dependent kinases e.g. cdk4 and cdk6, the cystic transmembrane regulator, nitric oxide synthase and others (Donzé and Picard, 1999; McLaughlin et al., 2002; Chiosis et al., 2004; Wegele et al., 2004; http://www.picard.ch/downloads/Hsp90interactors.pdf). Furthermore, Hsp90 plays a key role in stress response and protection of the cell against the effects of mutation (Bagatell and Whitesell, 2004; Chiosis et al., 2004). The function of Hsp90 is complicated and it involves the formation of dynamic multi-enzyme complexes (Bohen, 1998; Liu et al., 1999; Young et al., 2001; Takahashi et al., 2003; Sreedhar et al., 2004; Wegele et al., 2004). Hsp90 is a target for inhibitors (Fang et al., 1998; Liu et al., 1999; Blagosklonny, 2002; Neckers, 2003; Takahashi et al., 2003; Beliakoff and Whitesell, 2004; Wegele et al., 2004) resulting in degradation of client proteins, cell cycle dysregulation and apoptosis. More recently, Hsp90 has been identified as an important extracellular mediator for tumour invasion (Eustace et al., 2004). Hsp90 was identified as a new major therapeutic target for cancer therapy which is mirrored in the intense and detailed research about Hsp90 function (Blagosklonny et al., 1996; Neckers, 2002; Workman and Kaye, 2002; Beliakoff and Whitesell, 2004; Harris et al., 2004; Jez et al., 2003; Lee et al., 2004) and the development of high-throughput screening assays (Carreras et al., 2003; Rowlands et al., 2004). Hsp90 inhibitors include compound classes such as ansamycins, macrolides, purines, pyrazoles, coumarin antibiotics and others (for review see Bagatell and Whitesell, 2004; Chiosis et al., 2004 and references therein).

The benzenoid ansamycins are a broad class of chemical structures characterised by an aliphatic ring of varying length joined either side of an aromatic ring structure. Naturally occurring ansamycins include: macbecin and 18,21-dihydromacbecin (also known as macbecin I and macbecin II respectively) (1 & 2; Tanida et al., 1980), geldanamycin (3; DeBoer et al., 1970; DeBoer and Dietz, 1976; WO 03/106653 and references therein), and the herbimycin family (4; 5, 6, Omura et al., 1979, Iwai et al., 1980 and Shibata et al, 1986a, WO 03/106653 and references therein).

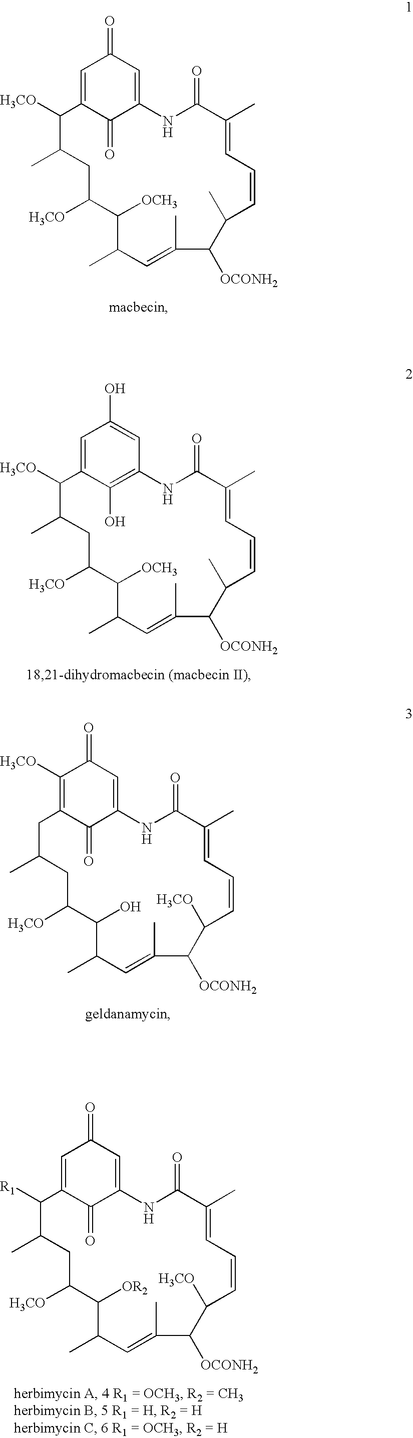

Ansamycins were originally identified for their antibacterial and antiviral activity, however, recently their potential utility as anticancer agents has become of greater interest (Beliakoff and Whitesell, 2004). Many Hsp90 inhibitors are currently being assessed in clinical trials (Csermely and Soti, 2003; Workman, 2003). In particular, geldanamycin has nanomolar potency and apparent specificity for aberrant protein kinase dependent tumour cells (Chiosis et al., 2003; Workman, 2003).

It has been shown that treatment with Hsp90 inhibitors enhances the induction of tumour cell death by radiation and increased cell killing abilities (e.g. breast cancer, chronic myeloid leukaemia and non-small cell lung cancer) by combination of Hsp90 inhibitors with cytotoxic agents has also been demonstrated (Neckers, 2002; Beliakoff and Whitesell, 2004). The potential for anti-angiogenic activity is also of interest: the Hsp90 client protein HIF-1α plays a key role in the progression of solid tumours (Hur et al., 2002; Workman and Kaye, 2002; Kaur et al., 2004).

Hsp90 inhibitors also function as immunosuppressants and are involved in the complement-induced lysis of several types of tumour cells after Hsp90 inhibition (Sreedhar et al., 2004). Treatment with Hsp90 inhibitors can also result in induced superoxide production (Sreedhar et al., 2004a) associated with immune cell-mediated lysis (Sreedhar et al., 2004). The use of Hsp90 inhibitors as potential anti-malaria drugs has also been discussed (Kumar et al., 2003). Furthermore, it has been shown that geldanamycin interferes with the formation of complex glycosylated mammalian prion protein PrP^{c} (Winklhofer et al., 2003).

As described above, ansamycins are of interest as potential anticancer and anti-B cell malignancy compounds, as well as having other potential utilities, however the currently available ansamycins exhibit poor pharmacological or pharmaceutical properties, for example they show poor water solubility, poor metabolic stability, poor bioavailability or poor formulation ability (Goetz et al., 2003; Workman 2003; Chiosis 2004). Both herbimycin A and geldanamycin were identified as poor candidates for clinical trials due to their strong hepatotoxicity (review Workman, 2003) and geldanamycin was withdrawn from Phase I clinical trials due to hepatotoxicity (Supko et al., 1995, WO 03/106653)

Geldanamycin was isolated from culture filtrates of Streptomyces hygroscopicus and shows strong activity in vitro against protozoa and weak activity against bacteria and fungi. In 1994 the association of geldanamycin with Hsp90 was shown (Whitesell et al., 1994). The biosynthetic gene cluster for geldanamycin was cloned and sequenced (Allen and Ritchie, 1994; Rascher et al., 2003; WO 03/106653). The DNA sequence is available under the NCBI accession number AY179507. The isolation of genetically engineered geldanamycin producer strains derived from S. hygroscopicus subsp. duamyceticus JCM4427 and the isolation of 4,5-dihydro-7-O-descarbamoyl-7-hydroxygeldanamycin and 4,5-dihydro-7-O-descarbamoyl-7-hydroxy-17-O-demethylgeldanamycin were described recently (Hong et al., 2004). By feeding geldanamycin to the herbimycin producing strain Streptomyces hygroscopicus AM-3672 the compounds 15-hydroxygeldanamycin, the tricyclic geldanamycin analogue KOSN-1633 and methyl-geldanamycinate were isolated (Hu et al., 2004). The two compounds 17-formyl-17-demethoxy-18-O-21-O-dihydrogeldanamycin and 17-hydroxymethyl-17-demethoxygeldanamycin were isolated from S. hygroscopicus NRRL 3602 containing plasmid pKOS279-78 with various genes from the herbimycin producing strain Streptomyces hygroscopicus AM-3672 (Hu et al., 2004). Genetic engineering of the geldanamycin biosynthetic pathway has led to the production of further geldanamycin analogues (Patel et al., 2004, Rascher et al., 2005) including non-benzoquinoid geldanamycin analogues, designated KOSN1559 and KOS-1806 which are phenolic. KOSN1559, a 2-desmethyl-4,5-dihydro-17-demethoxy-21-deoxy derivative of geldanamycin, binds to Hsp90 with a 4-fold greater binding affinity than geldanamycin and an 8-fold greater binding affinity than 17-AAG. However this was not reflected in an improvement in the IC₅₀ measurement using SKBr3. No activity data was given for KOS-1806.

In 1979 the ansamycin antibiotic herbimycin A was isolated from the fermentation broth of Streptomyces hygroscopicus strain No. AM-3672 and named according to its potent herbicidal activity. The antitumour activity was established by using cells of a rat kidney line infected with a temperature sensitive mutant of Rous sarcoma virus (RSV) for screening for drugs that reverted the transformed morphology of the these cells (for review see Uehara, 2003). Herbimycin A was postulated as acting primarily through the binding to Hsp90 chaperone proteins but the direct binding to the conserved cysteine residues and subsequent inactivation of kinases was also discussed (Uehara, 2003).

Chemical derivatives have been isolated and compounds with altered substituents at C19 of the benzoquinone nucleus and halogenated compounds in the ansa chain showed less toxicity and higher antitumour activities than herbimycin A (Omura et al., 1984; Shibata et al., 1986b). The sequence of the herbimycin biosynthetic gene cluster was identified in WO 03/106653 and in a recent paper (Rascher et al, 2005).

The ansamycin antibiotics macbecin (1) and 18,21-dihydromacbecin (2) (C-14919E-1 and C-14919E-1), identified by their antifungal and antiprotozoal activity, were isolated from the culture supernatants of Nocardia sp No. C-14919 (Actinosynnema pretiosum subsp pretiosum ATCC 31280) (Tanida et al., 1980; Muroi et al., 1980; Muroi et al., 1981; U.S. Pat. No. 4,315,989 and U.S. Pat. No. 4,187,292). 18,21-Dihydromacbecin is characterized by containing the hydroquinone form of the nucleus. Both macbecin and 18,21-dihydromacbecin were shown to possess similar antibacterial and antitumour activities against cancer cell lines such as the murine leukaemia P388 cell line (Ono et al., 1982). Reverse transcriptase and terminal deoxynucleotidyl transferase activities were not inhibited by macbecin (Ono et al., 1982). The Hsp90 inhibitory function of macbecin has been reported in the literature (Bohen, 1998; Liu et al., 1999). The conversion of macbecin and 18,21-dihydromacbecin after adding to a microbial culture broth into a compound with a hydroxy group instead of a methoxy group at a certain position or positions is described in U.S. Pat. No. 4,421,687 and U.S. Pat. No. 4,512,975.

During a screen of a large variety of soil microorganisms, the antibiotics TAN-420A to E were identified from producer strains belonging to the genus Streptomyces (7-11, EP 0 110 710).

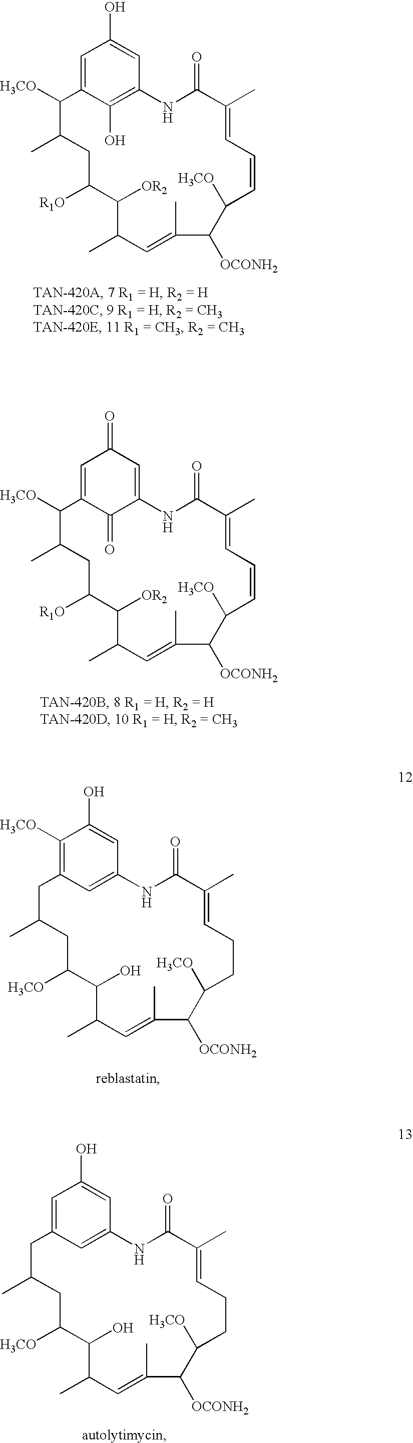

In 2000, the isolation of the geldanamycin related, non-benzoquinone ansamycin metabolite reblastatin (12) from cell cultures of Streptomyces sp. S6699 and its potential therapeutic value in the treatment of rheumatoid arthritis was described (Stead et al., 2000). Further naturally occurring non-quinone containing ansamycins have also been described such as autolytimycin (13, Stead et al 2000) which is the same as the engineered compound KOS-1806 (Rascher et al, 2005).

A further Hsp90 inhibitor, distinct from the chemically unrelated benzoquinone ansamycins is Radicicol (monorden) which was originally discovered for its antifungal activity from the fungus Monosporium bonorden (for review see Uehara, 2003) and the structure was found to be identical to the 14-membered macrolide isolated from Nectria radicicola. In addition to its antifungal, antibacterial, anti-protozoan and cytotoxic activity it was subsequently identified as an inhibitor of Hsp90 chaperone proteins (for review see Uehara, 2003; Schulte et al., 1999). The anti-angiogenic activity of radicicol (Hur et al., 2002) and semi-synthetic derivates thereof (Kurebayashi et al., 2001) has also been described.

Recent interest has focussed on 17-amino derivatives of geldanamycin as a new generation of ansamycin anticancer compounds (Bagatell and Whitesell, 2004), for example 17-(allylamino)-17-desmethoxy geldanamycin (17-AAG, 14) (Hostein et al., 2001; Neckers, 2002; Nimmanapalli et al., 2003; Vasilevskaya et al., 2003; Smith-Jones et al., 2004) and 17-desmethoxy-17-N,N-dimethylaminoethylamino-geldanamycin (17-DMAG, 15) (Egorin et al., 2002; Jez et al., 2003). More recently geldanamycin was derivatised on the 17-position to create 17-geldanamycin amides, carbamates, ureas and 17-arylgeldanamycin (Le Brazidec et al., 2003). A library of over sixty 17-alkylamino-17-demethoxygeldanamycin analogues has been reported and tested for their affinity for Hsp90 and water solubility (Tian et al., 2004). A further approach to reduce the toxicity of geldanamycin is the selective targeting and delivering of an active geldanamycin compound into malignant cells by conjugation to a tumour-targeting monoclonal antibody (Mander et al., 2000).

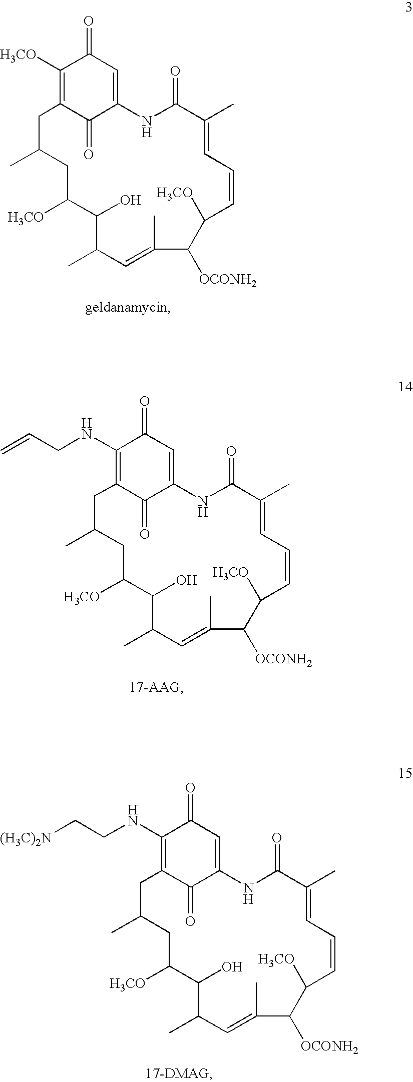

Whilst these derivatives exhibit reduced hepatotoxicity they still have only limited water solubility. For example 17-AAG (14) requires the use of a solubilising carrier (e.g. Cremophore®, DMSO-egg lecithin), which itself may result in side-effects in some patients (Hu et al., 2004).

Most of the ansamycin class of Hsp90 inhibitors bear a common structural moiety; the benzoquinone which is a Michael acceptor that can readily form covalent bonds with nucleophiles such as proteins, glutathione, etc. The benzoquinone moiety also undergoes redox equilibrium with dihydroquinone, during which oxygen radicals are formed, which give rise to further unspecific toxicity (Dikalov et al., 2002). For example treatment with geldanamycin can result in induced superoxide production (Sreedhar et al., 2004a). Therefore, there remains a need to identify novel ansamycin derivatives devoid of benzoquinone moiety, which may have utility in the treatment of cancer and/or B-cell malignancies, and other conditions, preferably such ansamycins have improved water solubility, an improved pharmacological profile and reduced side-effect profile for administration. The present invention discloses novel ansamycin derivatives which either have intrinsic activity or are pro-drugs of C21-deoxy ansamycins, such as compound 17, described and shown to be a potent Hsp90 inhibitor in WO2007/074347 (where it is described as compound 14). These compounds may be cleaved, chemically or enzymatically, to a C21-deoxy ansamycin. They will have improved pharmaceutical properties compared with the presently available ansamycins; in particular they show improvements in respect of one or more of the following properties: lower toxicity, higher water solubility, improved metabolic stability, bioavailability and formulation ability. Preferably the semi-synthetic derivatives of C21-deoxy ansamycin analogues show improved water solubility and/or bioavailability.

### SUMMARY OF THE INVENTION

The present invention provides derivatives of C21-deoxy ansamycins, methods for the preparation of these compounds, intermediates thereto and methods for the use of these compounds in medicine. In particular the derivatives of C21-deoxy ansamycins are pro-drugs and/or may be bioactive in their own right.

In its broadest aspect the present invention provides derivatives of C21-deoxy ansamycins which are derivatised at the phenolic position of the parent molecule. In at least some embodiments, these groups are designed to be self-cleaved or to cleave by enzymatic activity to produce the bioactive parent molecule. In other embodiments the compounds may be bioactive in themselves.

Thus the invention relates to derivatives of C21-deoxy ansamycins, or salts thereof, which contain a 1-hydroxyphenyl moiety bearing at position 3 an aminocarboxy substituent, in which position 5 and the aminocarboxy substituent at position 3 are connected by an aliphatic chain of varying length characterised in that the 1-hydroxy position of the phenyl ring is derivatised by an aminoalkyleneaminocarbonyl group, which alkylene group (which may optionally be substituted by alkyl eg methyl groups) has a chain length of 2 or 3 carbon atoms or a phosphoric acid, or a phosphoric acid ester (such as an alkyl ester) group, and which derivatising group increases the water solubility and/or the bioavailability of the parent molecule. Suitably the derivatising group is capable of being removed in vivo.

In this context the “parent molecule” means the corresponding molecule bearing an underivatised hydroxyl group at position 1 of the phenyl ring, corresponding to position 18 of the ansamycin.

In a more specific aspect the present invention provides derivatives of C21-deoxy ansamycins according to the formulas (IA-IC) below, or a pharmaceutically acceptable salt thereof:

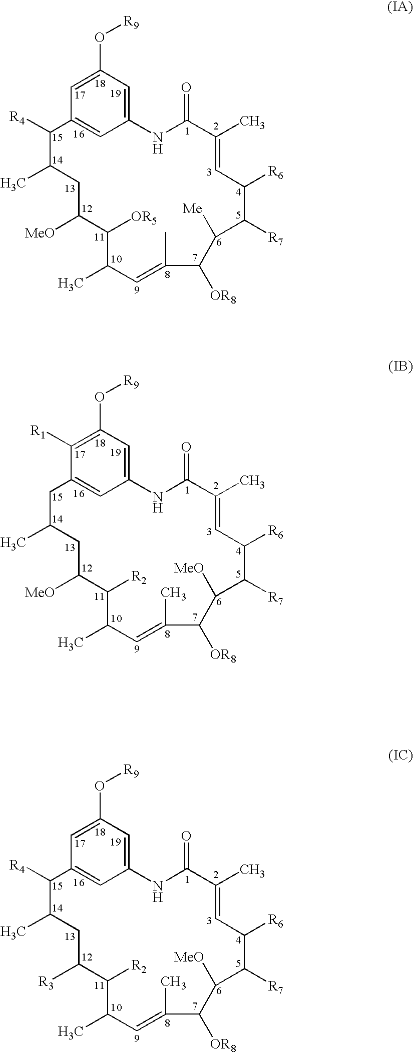

wherein:
R₁ represents H, OH, OMe;
R₂ represents OH, OMe or keto;
R₃ represents OH or OMe;
R₄ represents H, OH or OCH₃;
R₅ represents H or CH₃
R₆ and R₇ either both represent H or together they represent a bond (i.e. C4 to C5 is a double bond);
R₈ represents H or —C(O)—NH₂;
R₉ represents,

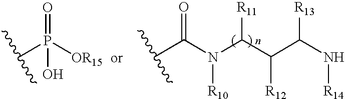

wherein:
   n represents 0 or 1;
   R₁₀ represents H, Me, Et or iso-propyl;
   R₁₁, R₁₂ and R₁₃ each independently represent H or a C1-C4 branched or linear chain alkyl group; or R₁₁ and R₁₂, or R₁₂ and R₁₃, may be connected so as to form a 6-membered carbocyclic ring;
   R₁₄ represents H or a C1-C4 branched or linear chain alkyl group; and
   R₁₅ represents H, Me or Et.

The above structures show a representative tautomer and the invention embraces all tautomers of the compounds of formula (IA), (IB) and (IC) for example keto compounds where enol compounds are illustrated and vice versa.

All stereoisomers of compounds of (IA), (IB) and (IC) (including all possible orientations of the phosphoric acid ester group) are embraced as an aspect of the invention.

Compounds of formula (IA), (IB) and (IC) are referred to collectively in the foregoing as compounds of formula (I).

In a further aspect, the present invention provides C21-deoxy ansamycin derivatives such as compounds of formula (I) or a pharmaceutically acceptable salt thereof, for use as a pharmaceutical.

### DEFINITIONS

The articles “a” and “an” are used herein to refer to one or to more than one (i.e. at least one) of the grammatical objects of the article. By way of example “an analogue” means one analogue or more than one analogue.

As used herein the term “analogue(s)” refers to chemical compounds that are structurally similar to another but which differ slightly in composition (as in the replacement of one atom by another or in the presence or absence of a particular functional group).

As used herein, the term “cancer” refers to a malignant new growth that arises from epithelium, found in skin or, more commonly, the lining of body organs, for example, breast, prostate, lung, kidney, pancreas, stomach or bowel. A cancer tends to infiltrate into adjacent tissue and spread (metastasise) to distant organs, for example to bone, liver, lung or the brain.

As used herein the term cancer includes both metastatic tumour cell types, such as but not limited to, melanoma, lymphoma, leukaemia, fibrosarcoma, rhabdomyosarcoma, and mastocytoma and types of tissue carcinoma, such as but not limited to, colorectal cancer, prostate cancer, small cell lung cancer and non-small cell lung cancer, breast cancer, pancreatic cancer, bladder cancer, renal cancer, gastric cancer, gliobastoma, primary liver cancer and ovarian cancer.

As used herein, the term “bioavailability” refers to the degree to which or rate at which a drug or other substance is absorbed or becomes available at the site of biological activity after administration. This property is dependent upon a number of factors including the solubility of the compound, rate of absorption in the gut, the extent of protein binding and metabolism etc. Various tests for bioavailability that would be familiar to a person of skill in the art are described herein (see also Egorin et al. (2002)).

As used herein the term “B-cell malignancies” includes a group of disorders that include chronic lymphocytic leukaemia (CLL), multiple myeloma, and non-Hodgkin's lymphoma (NHL). They are neoplastic diseases of the blood and blood forming organs. They cause bone marrow and immune system dysfunction, which renders the host highly susceptible to infection and bleeding.

The term “pro-drug” as used in this application refers to a precursor or derivative form of a pharmaceutically active substance that has an improved formulation profile compared to the parent drug, e.g. it may be less cytotoxic or more soluble compared to the parent drug, and it is capable of being activated (e.g. self-cleaved or enzymatically) or otherwise converted into the more active parent form (see, for example, Wilman D. E. V. (1986) “Pro-drugs in Cancer Chemotherapy” Biochemical Society Transactions 14, 375-382 (615th Meeting, Belfast) and Stella V. J. et al (1985) “Pro-drugs: A Chemical Approach to Targeted Drug Delivery” Directed Drug Delivery R. Borchardt et al (ed.) pages 247-267 (Humana Press).

The term “water solubility” as used in this application refers to solubility in aqueous media, e.g. phosphate buffered saline (PBS) at pH 7.4, or in 5% glucose solution. Tests for water solubility are given below in the Examples as “water solubility assay”.

As used herein, the term “C21-deoxy ansamycin derivative” refers to a benzenoid ansamycin derivative lacking the hydroxy group at position 21 referred to above as representing the invention in its broadest aspect, for example a compound according to formula (I) above, or a pharmaceutically acceptable salt thereof. These compounds are also referred to as “compounds of the invention” or “derivatives of C21-deoxy ansamycins” and these terms are used interchangeably in the present application.

The pharmaceutically acceptable salts of compounds of the invention such as the compounds of formula (I) include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Hydrochloric acid salts are of particular interest. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts. References hereinafter to a compound according to the invention include both compounds of formula (I) and their pharmaceutically acceptable salts. Alkyl, alkenyl and alkynyl groups may be straight chain or branched.

Examples of alkyl groups include C1-C4 alkyl groups such as methyl, ethyl, n-propyl, i-propyl and n-butyl. The expression “alkylene” may be interpreted in accordance with the term “alkyl”. Examples of alkenyl groups include C2-C4 alkyl groups such as ethenyl, n-propenyl, i-propenyl and n-butenyl.

As used herein the terms “18,21-dihydromacbecin” and “macbecin II” (the hydroquinone of macbecin I) are used interchangeably.

### FIGURE LEGEND

FIG. 1: Representation of the biosynthesis of macbecin showing the first putative enzyme free intermediate, pre-macbecin and the post-PKS processing to macbecin. The list of PKS processing steps in the figure is not intended to represent the order of events. The following abbreviations are used for particular genes in the cluster: AL0—AHBA loading domain; ACP—Acyl Carrier Protein; KS—β-ketoacylsynthase; AT—acyl transferase; DH—dehydratase; ER—enoyl reductase; KR—β-ketoreductase.

FIG. 2: Depiction of the sites of post-PKS processing of pre-macbecin to give macbecin.

FIG. 3: Diagrammatic representation of generation of the engineered strain BIOT-3806 in which plasmid pLSS308 was integrated into the chromosome by homologous recombination resulting in mbcM gene disruption.

FIG. 4: Diagrammatic representation of the construction of the in-frame deletion of mbcM described in example 4.

FIG. 5: Diagrammatic representation of the generation of an Actinosynnema pretiosum strain in which the mbcP, mbcP450, mbcMT1 and mbcMT2 genes have been deleted in frame following deletion of mbcM.

FIG. 6: A: In vitro conversion of 20 to 17 in human blood.

B: In vitro conversion of 20 to 17 in mouse blood.

FIG. 7: A: Pharmacokinetics of 17 in mouse plasma following oral administration of 20 at 10 mg/kg.

B: Pharmacokinetics of 20 and its metabolite 17 in mouse plasma following intravenous administration of 20 at 3 mg/kg.

FIG. 8: Chemical structures of compounds 20, 21, 22 and 23.

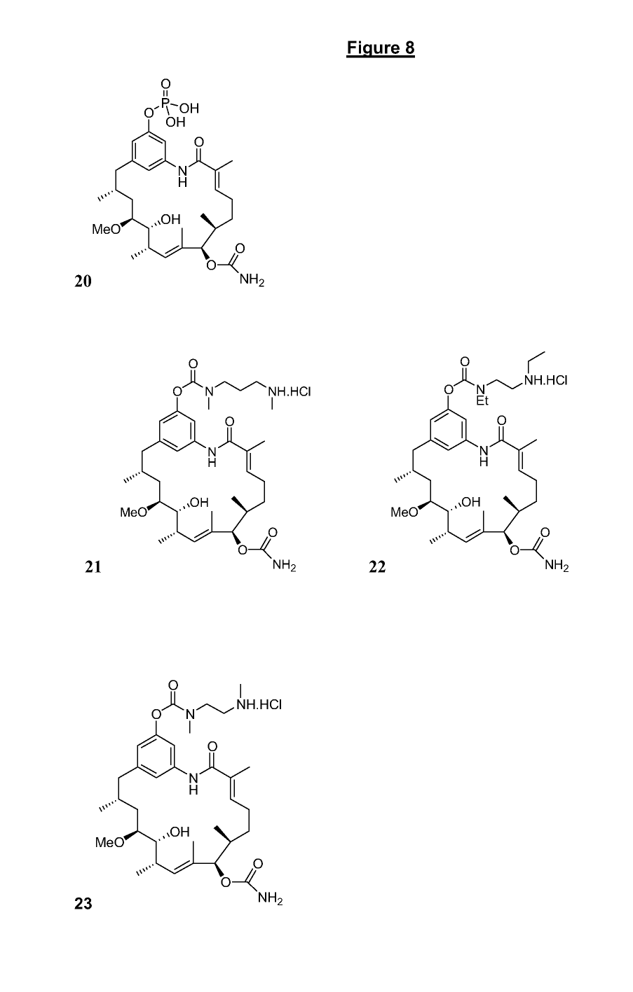

### DESCRIPTION OF THE INVENTION

The present invention provides a strategy to improve physical properties of C21-deoxy ansamycin drug candidates e.g. water solubility and/or bioavailability by employing a prodrug precursor. These pro-drugs may undergo enzymatic hydrolysis to release active parent drug or they may undergo self cleavage.

Without being limited by theory, in at least some embodiments, the present invention contemplates providing derivatives of C21-deoxy ansamycins with a method of active drug release. The active drug is released in at a rate that is controlled by the substrate structure. This approach should utilise self-cleavage of an incorporated amino side chain triggered at physiological pH via an intramolecular cyclisation-elimination reaction. Such intramolecular attack by a terminal amino group upon a carbamate functionality is expected to generate a cyclic urea fragment and thereby lead to parent drug release.

The rate of drug release should be governed by chemical cyclisation rate constants (and therefore pH) and associated substituents rather than by external influence.

Whilst it is expected that compounds of the invention that contain a carbamate group are capable of chemically mediated self-cleavage, it is also possible that they are substrates for enzymatic cleavage and this is also encompassed within the scope of the present invention. In an alternative embodiment the present invention provides derivatives of C21-deoxy ansamycins which are phosphate derivatives. These derivatives are believed to rely upon enzymatic hydrolysis to release active parent drug and/or may also be bioactive themselves. Thus, the present invention provides derivatives of C21-deoxy ansamycins, as set out above, methods for the preparation of these compounds, intermediates thereto and methods for the use of these compounds in medicine.

In one example set of compounds of formula IA-IC, R₁₀ represents Me or Et. In a further example set of compounds of formula IA-IC, R₁₄ represents a C1-4 branched or linear alkyl group. In a further example set of compounds of formula IA-IC, R₁₀ represents Me or Et and R₁₄ represents a C1-4 branched or linear alkyl group.

Suitably R₁ represents H, alternatively suitably R₁ represents OMe.
Suitably R₂ represents OH, alternatively suitably R₂ represents OMe.
Suitably R₃ represents OMe.
Suitably R₄ represents H. Alternatively suitably R₄ may represent OMe.
Suitably R₅ represents H.
Suitably R₆ represents H.
Suitably R₇ represents H.
Suitably R₈ represents —C(O)—NH₂.
Suitably R₉ represents,

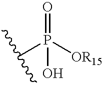

Suitably R₁₅ represents H. Alternatively R₁₅ represents Me or Et.

Suitably when R₉ represents the above mentioned phosphoric acid group or corresponding ester group, the compound of formula (I) may be provided as a mono or di basic salt eg with an alkali metal such as sodium.

Alternatively, suitably R₉ represents

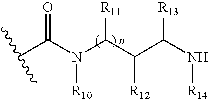

Suitably R₁₀ represents Me. Alternatively, suitably R₁₀ represents Et.
Suitably R₁₄ represents Me. Alternatively, suitably R₁₄ represents Et.
Suitably R₁₁ represents H. Suitably R₁₂ represents H. Suitably R₁₃ represents H.

When R₁₁ and R₁₂ or R₁₂ and R₁₃ are connected to form a six-membered carbocyclic ring that ring may suitably be a cyclohexyl ring.

Suitably n=0.

For example n may represent 0, R₁₂ and R₁₃ may each represent H and R₁₀ and R₁₄ may each represent Me. Alternatively n may represent 0, R₁₂ and R₁₃ may each represent H and R₁₀ and R₁₄ may each represent Et.

Alternatively n may represent 1, R₁₁, R₁₂ and R₁₃ may each represent H and R₁₀ and R₁₄ may each represent Me.

In one embodiment of the invention the compound is a compound of formula (IA). In another embodiment of the invention the compound is a compound of formula (IB). In another embodiment of the invention the compound is a compound of formula (IC).

In one embodiment of the invention suitably the compound is a C21-deoxy ansamycin of formula (IA) where R₄ represents H, R₅ represents H, R₆ represents H, R₇ represents H and R₈ represents —C(O)—NH₂.

Suitably the compound is a C21-deoxy ansamycin of formula (IA) where R₄ represents H, R₅ represents H, R₆ represents H, R₇ represents H, R₈ represents —C(O)—NH₂, R₉ represents

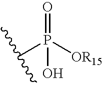

R₁₅ represents H, eg as represented by the following structure

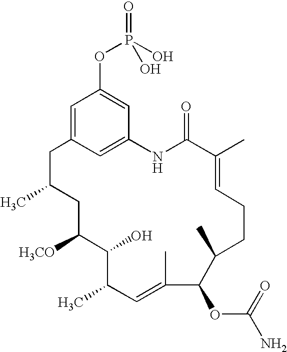

Suitably the compound is a mono-sodium salt of the C21-deoxy ansamycin of formula (IA) where R₄ represents H, R₅ represents H, R₆ represents H, R₇ represents H, R₈ represents —C(O)—NH₂, R₉ represents

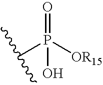

R₁₅ represents H, eg the sodium salt of this is represented by the following structure

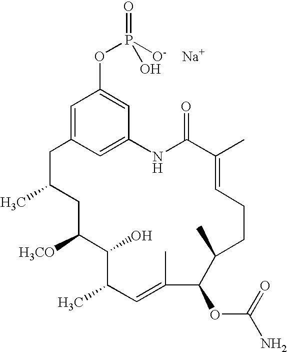

In another embodiment of the invention the compound is a C21-deoxy ansamycin of formula (IB) where R₁ represents H, R₂ represents OH, R₆ represents H, R₇ represents H and R₈ represents —C(O)—NH₂.

Suitably the compound is a C21-deoxy ansamycin of formula (IB) where R₁ represents H, R₂ represents OH, R₆ represents H, R₇ represents H, R₈ represents —C(O)—NH₂, R₉ represents

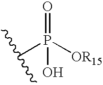

R₁₅ represents H, eg as represented by the following structure,

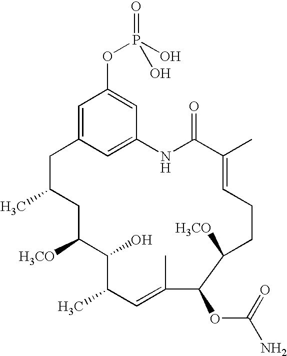

Suitably the compound is a mono sodium salt of a C21-deoxy ansamycin of formula (IB) where R₁ represents H, R₂ represents OH, R₆ represents H, R₇ represents H, R₈ represents —C(O) —NH₂, R₉ represents

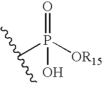

R₁₅ represents H, eg the sodium salt of this is represented by the following structure,

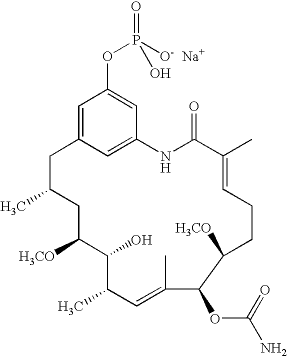

In another embodiment of the invention, the compound is a C21-deoxy ansamycin of formula (IB) where R₁ represents OMe, R₂ represents OH, R₆ represents H, R₇ represents H and R₈ represents —C(O)—NH₂.

Suitably the compound is a C21-deoxy ansamycin of formula (IB) where R₁ represents OMe, R₂ represents OH, R₆ represents H, R₇ represents H, R₈ represents —C(O)—NH₂, R₉ represents

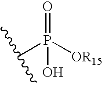

R₁₅ represents H, e.g. as represented by the following structure

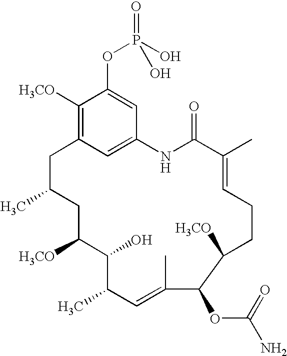

Suitably the compound is a mono sodium salt of a C21-deoxy ansamycin of formula (IB) where R₁ represents OMe, R₂ represents OH, R₆ represents H, R₇ represents H, R₈ represents —C(O)—NH₂, R₉ represents

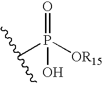

R₁₅ represents H, e.g. the sodium salt of this is represented by the following structure

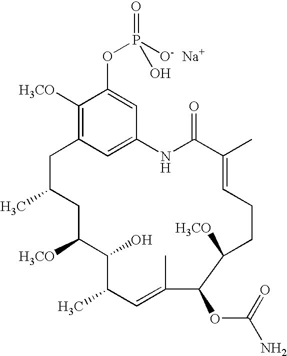

Alternatively suitably the compound is a C21-deoxy ansamycin of formula (IA) where R₄ represents H, R₅ represents H, R₆ represents H, R₇ represents H, R₈ represents —C(O)—NH₂, R₉ represents

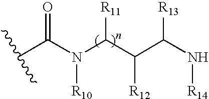

R₁₀ represents Me, R₁₂ represents H, R₁₃ represents H, R₁₄ represents Me and n=0 eg as represented by the following structure,

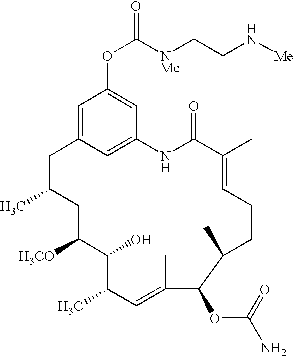

Suitably the compound is a C21-deoxy ansamycin of formula (IA) where R₄ represents H, R₅ represents H, R₆ represents H, R₇ represents H, R₈ represents —C(O)—NH₂, R₉ represents

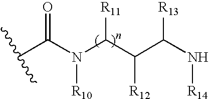

R₁₀ represents Et, R₁₂ represents H, R₁₃ represents H, R₁₄ represents Et and n=0 eg as represented in the following structure

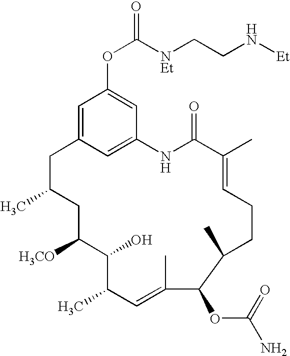

Suitably the compound is a C21-deoxy ansamycin of formula (IA) where R₄ represents H, R₅ represents H, R₆ represents H, R₇ represents H, R₈ represents —C(O)—NH₂, R₉ represents

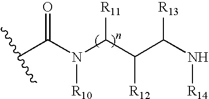

R₁₀ represents Me, R₁₁ represents H, R₁₂ represents H, R₁₃ represents H, R₁₄ represents Me and n=1 eg as represented by the following structure,

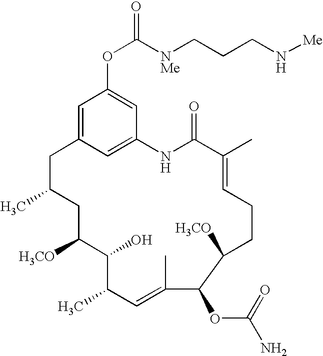

Suitably the compound is a C21-deoxy ansamycin of formula (IB) where R₁ represents H, R₂ represents OH, R₆ represents H, R₇ represents H and R₈ represents —C(O)—NH₂.

Suitably the compound is a C21-deoxy ansamycin of formula (IB) where R₁ represents H, R₂ represents OH, R₆ represents H, R₇ represents H, R₈ represents —C(O)—NH₂ and R₉ represents

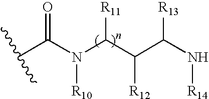

R₁₀ represents Me, R₁₂ represents H, R₁₃ represents H, R₁₄ represents Me and n=0 eg as represented by the following structure

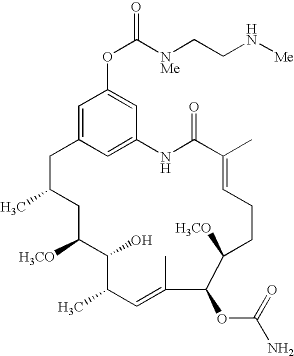

Suitably the compound is a C21-deoxy ansamycin of formula (IB) where R₁ represents H, R₂ represents OH, R₆ represents H, R₇ represents H, R₈ represents —C(O)—NH₂, R₉ represents

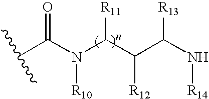

R₁₀ represents Me, R₁₁ represents H, R₁₂ represents H, R₁₃ represents H, R₁₄ represents Me and n=1, eg as represented in the following structure,

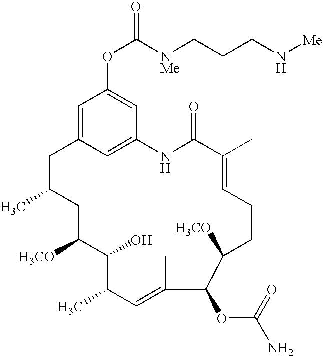

Suitably the compound is a C21-deoxy ansamycin of formula (IB) where R₁ represents H, R₂ represents OH, R₆ represents H, R₇ represents H, R₈ represents —C(O)—NH₂, R₉ represents

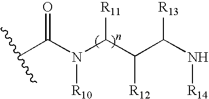

R₁₀ represents Et, R₁₂ represents H, R₁₃ represents H, R₁₄ represents Et and n=0 eg as represented by the following structure

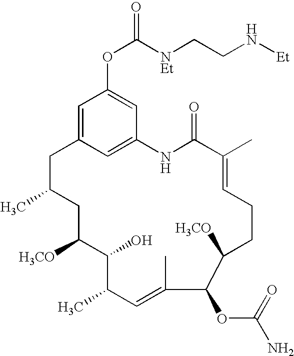

In another embodiment of the invention, the compound is a C21-deoxy ansamycin of formula (IB) where R₁ represents OMe, R₂ represents OH, R₆ represents H, R₇ represents H and R₈ represents —C(O)—NH₂.

Suitably the compound is a C21-deoxy ansamycin of formula (IB) where R₁ represents OMe, R₂ represents OH, R₆ represents H, R₇ represents H, R₈ represents —C(O)—NH₂ and R₉ represents

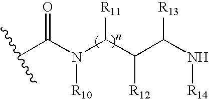

Suitably the compound is a C21-deoxy ansamycin of formula (IB) where R₁ represents OMe, R₂ represents OH, R₆ represents H, R₇ represents H, R₈ represents —C(O)—NH₂, R₉ represents

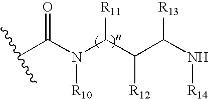

R₁₀ represents Me, R₁₂ represents H, R₁₃ represents H, R₁₄ represents Me and n=0 eg as represented by the following structure,

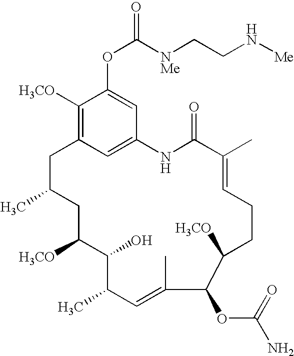

Suitably the compound is a C21-deoxy ansamycin of formula (IB) where R₁ represents OMe, R₂ represents OH, R₆ represents H, R₇ represents H, R₈ represents —C(O)—NH₂, R₉ represents

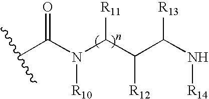

R₁₀ represents Me, R₁₁ represents H, R₁₂ represents H, R₁₃ represents H, R₁₄ represents Me and n=1 eg as represented by the following structure,

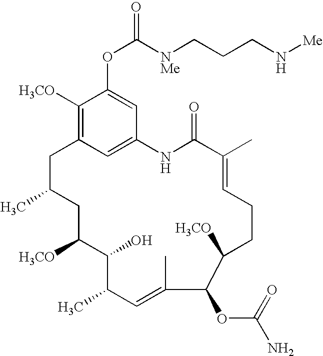

Suitably the compound is a C21-deoxy ansamycin of formula (IB) where R₁ represents OMe, R₂ represents OH, R₆ represents H, R₇ represents H, R₈ represents —C(O)—NH₂, R₉ represents

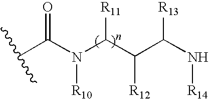

R₁₀ represents Et, R₁₂ represents H, R₁₃ represents H, R₁₄ represents Et and n=0 eg as represented by the following structure,

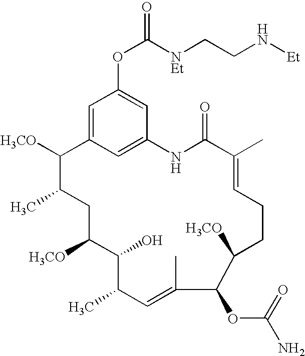

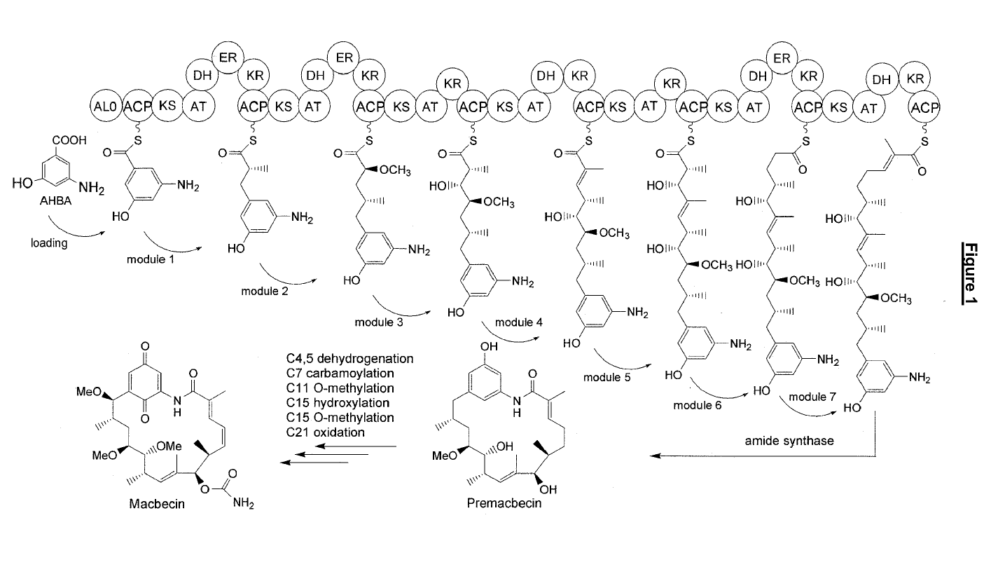

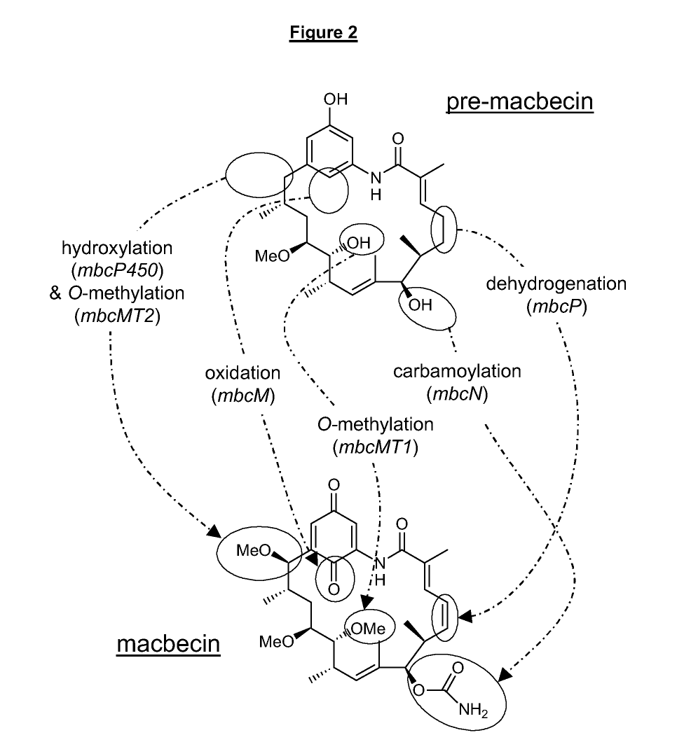

The stereochemistry of side chains relative to the ansamycin ring preferably follows that of naturally occurring ansamycin polyketides (i.e. macbecin—see FIGS. 1 and 2 below; geldanamycin; herbimycin A; reblastatin—see eg structure 17 in example 2 below).

The compound of formula (I) may, for example, represent a derivative of one of the following compounds:
C21-deoxymacbecin or an analogue (formula (IA) where R₉ represents H));
   Such as compound 17 as described in example 2, and shown to be a potent Hsp90 inhibitor in WO2007/074347 (where it is described as compound 14) (formula (IA) where R₄ represents H, R₅ represents H, R₆ represents H, R₇ represents H, R₈ represents C(O)NH₂, R₉ represents H)
C21-deoxy geldanamycin or an analogue (formula (IB) in which R₉ represents H);
C21-deoxy herbimycin A, B or C or an analogue (formula (IC) in which R₉ represents H);
Reblastatin or an analogue (formula (IB) in which R₂ represents OH, R₆ represents H, R₇ represents H, R₉ represents H).
   Such as reblastatin (12) (formula (IB) in which R₁ represents OMe, R₂ represents OH, R₆ represents H, R₇ represents H, R₈ represents C(O)NH₂, R₉ represents H).
Autolytimycin (13) (formula (IB) in which R₁ represents H, R₂ represents OH, R₆ represents H, R₇ represents H, R₈ represents C(O)NH₂ and R₉ represents H).

In general, the compounds of the invention are prepared by semi-synthetic derivatisation of C21-deoxy analogues of the ansamycin family of compounds.

A process for preparing a compound of formula (I) or a pharmaceutically acceptable salt thereof comprises:

(a) preparing a compound of formula (I) by reacting a compound of formula (IIA), (IIB) or (IIC):

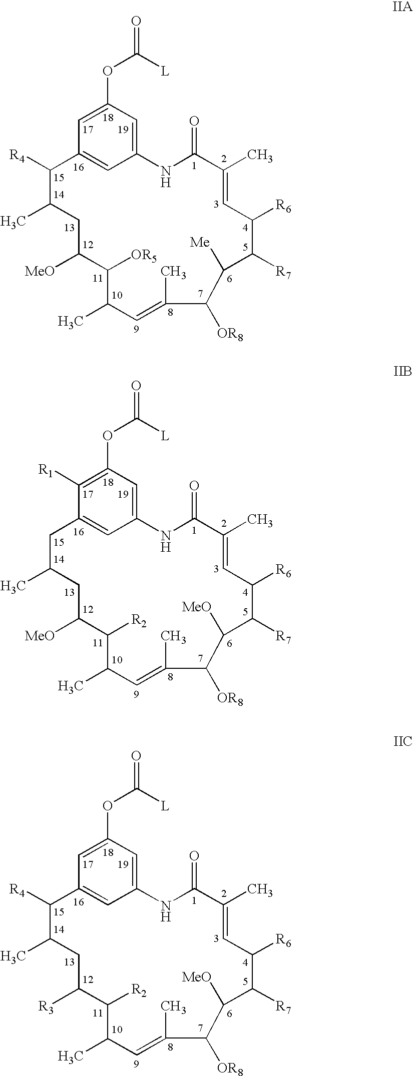

wherein L is a leaving group
or a protected derivative thereof, with a compound of formula (V)

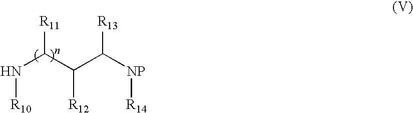

wherein P represents a protecting group; or
(b) preparing a compound of formula (I) by reacting a compound of formula (IIIA), (IIIB) or (IIIC)

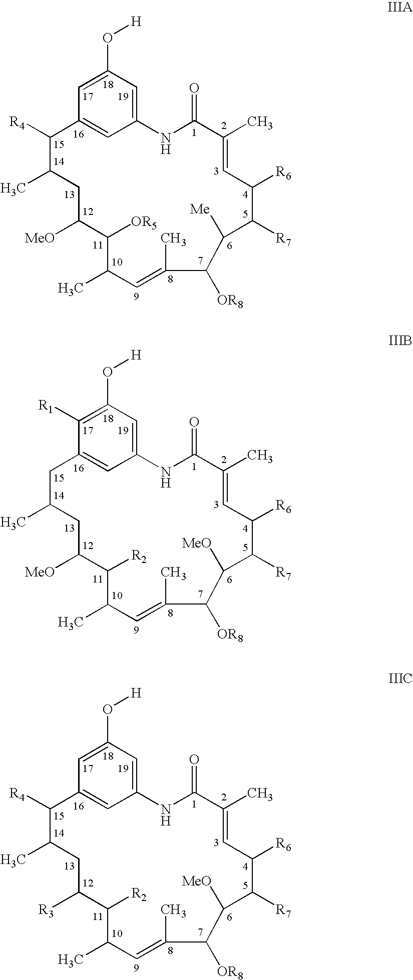

or a protected derivative thereof, with a phosphorylating reagent; or
(c) converting a compound of formula (I) or a salt thereof to another compound of formula (I) or another pharmaceutically acceptable salt thereof; or
(d) deprotecting a protected compound of formula (I).

In the foregoing text the compounds of formula (IIIA), (IIIB) and (IIIC), are referred to collectively as compounds of formula (III) and the compounds of formula (IIA), (IIB) and (IIC), are referred to collectively as compounds of formula (II).

In process (a) exemplary leaving groups L include halogen (eg chlorine, bromine), alkoxy (eg C1-4alkoxy), aryl (eg phenoxy or substituted phenoxy such as 4-nitrophenoxy) or alkylaryl (eg C1-4alkylaryl eg benzyloxy). Preferably L represents 4-nitrophenoxy. Exemplary protecting group P are those known to be suitable for amines, including substituted carbamates (e.g. BOC (i.e. t-butyloxycarbonyl) protecting group or TROC (i.e. 2,2,2-trichloroethoxycarbonyl) protecting group). Preferably the protecting group P is the trityl group.

The reaction of compounds of formula (II) with compound of formula (V) may be performed under conventional conditions known per se for carbamate formation eg reflux of the ingredients in an inert solvent such as dichloromethane.

Compounds of formula (II), or a protected derivative thereof, may be prepared by reacting a compound of formula IIIA-IIIC:

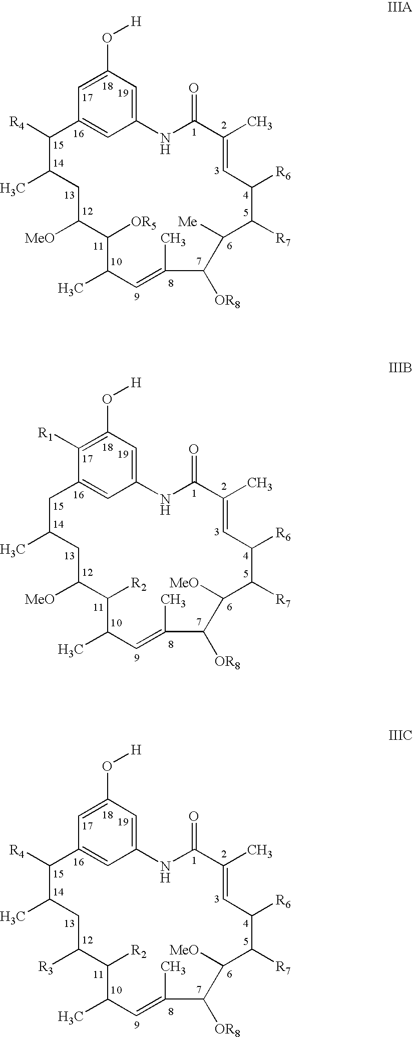

or a protected derivative thereof, with a compound of formula (J):

L′-CO-L (J)

wherein L′ represents a leaving group, preferably one which is more labile than L. Exemplary L′ groups are as described for L, above. A preferred compound of formula J is 4-nitrophenylchloroformate.

The reaction of compounds of formula (III) with compound of formula (J) may be performed under conventional conditions known per se eg reflux of the ingredients in an inert solvent such as dichloromethane with a slight excess of the compound of formula J and a suitable base.
Compounds of formula (V) may be produced by methods known to a person of skill in the art. For example, a suitable diamine can be selected and monoprotected eg using BOC, TROC or trityl group, most preferably the trityl group as a protecting group.

In addition to the specific methods and references provided herein a person of skill in the art may also consult standard textbook references for synthetic methods, including, but not limited to Vogel's Textbook of Practical Organic Chemistry (Furniss et al., 1989) and March's Advanced Organic Chemistry (Smith and March, 2001).

Compounds of formula (I), (II) and (III) may or do contain a secondary hydroxyl group at the C-11 position. In order to derivatise these compounds at the C-18 hydroxyl exclusively it may be necessary to first modify (i.e. protect) the C-11 hydroxyl. Described below are methods for accomplishing this for geldanamycin, but a person of skill in the art will appreciate that these can equally be applied to other compounds of formula (I), (II) and (III) for which the parent compound contains an OH group at C-11.

Protecting groups may, if desired, be generally employed in the synthesis of compounds of the invention and intermediate compounds as would be understood by a person skilled in the art.
The compounds of formula (III) can be reacted with a variety of phosphorylating derivatives that are in either a trivalent or pentavalent state.

When a compound of formula (III) is reacted with a trivalent phosphorylating reagent (e.g. phosphorus trichloride (phosphorus (III) chloride), or phosphamidates) then the resultant phosphite will require oxidation to the pentavalent phosphate with a suitable oxidising agent such as hydrogen peroxide or an organic peroxide.

The compounds of formula (III) can be reacted with pentavalent phosphorylating reagents, more generally provided by the definition P(═O)(OR₁₆)(OR₁₇)L

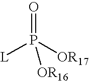

in which L represents a leaving group such as Cl and R₁₆ and R₁₇ are selected from alkyl, alkenyl, alkyl aryl or aryl groups such as benzyl, allyl, para-methoxybenzyl, which may be subsequently removed to provide the phosphoric acid group, by methods known to one skilled in the art, which includes treatment with acid, or hydrogenolysis.

The compounds of formula (III) can be reacted with pentavalent phosphorylating reagents, more generally provided by the definition P(═O)L₃

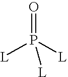

in which L represents a leaving group such as Cl. The product of this reaction can then be quenched, to remove the excess L groups, with alcohols or water.

An exemplary phosphorylating reagent is phosphoryl chloride which may be reacted with a compound of formula (III) dissolved in a suitable solvent and at a suitable temperature. It is also treated with a suitable base. Preferably the base is triethylamine or sodium hydride. Preferably the temperature is 30 degrees celcius or below, but not less than −78 degrees celcius and preferably not below −10 degrees celcius. A further nucleophile is added to the reaction mixture as described above, eg an alkyl or aryl alcohol or, more preferably, water. Phosphoric acid esters may be prepared from phosphoric acids by processes known to the skilled person.

With compounds of formula I, since R₉ is

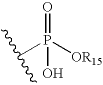

then various salts can be created. As is known to someone skilled in the art there are many methods for doing so, including the addition of a base such as XOH, XH or XOMe (where X=a singly charged cation) or YH₂ or Y(OH)₂ (where Y=a doubly charged cation). Alternatively the salt can be created on an ion exchange column. Preferably the sodium salt is created by passing the aqueous solution of such a compound through an ion exchange column, charged with Na⁺.

In addition to the specific methods and references provided herein a person of skill in the art may also consult standard textbook references for synthetic methods, including, but not limited to Vogel's Textbook of Practical Organic Chemistry (Furniss et al., 1989) and March's Advanced Organic Chemistry (Smith and March, 2001).

Compounds of formula (I), (II) and (III) may or do contain a secondary hydroxyl group at the C-11 position. In order to derivatise these compounds at the C-18 hydroxyl exclusively it may be necessary to first modify (i.e. protect) the C-11 hydroxyl. Described below are methods for accomplishing this for geldanamycin, but a person of skill in the art will appreciate that these can equally be applied to other compounds of formula (I), (II) and (III) for which the parent compound contains an OH group at C-11.

Protecting groups may, if desired, be generally employed in the synthesis of compounds of the invention and intermediate compounds as would be understood by a person skilled in the art. Other compounds embraced by the invention may be prepared by methods described herein and/or by methods known to a skilled person.

Salt formation and exchange may be performed by conventional methods known to a person of skill in the art. Interconversions of compounds of formula (I) may be performed by known processes for example hydroxy and keto groups may be interconverted by oxidation/reduction as described elsewhere herein.

Examples of protecting groups and the means for their removal can be found in T W Greene “Protective Groups in Organic Synthesis” (J Wiley and Sons, 1991). Suitable hydroxyl protecting groups include alkyl (e.g. methyl), acetal (e.g. acetonide) and acyl (e.g. acetyl or benzoyl) which may be removed by hydrolysis, and arylalkyl (e.g. benzyl) which may be removed by catalytic hydrogenolysis, or silyl ether, which may be removed by acidic hydrolysis or fluoride ion assisted cleavage. Suitable amine protecting groups include sulphonyl (e.g. tosyl), acyl (e.g. benzyloxycarbonyl or t-butoxycarbonyl) and arylalkyl (e.g. benzyl) which may be removed by hydrolysis or hydrogenolysis as appropriate.

Other compounds of the invention may be prepared by methods known per se or by methods analogous to those described above.

Compounds of formula IIIA-IIIC (hereinafter “compounds of formula (III)”) and protected derivatives thereof may be prepared as follows:

Firstly, the naturally occurring C21-deoxy ansamycins for use as templates may be obtained via direct fermentation of strains which produce the desired compound. A person of skill in the art will be able to culture a producer strain under suitable conditions for the production and isolation of the natural product template. The strains listed in Table 1 are examples of producer strains for the natural product templates, but a person of skill in the art will appreciate that there may be alternative strains available that will produce the same compound under appropriate conditions. A person skilled in the art will also appreciate that there may be strains that produce other natural product templates that are useful in this invention.

TABLE 1
producer strains

| Natural Product Template | Producer strain(s) |
|---|---|
| Reblastatin | Streptomyces sp. S6699 (Stead et al 2000) |
| Lebstatin | Streptomyces sp. S6699 (Stead et al 2000) |
| Autolytimycin | Streptomyces sp. S6699 (Stead et al 2000) |

Alternatively, the natural product compounds that can be used as templates may become commercially available.

Alternatively, C21-deoxy ansamycin templates may be generated by genetic engineering of the appropriate biosynthetic pathway. Published methods for making such compounds are listed in Table 2

TABLE 2
C21-deoxy ansamycin templates generated by genetic engineering
as described in literature

| Engineered Product Template | Producer strain(s) |
|---|---|
| KOS-1806 | S. hygroscopicus ‘gdmM-null mutant’ |
| | Rascher et al 2005 |
| KOSN1559 | S. hygroscopicus K309-1 Patel et al 2004 |

Alternatively, C21-deoxy ansamycin templates may be generated by employing genetic engineering methods such as those used to generate the compounds listed in Table 2, or those described herein, to the biosynthetic pathway of an ansamycin polyketide such as those listed in Table 3.

TABLE 3
producer strains of ansamycin polyketides, the biosynthetic
pathways governing the biosynthesis of these compounds can
be engineered to provide the C21-deoxy ansamycin
templates used in this invention.

| Ansamycin polyketide | Producer strain(s) |
|---|---|
| Macbecin and 18,21- | Actinosynnema pretiosum subsp pretiosum |
| dihydromacbecin | ATCC31280 |
| | Actinosynnema mirum DSM43827 |
| Herbimycin A-C | Streptomyces. hygroscopicus AM-3672 |
| Geldanamycin | Streptomyces. hygroscopicus var |
| | geldanus NRRL 3602 |
| | Streptomyces violaceusniger DSM40699 |
| | Streptomyces sp. DSM4137 |
| TAN 420A-E | Streptomyces. hygroscopicus AM-3672 |
| Reblastatin | Streptomyces sp. S6699 (Stead et al 2000) |
| Autolytimycin | Streptomyces sp. S6699 (Stead et al 2000) |
| Lebstatin | Streptomyces sp. S6699 (Stead et al 2000) |

The strains listed in Table 3 are examples of producer strains for the natural occurring ansamycins, but a person of skill in the art will appreciate that there may be alternative strains available that will produce the same compound under appropriate conditions.
A comprehensive description of how such engineering may be achieved follows below and is enabled in examples 1, 2 and 3. It is not a pre-requirement to have sequence information covering the biosynthesis of such a cluster, although acquiring this is routine and an example of how this can be achieved for the macbecin cluster is described in example 1. Furthermore, it is not a requirement to obtain a cluster sequence in order to carry out genetic manipulations to deliver the templates. For example, traditional methods of mutagenesis followed by screening may provide the compounds or, as described in example 2, one skilled in the art will be able to use publicly available sequences of homologous genes in order to engineer a pathway without first obtaining any sequence of the cluster to be manipulated. It is however advantageous to have the sequence of the cluster and example 3 describes how the sequence generated by the methods described in example 1 is used to generate compound 17. The cluster sequences that are available in the public domain that can be used to generate C21-deoxy ansamycin templates for derivatisation are given in Table 4.

TABLE 4
sequences of ansamycin polyketides.

| Ansamycin polyketide | Source of sequence data |
|---|---|
| Macbecin and 18,21- | Example 1 herein |
| dihydromacbecin | |
| Herbimycin A-C | Accession number AY947889 |
| Geldanamycin | Accession number AY179507 |

The inventors of the present invention have made significant effort to clone and elucidate the gene cluster that is responsible for the biosynthesis of macbecin. With this insight, the gene that is responsible for the production of the benzoquinone moiety has been specifically targeted in order to generate C21-deoxymacbecin analogues, e.g. by integration into mbcM, targeted deletion of a region of the macbecin cluster including all or part of the mbcM gene optionally followed by insertion of gene(s). Other methods of rendering MbcM non-functional include chemical inhibition, site-directed mutagenesis or mutagenesis of the cell for example by UV, in order to produce C21-deoxy analogues. Optionally targeted inactivation or deletion of further genes responsible for the post-PKS modifications of macbecin may be carried out to generate templates for derivatisation. Additionally, some of these genes, but not mbcM may be re-introduced into the cell. The optional targeting of the post-PKS genes may occur via a variety of mechanisms, e.g. by integration, targeted deletion of a region of the macbecin cluster including all or some of the post-PKS genes optionally followed by insertion of gene(s) or other methods of rendering the post-PKS genes or their encoded enzymes non-functional e.g. chemical inhibition, site-directed mutagenesis or mutagenesis of the cell for example by the use of UV radiation.

Where the compounds of the invention may be enzymatically or chemically cleaved, cleavage assays to assess the rate of cleavage are known in the art and are described below.
The above structures of intermediates may be subject to tautomerization and where a representative tautomer is illustrated it will be understood that and all tautomers for example keto compounds where enol compounds are illustrated and vice versa are intended to be referred to.
Novel compounds of formula (II) and (III), and protected derivatives thereof are also claimed as an aspect of the invention.

In one aspect the present invention provides for the use of a C21-deoxy ansamycin derivative in the manufacture of a medicament. In a further embodiment the present invention provides for the use of a C21-deoxy ansamycin derivative in the manufacture of a medicament for the treatment of cancer and/or B-cell malignancies. In a further embodiment the present invention provides for the use of a C21-deoxy ansamycin derivative in the manufacture of a medicament for the treatment of malaria, fungal infection, diseases of the central nervous system, diseases dependent on angiogenesis, autoimmune diseases and/or as a prophylactic pretreatment for cancer.

In another aspect the present invention provides for the use of a C21-deoxy ansamycin derivative in medicine. In a further embodiment the present invention provides for the use of a C21-deoxy ansamycin derivative in the treatment of cancer and/or B-cell malignancies. In a further embodiment the present invention provides for the use of a C21-deoxy ansamycin derivative in the manufacture of a medicament for the treatment of malaria, fungal infection, diseases of the central nervous system and neurodegenerative diseases, diseases dependent on angiogenesis, autoimmune diseases and/or as a prophylactic pretreatment for cancer.

In a further embodiment the present invention provides a method of treatment of cancer and/or B-cell malignancies, said method comprising administering to a patient in need thereof a therapeutically effective amount of a C21-deoxy ansamycin derivative. In a further embodiment the present invention provides a method of treatment of malaria, fungal infection, diseases of the central nervous system and neurodegenerative diseases, diseases dependent on angiogenesis, autoimmune diseases and/or a prophylactic pretreatment for cancer, said method comprising administering to a patient in need thereof a therapeutically effective amount of a C21-deoxy ansamycin analogue.

As noted above, compounds of the invention may be expected to be useful in the treatment of cancer and/or B-cell malignancies. Compounds of the invention and especially those which may have good selectivity for Hsp90 and/or a good toxicology profile and/or good pharmacokinetics may also be effective in the treatment of other indications for example, but not limited to malaria, fungal infection, diseases of the central nervous system and neurodegenerative diseases, diseases dependent on angiogenesis, autoimmune diseases such as rheumatoid arthritis or as a prophylactic pretreatment for cancer.

The utility of ansamycin compounds in the treatment of other conditions is described in the following, including, but not limited to, the treatment of cardiac arrest and stroke (U.S. Pat. No. 6,174,875, WO 99/51223), the treatment of fibrogenic disorders (WO 02/02123), the treatment or prevention of restenosis (WO 03/079936), the treatment or prevention of diseases associated with protein aggregation and amyloid function (WO 02/094259), the treatment of peripheral nerve damage and the promotion of nerve regeneration (WO 01/03692, U.S. Pat. No. 6,641,810, EP 1 024 806, US 2002/0086015, U.S. Pat. No. 6,210,974, WO 99/21552, U.S. Pat. No. 5,968,921) and the inhibition of angiogenesis (WO 04/000307). The uses and methods involving the compounds of the invention also extend to these other indications.

Diseases of the central nervous system and neurodegenerative diseases include, but are not limited to, Alzheimer's disease, Parkinson's disease, Huntington's disease, prion diseases, spinal and bulbar muscular atrophy (SBMA) and amyotrophic lateral sclerosis (ALS). Diseases dependent on angiogenesis include, but are not limited to, age-related macular degeneration, diabetic retinopathy and various other ophthalmic disorders, atherosclerosis and rheumatoid arthritis.

Autoimmune diseases include, but are not limited to, rheumatoid arthritis, multiple sclerosis, type I diabetes, systemic lupus erythematosus and psoriasis.

“Patient” embraces human and other animal (especially mammalian) subjects, preferably human subjects. Accordingly the methods and uses of the C21-deoxy ansamycin analogues of the invention are of use in human and veterinary medicine, preferably human medicine.

In a preferred embodiment, the present invention provides compounds with utility in the treatment of cancer. One skilled in the art would be able by routine experimentation to determine the ability of these compounds to inhibit tumour cell growth, (see Tian et al., 2004; Hu et al. 2004; Dengler et al, 1995).

The present invention also provides a pharmaceutical composition comprising an ansamycin derivative, or a pharmaceutically acceptable salt thereof, together with a pharmaceutically acceptable carrier.

Some existing ansamycin Hsp90 inhibitors that are or have been in clinical trials, such as geldanamycin and 17-AAG have poor pharmacological profiles, poor water solubility and poor bioavailability. The present invention provides novel C21-deoxy ansamycin derivatives which have improved properties such as solubility and/or bioavailability. A person of skill in the art will be able to readily determine the solubility of a given compound of the invention using standard methods. A representative method is shown in the examples herein.

Additionally, a person of skill in the art will be able to determine the pharmacokinetics and bioavailability of a compound of the invention using in vivo and in vitro methods known to a person of skill in the art, including but not limited to those described below and in Egorin M J et al., (2002). The bioavailability of a compound is determined by a number of factors, (e.g. water solubility, rate of absorption in the gut, the extent of protein binding and metabolism) each of which may be determined by in vitro tests as described below, it will be appreciated by a person of skill in the art that an improvement in one or more of these factors will lead to an improvement in the bioavailability of a compound. Alternatively, the bioavailability of a compound may be measured using in vivo methods as described in more detail below.

### In Vitro Assays

### a) Caco-2 Permeation Assay

Confluent Caco-2 cells (Li, A. P., 1992; Grass, G. M., et al., 1992, Volpe, D. A., et al., 2001) in a 24 well Corning Costar Transwell format are used to establish the permeability and efflux rate of compounds using methods as described herein, suitable formats include those provided by In Vitro Technologies Inc. Baltimore, Md., USA. In a suitable format the apical chamber contains 0.15 mL HBBS pH 7.4, 1% DMSO, 0.1 mM Lucifer Yellow and the basal chamber contains 0.6 mL HBBS pH 7.4, 1% DMSO. Controls and test assays are incubated at 37° C. in a humidified incubator, shaken at 130 rpm. Lucifer Yellow is able to permeate via the paracellular route only (i.e. between the tight junctions), a high Apparent Permeability (Pₐₚₚ) for Lucifer Yellow indicates cellular damage during assay and all such wells are rejected. Suitable reference controls in addition to the parent compound include propranolol, which has good passive permeation with no known transporter effects and acebutolol, which has poor passive permeation attenuated by active efflux by P-glycoprotein.

Compounds are tested in a uni- and bi-directional format by applying compound to the apical or basal chamber (at 0.01 mM). Compounds in the apical or basal chambers are analysed by LC-MS. Results are expressed as Apparent Permeability, Pₐₚₚ, (nm/s) and as the Flux Ratio (A to B versus B to A).

$$Papp ( nm \text{/} \text{s} ) = \frac{Volume Acceptor}{Area \times [ donor ]} \times \frac{Δ [ acceptor ]}{Δ time}$$

### Volume Acceptor: 0.6 mL (A>B) and 0.15 mL (B>A)

Area of monolayer: 0.33 cm²
Δtime: 60 min

A positive value for the Flux Ratio indicates active efflux from the apical surface of the cells. Therefore, improved bioavailability is shown in the above assay by an increased Pₐₚₚ and/or a decreased flux ratio for the compound of the invention relative to its parent molecule.

### b) Human Liver Microsomal (HLM) Stability Assay

Increased metabolic stability is also associated with improved bioavailability, this may be determined using a HLM assay for example as described below. Liver homogenates provide a measure of a compounds inherent vulnerability to Phase I (oxidative) enzymes, including CYP450s (e.g. CYP2C8, CYP2D6, CYP1A, CYP3A4, CYP2E1), esterases, amidases and flavin monooxygenases (FMOs).

The half life (T½) of compounds can be determined, on exposure to Human Liver Microsomes, by monitoring their disappearance over time by LC-MS. Compounds at 0.001 mM are incubated at for 40 min at 37° C., 0.1 M Tris-HCl, pH 7.4 with a human microsomal sub-cellular fraction of liver at 0.25 mg/mL protein and saturating levels of NADPH as co-factor. At timed intervals, acetonitrile is added to test samples to precipitate protein and stop metabolism. Samples are centrifuged and analysed for parent compound.

Improved bioavailability is shown in the above assay by an increased T½ relative to the parent compound.

### In Vivo Assays

In vivo assays may also be used to measure the bioavailability of a compound. Generally, a compound is administered to a test animal (e.g. mouse or rat) both intraperitoneally (i.p.) or intravenously (i.v.) and orally (p.o.) and blood samples are taken at regular intervals to examine how the plasma concentration of the drug varies over time. The time course of plasma concentration over time can be used to calculate the absolute bioavailability of the compound as a percentage using standard models. An example of a typical protocol is described below.

Mice are dosed with 1, 10, or 75 mg/kg of the compound of the invention or the parent compound i.p. i.v. or p.o. Blood samples are taken at 5, 10, 15, 30, 45, 60, 90, 120, 180, 240, 360, 420 and 2880 minutes and the concentration of the compound of the invention or parent compound in the sample is determined via HPLC. The time-course of plasma concentrations can then be used to derive key parameters such as the area under the plasma concentration-time curve (AUC—which is directly proportional to the total amount of unchanged drug that reaches the systemic circulation), the maximum (peak) plasma drug concentration, the time at which maximum plasma drug concentration occurs (peak time), additional factors which are used in the accurate determination of bioavailability include: the compound's terminal half life, total body clearance, steady-state volume of distribution and F %. These parameters are then analysed by non-compartmental or compartmental methods to give a calculated percentage bioavailability, for an example of this type of method see Egorin et al., 2002, and references therein.

The aforementioned compounds of the invention or a formulation thereof may be administered by any conventional method for example but without limitation they may be administered parenterally (including intravenous administration), orally, topically (including buccal, sublingual or transdermal), via a medical device (e.g. a stent), by inhalation, or via injection (subcutaneous or intramuscular). The treatment may consist of a single dose or a plurality of doses over a period of time.

Whilst it is possible for a compound of the invention to be administered alone, it is preferable to present it as a pharmaceutical formulation, together with one or more acceptable carriers. Thus there is provided a pharmaceutical composition comprising a compound of the invention together with one or more pharmaceutically acceptable diluents or carriers. The diluents(s) or carrier(s) must be “acceptable” in the sense of being compatible with the compound of the invention and not deleterious to the recipients thereof. Examples of suitable carriers are described in more detail below.

The compounds of the invention may be administered alone or in combination with other therapeutic agents. Co-administration of two (or more) agents may allow for significantly lower doses of each to be used, thereby reducing the side effects seen. Compounds of the invention might also allow resensitisation of a disease, such as cancer, to the effects of a prior therapy to which the disease has become resistant. There is also provided a pharmaceutical composition comprising a compound of the invention and a further therapeutic agent together with one or more pharmaceutically acceptable diluents or carriers.

In a further aspect, the present invention provides for the use of a compound of the invention in combination therapy with a second agent eg a second agent for the treatment of cancer or B-cell malignancies such as a cytotoxic or cytostatic agent.

In one embodiment, a compound of the invention is co-administered with another therapeutic agent eg a therapeutic agent such as a cytotoxic or cytostatic agent for the treatment of cancer or B-cell malignancies. Exemplary further agents include cytotoxic agents such as alkylating agents and mitotic inhibitors (including topoisomerase II inhibitors and tubulin inhibitors). Other exemplary further agents include DNA binders, antimetabolites and cytostatic agents such as protein kinase inhibitors and tyrosine kinase receptor blockers. Suitable agents include, but are not limited to, methotrexate, leucovorin, prenisone, bleomycin, cyclophosphamide, 5-fluorouracil, paclitaxel, docetaxel, vincristine, vinblastine, vinorelbine, doxorubicin (adriamycin), tamoxifen, toremifene, megestrol acetate, anastrozole, goserelin, anti-HER2 monoclonal antibody (e.g. trastuzumab, trade name Herceptin™), capecitabine, raloxifene hydrochloride, EGFR inhibitors (e.g. gefitinib, trade name Iressa®, erlotinib, trade name Tarceva™, cetuximab, trade name Erbitux™), VEGF inhibitors (e.g. bevacizumab, trade name Avastin™) proteasome inhibitors (e.g. bortezomib, trade name Velcade™) or imatinib, trade name Glivec®. Further suitable agents include, but are not limited to, conventional chemotherapeutics such as cisplatin, cytarabine, cyclohexylchloroethylnitrosurea, gemcitabine, Ifosfamid, leucovorin, mitomycin, mitoxantone, oxaliplatin, taxanes including taxol and vindesine; hormonal therapies; monoclonal antibody therapies; protein kinase inhibitors such as dasatinib, lapatinib; histone deacetylase (HDAC) inhibitors such as vorinostat; angiogenesis inhibitors such as sunitinib, sorafenib, lenalidomide; and mTOR inhibitors such as temsirolimus. Additionally, a compound of the invention may be administered in combination with other therapies including, but not limited to, radiotherapy or surgery.

The formulations may conveniently be presented in unit dosage form and may be prepared by any of the methods well known in the art of pharmacy. Such methods include the step of bringing into association the active ingredient (compound of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile. Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

It should be understood that in addition to the ingredients particularly mentioned above the formulations of this invention may include other agents conventional in the art having regard to the type of formulation in question, for example those suitable for oral administration may include flavouring agents.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollient in creams or ointments and ethanol or oleyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base. Alternatively, the active agent may be formulated in a cream with an oil-in-water cream base or a water-in-oil base.

For parenteral administration, fluid unit dosage forms are prepared utilizing the active ingredient and a sterile vehicle, for example but without limitation water, alcohols, polyols, glycerine and vegetable oils, water being preferred. The active ingredient, depending on the vehicle and concentration used, can be either suspended or dissolved in the vehicle. In preparing solutions the active ingredient can be dissolved in water for injection and filter sterilised before filling into a suitable vial or ampoule and sealing.

Advantageously, agents such as local anesthetics, preservatives and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

Parenteral suspensions are prepared in substantially the same manner as solutions, except that the active ingredient is suspended in the vehicle instead of being dissolved and sterilization cannot be accomplished by filtration. The active ingredient can be sterilised by exposure to ethylene oxide before suspending in the sterile vehicle. Advantageously, a surfactant or wetting agent is included in the composition to facilitate uniform distribution of the active ingredient.

The compounds of the invention may also be administered using medical devices known in the art. For example, in one embodiment, a pharmaceutical composition of the invention can be administered with a needleless hypodermic injection device, such as the devices disclosed in U.S. Pat. No. 5,399,163; U.S. Pat. No. 5,383,851; U.S. Pat. No. 5,312,335; U.S. Pat. No. 5,064,413; U.S. Pat. No. 4,941,880; U.S. Pat. No. 4,790,824; or U.S. Pat. No. 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

The dosage to be administered of a compound of the invention will vary according to the particular compound, the disease involved, the subject, and the nature and severity of the disease and the physical condition of the subject, and the selected route of administration. The appropriate dosage can be readily determined by a person skilled in the art.

The compositions may contain from 0.1% by weight, preferably from 5-60%, more preferably from 10-30% by weight, of a compound of invention, depending on the method of administration.

It will be recognized by one of skill in the art that the optimal quantity and spacing of individual dosages of a compound of the invention will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

As also described in our unpublished patent application No. PCT/GB2006/050476, the inventors of the present invention provide methods for the production of C21-deoxy ansamycin templates which can be used as templates to generate the pro-drug derivatives of the invention, specifically described below are methods for generating C21-deoxymacbecin analogues. Similar methods can be employed by one skilled in the art to the generation of other C21-deoxy ansamycin templates.

Macbecin can be considered to be biosynthesised in two stages. In the first stage the core-PKS genes assemble the macrolide core by the repeated assembly of 2-carbon units which are then cyclised to form the first enzyme-free intermediate “pre-macbecin”, see FIG. 1. In the second stage a series of “post-PKS” tailoring enzymes (e.g. P450 monooxygenases, methyltransferases, FAD-dependent oxygenases and a carbamoyltransferase) act to add the various additional groups to the pre-macbecin template resulting in the final parent compound structure, see FIG. 2. The C21-deoxymacbecin analogues to be used as templates may be biosynthesised in a similar manner.

This biosynthetic production may be exploited by genetic engineering of suitable producer strains to result in the production of novel compounds. In particular, the present invention provides a method of producing C21-deoxymacbecin analogues said method comprising:

a) providing a first host strain that produces macbecin or an analogue thereof when cultured under appropriate conditions
b) deleting or inactivating one or more post-PKS genes, wherein at least one of those post-PKS genes is mbcM, or a homologue thereof
c) culturing said modified host strain under suitable conditions for the production of C21-deoxymacbecin analogues; and
d) optionally isolating the compounds produced.

In step (b), deleting or inactivating one or more post-PKS genes, wherein at least one of those post-PKS genes is mbcM, or a homologue thereof will suitably be done selectively.

Step b) may comprise inactivating mbcM (or a homologue thereof) by integration of DNA into the mbcM gene (or a homologue thereof) such that functional MbcM protein is not produced. Alternatively, step b) comprises making a targeted deletion of the mbcM gene, or a homologue thereof. Or, mbcM, or a homologue thereof, is inactivated by site-directed mutagenesis. Alternatively, the host strain of step a) is subjected to mutagenesis and a modified strain is selected in which one or more of the post-PKS enzymes is not functional, wherein at least one of these is MbcM. The present invention also encompasses mutations of the regulators controlling the expression of mbcM, or a homologue thereof, a person of skill in the art will appreciate that deletion or inactivation of a regulator may have the same outcome as deletion or inactivation of the gene.

For example, a method of selectively deleting or inactivating a post PKS gene comprises:

(i) designing degenerate oligos based on homologue(s) of the gene of interest (e.g. from the geldanamycin PKS biosynthetic cluster and/or from the rifamycin biosynthetic cluster) and isolating the internal fragment of the gene of interest (e.g. mbcM) from a suitable macbecin producing strain, by using these primers in a PCR reaction;
(ii) integrating a plasmid containing this fragment into either the same, or a different macbecin producing strain followed by homologous recombination, which results in the disruption of the targeted gene (e.g. mbcM or a homologue thereof),
(iii) culturing the strain thus produced under conditions suitable for the production of the macbecin analogues, i.e. C21-deoxymacbecin analogues.

The macbecin-producing strain in step (i) may be Actinosynnema mirum (A. mirum) and the macbecin-producing strain in step (ii) may be A. pretiosum

A person of skill in the art will appreciate that an equivalent strain may be achieved using alternative methods to that described above, e.g.:
Degenerate oligos may be used to amplify the gene of interest from other macbecin producing strains for example, but not limited to A. pretiosum, or A. mirum
Different degenerate oligos may be designed which will successfully amplify an appropriate region of the mcbM gene of a macbecin producer, or a homologue thereof.
The sequence of the mbcM gene of the A. pretiosum strain may be used to generate the oligos which may be specific to the mbcM gene of A. pretiosum and then the internal fragment may be amplified from any macbecin producing strain e.g A. pretiosum or Actinosynnema mirum (A. mirum).
The sequence of the mbcM gene of the A. pretiosum strain may be used along with the sequence of homologous genes to generate degenerate oligos to the mbcM gene of A. pretiosum and then the internal fragment may be amplified from any macbecin producing strain e.g A. pretiosum or A. mirum.

Further post-PKS genes may also be deleted or inactivated in addition to mbcM. FIG. 2 shows the activity of the post-PKS genes in the macbecin biosynthetic cluster. A person of skill in the art would thus be able to identify which additional post-PKS genes would need to be deleted or inactivated in order to arrive at a strain that will produce the compound(s) of interest.

Further C21-deoxymacbecin analogues may be produced using an engineered strain in which one or more post-PKS genes including mbcM have been deleted or inactivated as above, has re-introduced into it one or more of the same post PKS genes not including mbcM, or homologues thereof, e.g. from an alternative macbecin producing strain, or even from the same strain.

For example a method for the production of a C21-deoxymacbecin analogue, may comprise:

a) providing a first host strain that produces macbecin when cultured under appropriate conditions
b) deleting or inactivating one or more post-PKS genes, wherein at least one of the post-PKS genes is mbcM, or a homologue thereof,
c) re-introducing some or all of the post-PKS genes not including mbcM.
d) culturing said modified host strain under suitable conditions for the production of C21-deoxymacbecin analogues; and
e) optionally isolating the compounds produced.

Further, an engineered strain in which one or more post-PKS genes including mbcM have been deleted or inactivated is complemented by one or more of the post PKS genes from a heterologous PKS cluster including, but not limited to the clusters directing the biosynthesis of rifamycin, ansamitocin, geldanamycin or herbimycin.

The host strain may be an engineered strain based on a macbecin producing strain in which mbcM has been deleted or inactivated. Alternatively the host strain may be an engineered strain based on a macbecin producing strain in which mbcM, mbcMT1, mbcMT2, mbcP and mbcP450 have been deleted or inactivated.

It may be observed in these systems that when a strain is generated in which mbcM, or a homologue thereof, does not function as a result of one of the methods described including inactivation or deletion, that more than one macbecin analogue may be produced. There are a number of possible reasons for this which will be appreciated by those skilled in the art. For example there may be a preferred order of post-PKS steps and removing a single activity leads to all subsequent steps being carried out on substrates that are not natural to the enzymes involved. This can lead to intermediates building up in the culture broth due to a lowered efficiency towards the novel substrates presented to the post-PKS enzymes, or to shunt products which are no longer substrates for the remaining enzymes possibly because the order of steps has been altered.

The ratio of compounds observed in a mixture can be manipulated by using variations in the growth conditions such as the setting of revolutions per minute (rpm) in the shaking incubator, and the throw of the shaking incubator. As described in the examples, incubation of production cultures of BIOT-3806 lead to a mixture of compounds, the ratio of these compounds could be altered by changing the growth conditions.

One skilled in the art will appreciate that in a biosynthetic cluster some genes are organised in operons and disruption of one gene will often have an effect on expression of subsequent genes in the same operon.

When a mixture of compounds is observed these can be readily separated using standard techniques some of which are described in the following examples.

In the circumstance where a single compound is referred a strain can be engineered to make this compound preferably. In the unusual circumstance when this is not possible, an intermediate can be generated which is then biotransformed to produce the desired compound.

The description herein relates to generation C21-deoxymacbecin analogues that can be used as templates for semi-synthesis to generate C21-deoxymacbecin derivatives that are pro-drugs. Templates of particular interest are produced by the selected deletion or inactivation of at least mbcM, or a homologue thereof, from the macbecin biosynthetic gene cluster. For example, mbcM, or a homologue thereof, alone is deleted or inactivated. Alternatively, other post-PKS genes in addition to mbcM are additionally deleted or inactivated. Specifically, additional genes selected from the group consisting of: mbcN, mbcP, mbcMT1, mbcMT2 and mbcP450 are deleted or inactivated in the host strain.

A person of skill in the art will appreciate that a gene does not need to be completely deleted for it to be rendered non-functional, consequentially the term “deleted or inactivated” as used herein encompasses any method by which a gene is rendered non-functional including but not limited to: deletion of the gene in its entirety, inactivation by insertion into the target gene, site-directed mutagenesis which results in the gene either not being expressed or being expressed in an inactive form, mutagenesis of the host strain which results in the gene either not being expressed or being expressed in an inactive form (e.g. by radiation or exposure to mutagenic chemicals, protoplast fusion or transposon mutagenesis). Further it includes deletion of an internal fragment of the gene. Alternatively the function of an active gene can be impaired chemically with inhibitors, for example metapyrone (alternative name 2-methyl-1,2-di(3-pyridyl-1-propanone), EP 0 627 009) and ancymidol are inhibitors of oxygenases and these compounds can be added to the production medium to generate analogues. Additionally, sinefungin is a methyl transferase inhibitor that can be used similarly but for the inhibition of methyl transferase activity in vivo (McCammon and Parks 1981).

All of the post-PKS genes may be deleted or inactivated and then one or more of the genes, but not including mbcM, or a homologue thereof, may then be reintroduced by complementation (e.g. at an att site, on a self-replicating plasmid or by insertion into a homologous region of the chromosome). Methods for the generation of C21-deoxymacbecin analogues for use as templates in semi-synthesis to generate pro-drug derivatives, may comprise:

a) providing a first host strain that produces macbecin when cultured under appropriate conditions
b) selectively deleting or inactivating all the post-PKS genes,
c) culturing said modified host strain under suitable conditions for the production of C21-deoxymacbecin analogues; and
d) optionally isolating the compounds produced.

Further, one or more of the deleted post-PKS genes may be reintroduced, provided that mbcM is not one of the genes reintroduced, for example one or more of mbcN, mbcP, mbcMT1, mbcMT2 and mbcP450 are reintroduced.

Additionally, it will be apparent to a person of skill in the art that a subset of the post-PKS genes, including mbcM, or a homologue thereof, could be deleted or inactivated and a smaller subset of said post-PKS genes not including mbcM could be reintroduced to arrive at a strain producing C21-deoxymacbecin analogues.

A person of skill in the art will appreciate that there are a number of ways to generate a strain that contains the biosynthetic gene cluster for macbecin but that is lacking at least mbcM, or a homologue thereof.

It is well known to those skilled in the art that polyketide gene clusters may be expressed in heterologous hosts (Pfeifer and Khosla, 2001). Accordingly, the present invention includes the transfer of the macbecin biosynthetic gene cluster without mbcM or with a non-functional mutant of mbcM, with or without resistance and regulatory genes, either otherwise complete or containing additional deletions, into a heterologous host. Alternatively, the complete macbecin biosynthetic cluster can be transferred into a heterologous host, with or without resistance and regulatory genes, and it can then be manipulated by the methods described herein to delete or inactivate mbcM. Methods and vectors for the transfer as defined above of such large pieces of DNA are well known in the art (Rawlings, 2001; Staunton and Weissman, 2001) or are provided herein in the methods disclosed. In this context a preferred host cell strain is a prokaryote, more preferably an actinomycete or Escherichia coli, still more preferably preferred host cell strains include, but are not limited to Actinosynnema mirum (A. mirum), Actinosynnema pretiosum subsp. pretiosum (A. pretiosum), S. hygroscopicus, S. hygroscopicus sp., S. hygroscopicus var. ascomyceticus, Streptomyces tsukubaensis, Streptomyces coelicolor, Streptomyces lividans, Saccharopolyspora erythraea, Streptomyces fradiae, Streptomyces avermitilis, Streptomyces cinnamonensis, Streptomyces rimosus, Streptomyces albus, Streptomyces griseofuscus, Streptomyces longisporoflavus, Streptomyces venezuelae, Streptomyces albus, Micromonospora sp., Micromonospora griseorubida, Amycolatopsis mediterranei or Actinoplanes sp. N902-109. Further examples include Streptomyces hygroscopicus subsp. geldanus and Streptomyces violaceusniger.

For example the entire biosynthetic cluster may be transferred. Alternatively, the entire PKS without mbcM is transferred. Or, the entire PKS is transferred without any of the associated post-PKS genes, including mbcM.

Or the entire macbecin biosynthetic cluster is transferred and then manipulated according to the description herein.

The C21-deoxymacbecin analogue may be further processed by biotransformation with an appropriate strain. The appropriate strain either being an available wild type strain for example, but without limitation Actinosynnema mirum, Actinosynnema pretiosum subsp. pretiosum, S. hygroscopicus, S. hygroscopicus sp. Alternatively, an appropriate strain may be a engineered to allow biotransformation with particular post-PKS enzymes for example, but without limitation, those encoded by mbcN, mbcP, mbcMT1, mbcMT2, mbcP450 (as defined herein), gdmN, gdmM, gdmL, gdmP, (Rascher et al., 2003) the geldanamycin 17-O-methyl transferase, asm7, asm10, asm11, asm12, asm19 and asm21 (Cassady et al., 2004, Spiteller et al., 2003). Where genes have yet to be identified or the sequences are not in the public domain it is routine to those skilled in the art to acquire such sequences by standard methods. For example the sequence of the gene encoding the geldanamycin 17-O-methyl transferase is not in the public domain, but one skilled in the art could generate a probe, either a heterologous probe using a similar O-methyl transferase, or a homologous probe by designing degenerate primers from available homologous genes to carry out Southern blots on a geldanamycin producing strain and thus acquire this gene to generate biotransformation systems.

The strain used as a host, or for biotransformation may have had one or more of its native polyketide clusters deleted, either entirely or in part, or otherwise inactivated, so as to prevent the production of the polyketide produced by said native polyketide cluster. Said engineered strain may be selected from the group including, for example but without limitation, Actinosynnema mirum, Actinosynnema pretiosum subsp. pretiosum, S. hygroscopicus, S. hygroscopicus sp., S. hygroscopicus var. ascomyceticus, Streptomyces tsukubaensis, Streptomyces coelicolor, Streptomyces lividans, Saccharopolyspora erythraea, Streptomyces fradiae, Streptomyces avermitilis, Streptomyces cinnamonensis, Streptomyces rimosus, Streptomyces albus, Streptomyces griseofuscus, Streptomyces longisporoflavus, Streptomyces venezuelae, Micromonospora sp., Micromonospora griseorubida, Amycolatopsis mediterranei, Actinoplanes sp. N902-109, Streptomyces hygroscopicus subsp. geldanus or Streptomyces violaceusniger.

Although the process for preparation of the C21-deoxymacbecin templates as described above is substantially or entirely biosynthetic, it is not ruled out to produce or interconvert C21-deoxymacbecin analogues of the invention by a process which comprises standard synthetic chemical methods.

In order to allow for the genetic manipulation of the macbecin biosynthetic gene cluster, first the gene cluster was sequenced from Actinosynnema pretiosum subsp. pretiosum however, a person of skill in the art will appreciate that there are alternative strains which produce macbecin, for example but without limitation Actinosynnema mirum. The macbecin biosynthetic gene cluster from these strains may be sequenced as described herein for Actinosynnema pretiosum subsp. pretiosum, and the information used to generate equivalent strains.

The methods described in the above description of manipulation of the macbecin pathway in order to generate C21-deoxymacbecin analogues as templates for semi-synthesis can be applied by one skilled in the art to any ansamycin polyketide cluster in order to generate C21-deoxy ansamycin analogues.

Compounds of the invention are advantageous in that they may be expected to have one or more of the following properties: tight binding to Hsp90, fast on-rate of binding to Hsp90, good water solubility, good stability, good formulation ability, good oral bioavailability, good pharmacokinetic properties including but not limited to low glucuronidation, good cell up-take, good brain pharmacokinetics, low binding to erythrocytes, good toxicology profile, good hepatotoxicity profile, good nephrotoxicity, low side effects and low cardiac side effects.

### EXAMPLES

### General Methods

### Fermentation of Cultures

Conditions used for growing the bacterial strains Actinosynnema pretiosum subsp. pretiosum ATCC 31280 (U.S. Pat. No. 4,315,989) and Actinosynnema mirum DSM 43827 (KCC A-0225, Watanabe et al., 1982) were described in the patents U.S. Pat. No. 4,315,989 and U.S. Pat. No. 4,187,292. Methods used herein were adapted from these patents and are as follows for culturing of broths in tubes or flasks in shaking incubators, variations to the published protocols are indicated in the examples. Both strains were grown on ISP2 agar (Medium 3, Shirling, E. B. and Gottlieb, D., 1966) at 28° C. for 2-3 days and used to inoculate seed medium (Medium 1, see below adapted from U.S. Pat. No. 4,315,989 and U.S. Pat. No. 4,187,292). The inoculated seed medium was then incubated with shaking between 200 and 300 rpm with a 5 or 2.5 cm throw at 28° C. for 48 h. For production of C21-deoxymacbecin analogues the fermentation medium (Medium 2, see below and U.S. Pat. No. 4,315,989 and U.S. Pat. No. 4,187,292) was inoculated with 2.5%-10% of the seed culture and incubated with shaking between 200 and 300 rpm with a 5 or 2.5 cm throw initially at 28° C. for 24 h followed by 26° C. for four to six days. The culture was then harvested for extraction.

### Media

Medium 1 - Seed Medium
In 1 L of distilled water

| | | |
|---|---|---|
| Glucose | 20 | g |
| Soluble potato starch (Sigma) | 30 | g |
| Spray dried corn steep liquor (Roquette Freres) | 10 | g |
| ‘Nutrisoy’ toasted soy flour (Archer Daniels | 10 | g |
| Midland) | | |
| Peptone from milk solids (Sigma) | 5 | g |
| NaCl | 3 | g |
| CaCO₃ | 5 | g |

| | |
|---|---|
| Adjust pH with NaOH | 7.0 |

Sterilisation by autoclaving at 121° C. for 20 minutes.

Apramycin was added when appropriate after autoclaving to give a final concentration of 50 mg/L.

Medium 2 - Fermentation Medium
In 1 L of distilled water

| Glycerol | 50 g |
|---|---|
| Spray dried corn steep liquor (Roquette Freres) | 10 g |
| ‘Bacto’ yeast extract (Difco) | 20 g |
| KH₂PO₄ | 20 g |
| MgCl₂•6H₂O | 5 g |
| CaCO₃ | 1 g |
| Adjust pH with NaOH | 6.5 |

Sterilisation by autoclaving at 121° C. for 20 minutes.

Medium 3 - ISP2 Medium
In 1 L of distilled water

| | | |
|---|---|---|
| Malt extract | 10 | g |
| Yeast extract | 4 | g |
| Dextrose | 4 | g |
| Agar | 15 | g |

| | |
|---|---|
| Adjust pH with NaOH | 7.3 |

Sterilisation by autoclaving at 121° C. for 20 minutes.

Medium 4 - MAM
In 1 L of distilled water

| | | |
|---|---|---|
| Wheat starch | 10 | g |
| Corn steep solids | 2.5 | g |
| Yeast extract | 3 | g |
| CaCO₃ | 3 | g |
| Iron sulphate | 0.3 | g |
| Agar | 20 | g |

Sterilisation by autoclaving at 121° C. for 20 minutes.

### Extraction of Culture Broths for LCMS Analysis

Culture broth (1 mL) and ethyl acetate (1 mL) was added and mixed for 15-30 min followed by centrifugation for 10 min. 0.5 mL of the organic layer was collected, evaporated to dryness and then re-dissolved in 0.25 mL of methanol.

### LCMS Analysis Procedure for Fermentation Broth Analysis and In Vivo Transformation Studies

HPLC was performed on a Phenomenex Hyperclone 3 micron BDS C18 column, 150 mm×4.60 mm, running a mobile phase of:

Mobile phase A: 0.1% Formic acid in water
Mobile phase B: 0.1% Formic acid in acetonitrile
Flow rate: 1 mL/minute.

The HPLC conditions were: 10% B for 1 min followed by a linear gradient to 100% B over a period of 7 min and an isocratic period of 2 min at 100% B. The analytes were detected by UV absorbance at 255 nm and mass spectrometry using a Bruker Daltonics Esquire 3000+ mass spectrometer coupled to the HPLC.

### Synthesis

Unless stated otherwise, all reactions were conducted under anhydrous conditions, in oven dried glassware that is cooled under vacuum, using dried solvents. Reactions were monitored by LC-UV-MS, on an Agilent 1100 HPLC coupled to a Bruker Daltonics Esquire3000 electrospray mass spectrometer, switching between positive ion and negative ion modes for alternate scans. Chromatography was achieved over a Phenomenex Hyperclone column, BDS C₁₈ 3u (150×4.6 mm), with a linear gradient of mobile phase A/mobile phase B (40:60 to 100) over 11 min at 1 mL/min.

Mobile phase A: 0.1% Formic acid in water
Mobile phase B: 0.1% Formic acid in acetonitrile
Flow rate: 1 mL/minute.

### Water Solubility Assays

Kinetic Measurements:

Stock solutions of the compounds (10 mM) in DMSO were prepared. Aliquots (0.01 mL) of each were made up to 0.5 mL with either PBS solution or DMSO. The resulting 0.2 mM solutions were shaken for at room temperature on an IKA® vibrax VXR shaker.
After shaking the resulting solutions or suspensions were transferred to 2 mL Eppendorf tubes and centrifuged for 30 minutes at 13200 rpm. Aliquots of the supernatant fluid were then analysed by an Agilent 1100 HPLC coupled to a Bruker Daltonics Esquire3000 electrospray mass spectrometer (for details see specific examples herein). Chromatography was achieved over a Phenomenex Hyperclone column, BDS C₁₈ 3u (150×4.6 mm), with a linear gradient of acetonitrile:water (40:60 to 100) over 11 min at 1 mL/min. UV absorbance was monitored at λ=258 and 280 nm.
All analyses were performed in triplicate and the solubilities of individual compounds calculated by comparing their solubility in PBS with an assumed solubility of 100% in DMSO at 0.2 mM.

Thermodynamic Measurements:
The appropriate amounts of compound to make solutions of the compounds at 2, 5, 10 and or 20 mM were mixed with the appropriate amounts of 5% glucose and with DMSO in brown glass vials and shaken at room temperature on an IKA® vibrax VXR shaker. After 6 hours the resulting suspensions/solutions were centrifuged for 20 min at 13200 rpm.
Aliquots of the supernatant fluid were then analysed by an Agilent 1100 HPLC coupled to a Bruker Daltonics Esquire3000 electrospray mass spectrometer (for details see specific examples herein). Chromatography was achieved over a Phenomenex Hyperclone column, BDS C₁₈ 3u (150×4.6 mm), with a linear gradient of acetonitrile:water (40:60 to 100) over 11 min at 1 mL/min. UV absorbance was monitored at λ=258 and 280 nm.
All analyses were performed in triplicate and the solubilities of individual compounds calculated by comparing their solubility in 5% glucose with an assumed solubility of 100% in DMSO.

### Whole Blood Cleavage Assay

Human whole blood (single donor, batch HBE4534) was obtained from First Link UK Ltd. Mouse whole blood (pool of 10, batch 07-2470) was obtained from Harlan UK. Blood was collected into tubes containing EDTA as anticoagulant and shipped on ice. Blood was used on the day of arrival. In the case of human whole blood incubations were undertaken both on the fresh blood and also after freezing the blood followed by thawing. Control compounds were Lidocaine, Bisacodyl and Simvastatin.

All test compounds were incubated at 10 uM in whole blood at 37° C. At selected time points, 0.1 ml of blood was mixed with 0.3 ml of acetonitrile and vortexed immediately. All samples were centrifuged and supernatant were diluted with same volume of deionized water. Sample analysis was carried out on LC/MS/MS. For 20 sample analysis, mass spectrometry monitored both 17 and 20. A blank whole blood extract and a reference sample (containing 1.25 uM of 20 and 17) were also prepared and injected with incubation samples.

The Half life, for the disappearance of 20, was determined according to the relationship:

Half life(min)=0.693/λ(λ is the slope of the ln concn vs time curve)

### Compound Detection and Experimental Methodology

A Micromass Quattro Micro mass spectrometer (Waters Ltd) was used. The settings of the negative mode electrospray ion source (ESI) used for method development and subsequent data acquisition are detailed below. A Waters 2795 HPLC system was used as front end for mass spectrometry.

### In Vitro Caco-2 Assay for Cell Permeability

Caco-2 cells (ATCC Cat. # HTB-37) were grown on fibrillar collagen-coated, microporous, polycarbonate membranes in 24-well BioCaot™ insert plates. Following 5-day growth in Eagle's minimum essential medium supplemented with 10% FBS, the cells were exposed to BioCoat Enterocyte Differentiation Medium (BD, Cat. # 05496) for 2 days to induce enterocytic differentiation. Prior to dosing, the transepithelial electric resistance (TEER) of the Caco-2 cells in each well was measured to ensure the quality of the monolayers. Only qualified wells that had a TEER greater than 1400Ω were used.

The stock solutions of test compounds were prepared at 3 mM in DMSO. Dosing solutions were prepared in the permeability assay buffer at 10 μm. The permeability assay buffer was Hank's balanced salt solution containing 10 mM HEPES at a pH of 7.4±0.2.

The Caco-2 cells were dosed with the test compounds on either apical side (for A-to-B permeation) or basolateral side (for B-to-A permeation) and incubated at 37° C. with 5% CO₂ and 90% relative humidity. The testing for each compound was performed in duplicate. After 2 hour incubation, a 20-μL sample was taken from each of dosing solutions and both donor and receiver compartments. To ensure the validity of the Caco-2 assay, propranolol and vinblastine were used as a high- and a low- to medium-permeability positive control, respectively. Vinblastine also served as a P-gp substrate, tested in conjunction with a P-gp inhibitor, verapamil.

To quantify test compounds by LC/MS/MS, a related geldanamycin-like compound, 17-methoxyethylamino geldanamycin (Schnur et al., 1995), was used as the internal standard. The calculation of permeability of each compound involved only the concentration ratio of the same test compound, the concentration ratio was expressed in the peak area ratio of the test compound to the internal standard. The peak area ratio of each compound was derived by LC-MS/MS.

### Calculation of Permeability

### Calculation of Pₐₚₚ

The apparent permeability coefficient Pₐₚₚ was calculated as below:

$${P}_{app} = \frac{( d {C}_{r} / d t ) \times {V}_{r}}{A \times {C}_{0}}$$

### Where,

dCᵣ: cumulative concentration in the receiver compartment in M.
dt: duration of the assay (i.e., 7200 seconds).
Vᵣ: volume of the receiver compartment in cm³.
A: area of the cell monolayer (0.31 cm² for 24-well BioCoat™ plate).
C₀: concentration of the dosing solution in M.

### Calculation of Pₑ

The permeability coefficient Pₑ was calculated as follows:

$${P}_{e} = \frac{{V}_{d} \times {V}_{r}}{( {V}_{d} + {V}_{r} ) \times A \times T} \times [ - Ln ( 1 - \frac{{C}_{r}}{{C}_{e}} ) ]$$

### Where,

V_{d}: volume of the donor compartment in cm³ (i.e., 0.15 cm³)
Vᵣ: volume of the receiver compartment in cm³ (i.e., 0.3 cm³)
Cᵣ: concentration (M) of a test compound in receiver compartment at the end of the incubation
Cₑ: averaged concentration (M) of a test compound in both donor and receiver compartments at the end of the incubation
A: area of membrane (i.e., 0.30 cm²)
T: duration of the incubation in seconds (i.e., 64800 seconds)

### In Vitro Bioassay for Anticancer Activity

In vitro evaluation of compounds for anticancer activity in a panel of human tumour cell lines in a monolayer proliferation assay was carried out at the Oncotest Testing Facility, Institute for Experimental Oncology, Oncotest GmbH, Freiburg. The characteristics of the selected cell lines are summarized in Table 5.

TABLE 5
Test cell lines

| # | Cell line | Characteristics |
|---|---|---|
| 1 | CNXF 498NL | CNS |
| 2 | CXF HT29 | Colon |
| 3 | LXF 1121L | Lung, large cell carcinoma |
| 4 | MCF-7 | Breast, NCI standard |
| 5 | MEXF 394NL | Melanoma |
| 6 | DU145 | Prostate - PTEN positive |

The Oncotest cell lines are established from human tumor xenografts as described by Roth et al., (1999). The origin of the donor xenografts is described by Fiebig et al., (1999). Other cell lines are either obtained from the NCl (DU145, MCF-7) or purchased from DSMZ, Braunschweig, Germany.

All cell lines, unless otherwise specified, are grown at 37° C. in a humidified atmosphere (95% air, 5% CO₂) in a ‘ready-mix’ medium containing RPMI 1640 medium, 10% fetal calf serum, and 0.1 mg/mL gentamicin (PAA, Cölbe, Germany).

A modified propidium iodide assay was used to assess the effects of the test compound(s) on the growth of six human tumour cell lines (Dengler et al., (1995)).

Briefly, cells are harvested from exponential phase cultures by trypsinization, counted and plated in 96 well flat-bottomed microtitre plates at a cell density dependent on the cell line (5-10.000 viable cells/well). After 24 h recovery to allow the cells to resume exponential growth, 0.010 mL of culture medium (6 control wells per plate) or culture medium containing macbecin were added to the wells. Each concentration is plated in triplicate. Compounds were applied in two concentrations (0.001 mM and 0.01 mM). Following 4 days of continuous exposure, cell culture medium with or without test compound was replaced by 0.2 mL of an aqueous propidium iodide (P1) solution (7 mg/L). To measure the proportion of living cells, cells were permeabilized by freezing the plates. After thawing the plates, fluorescence was measured using the Cytofluor 4000 microplate reader (excitation 530 nm, emission 620 nm), giving a direct relationship to the total number of viable cells.

Growth inhibition is expressed as treated/control×100 (% T/C).

### Pharmacokinetic Analysis

The test compound was prepared directly in endotoxin free water. A single dose of 10 mg/kg p.o. or 3 mg/kg i.v. was administered to groups of female CD1 mice (3 mice for each compound per time point). Dose volumes were 10 mL/kg for both oral and intravenous administration. At 4 minutes and 0.25, 0.5, 1, 2, 4, 8, 24 hours groups were sacrificed and plasma was collected from each mouse for further analysis. For oral dosing, a single dose of Test Article was administered via oral gavage to the mice. For intravenous dosing, a single dose of Test article was administered intravenously to mice via the tail veins.

### Analysis of the Pharmacokinetic Study Samples

The concentration of the relevant compounds in the plasma samples was determined by HPLC with MS detection.

### Bioanalytical Method and Sample Analysis

Mass spectrometer: API 3200 Q Trap triple quadruple mass spectrometer
Liquid chromatography: Agilent 1200/PAL HTC Autosampler
Ionization Mode: Electrospray (may be changed depending on the TA)
Monitoring: Multiple Reaction Monitoring
LC chromatography BetaBastic-8, 50×2.1 mm, 5 μm (may separation: be changed depending on the TA)

Based on the peak area ratios of test article to an internal standard, 17-Methoxyethylamino Geldanamycin (Schnur et al., 1995), concentrations of an unknown sample were obtained by a calibration curve.

### Extraction Method

Test plasma sample (0.05 ml), analyte free mouse whole serum (0.05 ml), internal standard solution (0.1 ml of 10 mg/mL 17-Methoxyethylamino geldanamycin (Schnur et al., 1995) dissolved in methanol) and acetonitrile (0.5 ml) were pipetted into a 2-mL polypropylene tube and the contents of the tubes were mixed for a minimum of 5 minutes (Vibrax mixer). The tubes were then centrifuged in a microfuge for a minimum of 2 minutes at 12,000 rpm. 0.1 ml of the solvent layer was transferred into a 2-mL polypropylene tube containing 1 mL acid diluent (0.1% formic acid). The tubes were then mixed for a minimum of 5 minutes (Vibrax mixer) and then centrifuged at approximately 3500 rpm for 5 minutes. The extracts were transferred to auto-sampler vials and placed in the auto sampler tray which was set at ambient temperature. The auto-sampler was programmed to inject a 0.005 ml aliquot of each extract onto the analytical column.

### Example 1

### Sequencing of the Macbecin Biosynthetic Gene Cluster

Genomic DNA was isolated from Actinosynnema pretiosum (ATCC 31280) and Actinosynnema mirum (DSM 43827, ATCC 29888) using standard protocols described in Kieser et al., (2000) DNA sequencing was carried out by the sequencing facility of the Biochemistry Department, University of Cambridge, Tennis Court Road, Cambridge CB2 1QW using standard procedures.

Primers BIOSG104 5′-GGTCTAGAGGTCAGTGCCCCCGCGTACCGTCGT-3′ (SEQ ID NO: 1) AND BIOSG105 5′-GGCATATGCTTGTGCTCGGGCTCAAC-3′ (SEQ ID NO: 2) were employed to amplify the carbamoyltransferase-encoding gene gdmN from the geldanamycin biosynthetic gene cluster of Streptomyces hygroscopicus NRRL 3602 (Accession number of sequence: AY179507) using standard techniques. Southern blot experiments were carried out using the DIG Reagents and Kits for Non-Radioactive Nucleic Acid Labelling and Detection according to the manufacturers' instructions (Roche). The DIG-labeled gdmN DNA fragment was used as a heterologous probe. Using the gdmN generated probe and genomic DNA isolated from A. pretiosum 2112 an approximately 8 kb EcoRI fragment was identified in Southern Blot analysis. The fragment was cloned into Litmus 28 applying standard procedures and transformants were identified by colony hybridization. The clone p3 was isolated and the approximately 7.7 kb insert was sequenced. DNA isolated from clone p3 was digested with EcoRI and EcoRI/SacI and the bands at around 7.7 kb and at about 1.2 kb were isolated, respectively. Labelling reactions were carried out according to the manufacturers' protocols. Cosmid libraries of the two strains named above were created using the vector SuperCos 1 and the Gigapack III XL packaging kit (Stratagene) according to the manufacturers' instructions. These two libraries were screened using standard protocols and as a probe, the DIG-labelled fragments of the 7.7 kb EcoRI fragment derived from clone p3 were used. Cosmid 52 was identified from the cosmid library of A. pretiosum and submitted for sequencing to the sequencing facility of the Biochemistry Department of the University of Cambridge. Similarly, cosmid 43 and cosmid 46 were identified from the cosmid library of A. mirum. All three cosmids contain the 7.7 kb EcoRI fragment as shown by Southern Blot analysis. An around 0.7 kbp fragment of the PKS region of cosmid 43 was amplified using primers BIOSG124 5′-CCCGCCCGCGCGAGCGGCGCGTGGCCGCCCGAGGGC-3′ (SEQ ID NO: 3) and BIOSG125 5′-GCGTCCTCGCGCAGCCACGCCACCAGCAGCTCCAGC-3′ (SEQ ID NO:4) applying standard protocols, cloned and used as a probe for screening the A. pretiosum cosmid library for overlapping clones. The sequence information of cosmid 52 was also used to create probes derived from DNA fragments amplified by primers BIOSG130 5′-CCAACCCCGCCGCGTCCCCGGCCGCGCCGAACACG-3′ (SEQ ID NO: 5) and BIOSG131 5′-GTCGTCGGCTACGGGCCGGTGGGGCAGCTGCTGT-5′ (SEQ ID NO: 6) as well as BIOSG132 5′-GTCGGTGGACTGCCCTGCGCCTGATCGCCCTGCGC-3′ (SEQ ID NO: 7) and BIOSG133 5′-GGCCGGTGGTGCTGCCCGAGGACGGGGAGCTGCGG-3′ (SEQ ID NO: 8) which were used for screening the cosmid library of A. pretiosum. Cosmids 311 and 352 were isolated and cosmid 352 was sent for sequencing. Cosmid 352 contains an overlap of approximately 2.7 kb with cosmid 52. To screen for further cosmids, an approximately 0.6 kb PCR fragment was amplified using primers BIOSG136 5′-CACCGCTCGCGGGGGTGGCGCGGCGCACGACGTGG CTGC-3′ (SEQ ID NO: 9) and BIOSG 137 5′-CCTCCTCGGACAGCGCGATCAGCGCCGCGC ACAGCGAG-3′ (SEQ ID NO: 10) and cosmid 311 as template applying standard protocols. The cosmid library of A. pretiosum was screened and cosmid 410 was isolated. It overlaps approximately 17 kb with cosmid 352 and was sent for sequencing. The sequence of the three overlapping cosmids (cosmid 52, cosmid 352 and cosmid 410) was assembled. The sequenced region spans about 100 kbp and 23 open reading frames were identified potentially constituting the macbecin biosynthetic gene cluster, (SEQ ID NO: 11). The location of each of the open reading frames within SEQ ID NO: 11 is shown in Table 7

TABLE 6
Summary of the cosmids

| Cosmid | Strain |
|---|---|
| Cosmid 43 | Actinosynnema mirum ATCC 29888 |
| Cosmid 46 | Actinosynnema mirum ATCC 29888 |
| Cosmid 52 | Actinosynnema pretiosum ATCC 31280 |
| Cosmid 311 | Actinosynnema pretiosum ATCC 31280 |
| Cosmid 352 | Actinosynnema pretiosum ATCC 31280 |
| Cosmid 410 | Actinosynnema pretiosum ATCC 31280 |

TABLE 7
location of each of the open reading frames within SEQ ID NO: 11

| Nucleotide position in | | Function of the encoded |
|---|---|---|
| SEQ ID NO: 11 | Gene Name | protein |
| 14925-17909* | mbcRII | transcriptional regulator |
| 18025-19074c | mbcO | aminohydroquinate synthase |
| 19263-20066c* | mbc? | unknown, AHBA biosynthesis |
| 20330-40657 | mbcAI | PKS |
| 40654-50859 | mbcAII | PKS |
| 50867-62491* | mbcAIII | PKS |
| 62500-63276* | mbcF | amide synthase |
| 63281-64852* | mbcM | C21 monooxygenase |
| 64899-65696c* | PH | phosphatase |
| 65693-66853c* | OX | oxidoreductase |
| 66891-68057c* | Ahs | AHBA synthase |
| 68301-68732* | Adh | ADHQ dehydratase |
| 68690-69661c* | AHk | AHBA kinase |
| 70185-72194c* | mbcN | carbamoyltransferase |
| 72248-73339c | mbcH | methoxymalonyl ACP pathway |
| 73336-74493c | mbcI | methoxymalonyl ACP pathway |
| 74490-74765c | mbcJ | methoxymalonyl ACP pathway |
| 74762-75628c* | mbck | methoxymalonyl ACP pathway |
| 75881-76537 | mbcG | methoxymalonyl ACP pathway |
| 76534-77802* | mbcP | C4,5 monooxygenase |
| 77831-79054* | mbcP450 | P450 |
| 79119-79934* | mbcMT1 | O-methyltransferase |
| 79931-80716* | mbcMT2 | O-methyltransferase |

[Note 1: c indicates that the gene is encoded by the complement DNA strand; Note 2: it is sometimes the case that more than one potential candidate start codon can been identified. One skilled in the art will recognise this and be able to identify alternative possible start codons. We have indicated those genes which have more than one possible start codon with a ‘*’ symbol. Throughout we have indicated what we believe to be the start codon, however, a person of skill in the art will appreciate that it may be possible to generate active protein using an alternative start codon.]

### Example 2

### Generation of Strain BIOT-3806: an Actinosynnema pretiosum Strain in which the gdmM Homologue mcbM has been Interrupted by Insertion of a Plasmid and Isolation of the C21-deoxymacbecin Analogues 17 and 18

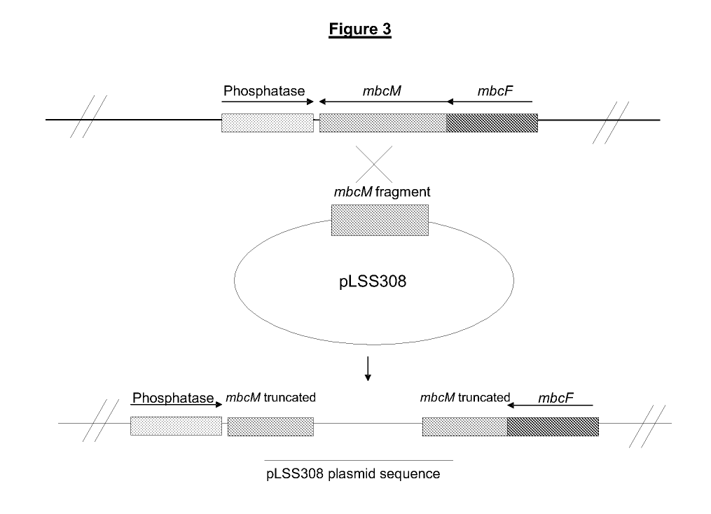

A summary of the construction of pLSS308 is shown in FIG. 3.

### 2.1. Construction of Plasmid pLSS308

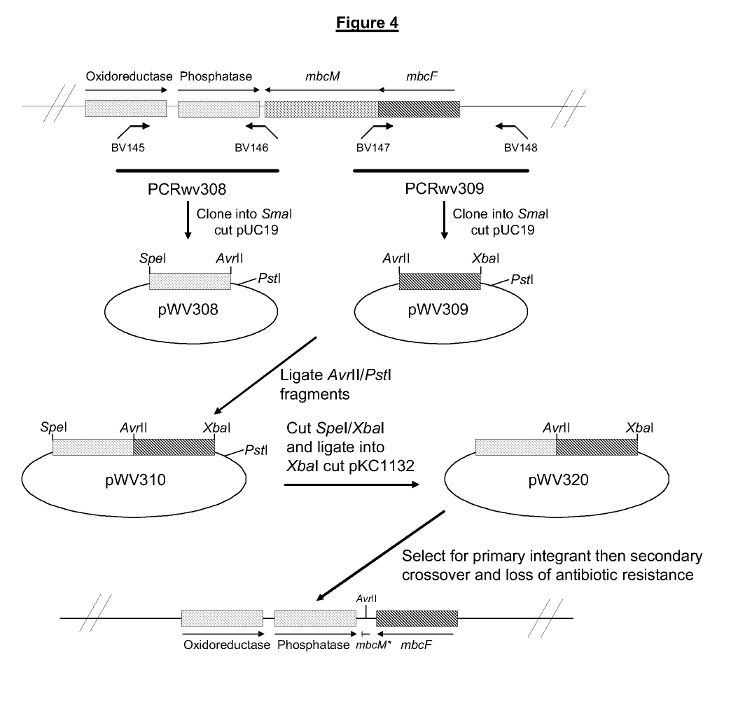

The DNA sequences of the gdmM gene from the geldanamycin biosynthetic gene cluster of Streptomyces hygroscopicus strain NRRL 3602 (AY179507) and orf19 from the rifamycin biosynthetic gene cluster of Amycolatopsis mediterranei (AF040570 AF040571) were aligned using Vector NTI sequence alignment program (FIG. 4). This alignment identified regions of homology that were suitable for the design of degenerate oligos that were used to amplify a fragment of the homologous gene from Actinosynnema mirum (BIOT-3134; DSM43827; ATCC29888). The degenerate oligos are:

(SEQ ID NO: 12)

| | |
|---|---|
| FPLS1: | 5′: ccscgggcgnycngsttcgacngygag 3′; |

(SEQ ID NO: 13)

| | |
|---|---|
| FPLS3: | 5′: cgtcncggannccggagcacatgccctg 3′; |

where n=G, A, T or C; y=C or T; s=G or C

The template for PCR amplification was Actinosynnema mirum cosmid 43. The generation of cosmid 43 is described in Example 1 above.

Oligos FPLS1 and FPLS3 were used to amplify the internal fragment of a gdmM homologue from Actinosynnema mirum in a standard PCR reaction using cosmid 43 as the template and Taq DNA polymerase. The resultant 793 bp PCR product was cloned into pUC19 that had been linearised with SmaI, resulting in plasmid pLSS301. The cloned region was sequenced and was shown to have significant homology to gdmM. An alignment of the gene fragment amplified from cosmid 43 (A. mirum) with the sequence of the mbcM gene of the macbecin biosynthetic gene cluster of Actinosynnema pretiosum subsp. pretiosum shows only 1 bp difference between these sequences (excluding the region dictated by the sequence of the degenerate oligos). It was postulated that the amplified sequence is from the mcbM gene of the macbecin cluster of A. mirum. Plasmid pLSS301 was digested with EcoRI/HindIII and the fragment cloned into plasmid pKC1132 (Bierman et al., 1992) that had been digested with EcoRI/HindIII. The resultant plasmid, designated pLSS308, is apramycin resistant and contains an internal fragment of the A. mirum mbcM gene.

### 2.2 Transformation of Actinosynnema pretiosum Subsp. pretiosum

Escherichia coli ET12567, harbouring the plasmid pUZ8002 was transformed with pLSS308 by electroporation to generate the E. coli donor strain for conjugation. This strain was used to transform Actinosynnema pretiosum subsp. pretiosum by vegetative conjugation (Matsushima et al., 1994). Exconjugants were plated on Medium 4 and incubated at 28° C. Plates were overlayed after 24 h with 50 mg/L apramycin and 25 mg/L nalidixic acid. As pLSS308 is unable to replicate in Actinosynnema pretiosum subsp. pretiosum, any apramycin resistant colonies were anticipated to be transformants that contained plasmid integrated into the mbcM gene of the chromosome by homologous recombination via the plasmid borne mcbM internal fragment (FIG. 3). This results in two truncated copies of the mbcM gene on the chromosome. Transformants were confirmed by PCR analysis and the amplified fragment was sequenced. Colonies were patched onto Medium 4 (with 50 mg/L apramycin and 25 mg/L nalidixic acid). A 6 mm circular plug from each patch was used to inoculate individual 50 mL falcon tubes containing 10 mL seed medium (variant of Medium 1—2% glucose, 3% soluble starch, 0.5% corn steep solids, 1% soybean flour, 0.5% peptone, 0.3% sodium chloride, 0.5% calcium carbonate) plus 50 mg/L apramycin. These seed cultures were incubated for 2 days at 28° C., 200 rpm with a 5 cm throw. These were then used to inoculate (5% v/v) fermentation medium (Medium 2) and were grown at 28° C. for 24 hours and then at 26° C. for a further 5 days. Metabolites were extracted from these according to the standard protocol described above. Samples were assessed for production of macbecin analogues by HPLC using the standard protocol described above.

The productive isolate selected was designated BIOT-3806.

### 2.3 Identification of Compounds from BIOT-3806

LCMS was performed using an Agilent HP1100 HPLC system in combination with a Bruker Daltonics Esquire 3000+ electrospray mass spectrometer operating in positive and/or negative ion mode. Chromatography was achieved over a Phenomenex Hyperclone column (C₁₈ BDS, 3 u, 150×4.6 mm) eluting at a flow rate of 1 mL/min using the following gradient elution process; T=0, 10% B; T=2, 10% B; T=20, 100% B; T=22, 100% B; T=22.05, 10% B; T=25, 10% B. Mobile phase A=water+0.1% formic acid; mobile phase B=acetonitrile+0.1% formic acid. UV spectra were recorded between 190 and 400 nm, with extracted chromatograms taken at 210, 254 and 276 nm. Mass spectra were recorded between 100 and 1500 amu.

TABLE 8
compounds identified by LCMS

| Compound | Retention time (min) | [M + Na]⁺ | [M − H]⁻ | Mass |
|---|---|---|---|---|
| 17 | 11.4 | 525.2 | 501.2 | 502 |
| 18 | 9.7 | 541.1 | 517.1 | 518 |

### 2.4 Fermentation and Isolation of 7-O-carbamoylpre-macbecin (17)

### Alternative Name 4,5-dihydro-11-O-demethyl-15-demethoxy-18,21-didehydro-21-deoxomacbecin

Vegetative stocks of BIOT-3806 were prepared after growth in Medium 1 with 50 mg/L apramycin, and preserved in 20% w/v glycerol: 10% w/v lactose in distilled water and stored at −80° C. Vegetative stocks were recovered onto plates of ISP2 medium (Medium 3) supplemented with 50 mg/L apramycin and incubated for 48 hours at 28° C. Vegetative cultures were prepared by removing two agar plugs, 5 mm in diameter from the ISP2 plate and inoculating them into 30 mL Medium 1 in 250 mL shake flasks containing 50 mg/L apramycin. The flasks were incubated at 28° C., 200 rpm (5 cm throw) for 48 h.

Vegetative cultures were inoculated at 5% v/v into 200 ml production medium (Medium 2) in 2 L shake flasks. Cultivation was carried out for 1 day at 28° C. followed by 5 days at 26° C., 300 rpm (2.5 cm throw).

The fermentation broth of BIOT-3806 (1 L, pink colour) was extracted three times with an equal volume of ethyl acetate (EtOAc). The solvent was removed from the combined EtOAc extract in vacuo to yield 2.34 g of brown oil. The extract was then chromatographed over Silica Gel 60 eluting initially with a CHCl₃ and MeOH mixture (95:5) followed by an increase in MeOH concentration up to 10% and collection of several fractions (approx. 250 mL per fraction). The fractions were assayed for the presence of metabolites using HPLC. A particular fraction containing a new compound (fraction 5; 334 mg crude mass after removal of solvent) was further purified by chromatography over a Phenomenex Luna C18-BDS column (21.2×250 mm; 5 um particle size) eluting with a gradient of water:acetonitrile (80:20) to (50:50) over a period of 25 min, with a flow rate of 21 mL/min. Fractions were assayed by analytical HPLC and those containing the new compound were combined and the solvents removed to yield an off white solid (86 mg). Analysis by LCMS/MS, and by 1D and 2D NMR experiments carried out in acetone-d₆ identified the compound as 7-O-carbamoylpre-macbecin (17)

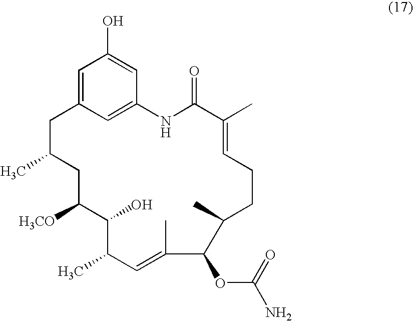

### 2.5 Fermentation and Isolation of 7-O-carbamoyl-15-hydroxypre-macbecin (18)

### Alternative Name 4,5-dihydro-11,15-O-didemethyl-18,21-didehydro-21-deoxomacbecin

Vegetative stocks of BIOT-3806 were prepared after growth in medium 1 containing 50 mg/L apramycin and preserved in 20% w/v glycerol: 10% w/v lactose in distilled water and stored at −80° C. Vegetative stocks were recovered onto plates of ISP2 medium (Medium 3) supplemented with 50 mg/L apramycin and incubated for 48 hours at 28° C. Vegetative cultures were prepared by removing two agar plugs, 5 mm in diameter, from the ISP2 plate and inoculating them into 30 mL Medium 1 in 250 mL shake flasks containing 50 mg/L apramycin. The flasks were incubated at 28° C., 200 rpm (5 cm throw) for 48 h.

Vegetative cultures were inoculated at 5% v/v into 200 mL production medium (medium 2) in 2 L shake flasks. Cultivation was carried out for 1 day at 28° C. followed by 5 days at 26° C., 200 rpm (5 cm throw).

The fermentation broth of BIOT-3806 (1.3 L, cream colour) was extracted three times with an equal volume of ethyl acetate (EtOAc). The solvent was removed from the combined extract in vacuo to yield 2.87 g of a brown oil. The extract was then chromatographed over Silica Gel 60 eluting initially with a CHCl₃ and MeOH mixture (95:5) followed by an increase in MeOH concentration up to 10% and collection of several fractions (about 250 mL per fraction). The fractions were assayed for the presence of metabolites using HPLC. A particular fraction containing a new compound (fraction 7; 752 mg crude mass after removal of solvent) was further purified by chromatography over a Phenomenex Luna C18-BDS column (21.2×250 mm; 5 um particle size) eluting with a gradient of water; acetonitrile (85:15) to (55:45) over 25 min, with a flow rate of 21 mL/min. Fractions were assayed by analytical HPLC and those containing the new compound were combined and the solvents removed to yield an off white solid (245.5 mg). For identification and characterisation MS, and 1 and 2D NMR experiments were carried out in Acetone-d₆. Analysis by LCMS/MS, and by 1D and 2D NMR carried out in acetone-d₆ identified the compound as 7-O-carbamoyl-15-hydroxypre-macbecin (18).

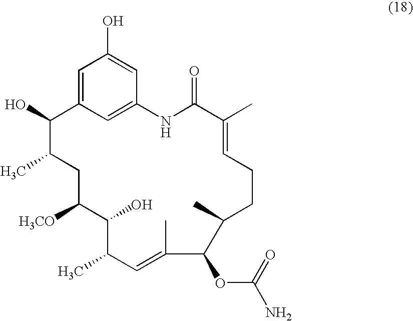

### 2.6 Identification and Characterisation:

A range of MS and NMR experiments were performed, viz LCMS, MSMS, 1H, 13C, APT, COSY-45, HMQC, HMBC. A thorough and exhaustive review of these data enabled the assignment of the majority of the protons and carbons of two analogues of pre-macbecin. The NMR assignments are described in Table 9.

TABLE 9

| | |
|---|---|
| | 17 |
| 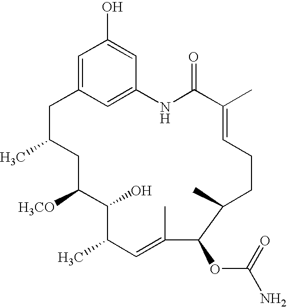 | |
| | 18 |
| 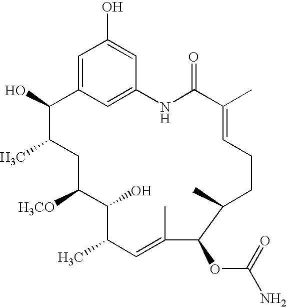 | |

| | ¹H-NMR | | ¹³C-NMR | |
|---|---|---|---|---|
| Position | 17 | 18 | 17 | 18 |
| 1 | — | — | 171.8 | 172.5 |
| 2 | — | — | 135.5 | 135.5 |
| 2-CH₃ | 1.82 s | 1.81 s | 14.0 | 14.3 |
| 3 | 6.17 bs | 6.02 s | 133.8 | 133.7* |
| 4 | 2.40 m | 2.34 m | 28.5* | 27.7* |
| | 2.19 m | 2.12*** | | |
| 5 | 1.46 m | 1.32 m | 33.6* | 36.1* |
| | 1.32 m | 1.21 m | | |
| 6 | 1.91 m | 1.84 m | 36.3 | 35.7 |
| 6-CH₃ | 0.87 d, 7 | 0.86 d, 7 | 16.4 | 16.4 |
| 7 | 5.17 br.s | 5.01 br.s | 81.9 | 82.1 |
| 7-CONH₂ | — | — | 159.0 | 159.5 |
| 8 | — | — | 134.0 | 134.5 |
| 8-CH₃ | 1.50 s | 1.44 s | 14.4 | 13.9 |
| 9 | 5.35 d, 9.5 | 5.29 d, 9.5 | 131.4 | 132.7 |
| 10 | 2.45 m | 2.42 m | 36.0 | 35.7 |
| 10-CH₃ | 1.01 d, 7 | 1.00 d, 7 | 18.6 | 18.8 |
| 11 | 3.60 dd, 8.5, 2.5 | 3.62 dd, 8.5, 2.5 | 76.3 | 75.9 |
| 12 | 3.18 ddd, 6, 3, 3 | 3.15 ddd, 6, 3, 3 | 84.1 | 83.4 |
| 12-OCH₃ | 3.30 s | 3.30 s | 57.6 | 57.5 |
| 13 | 1.55 m | 1.84** m | CA | 32.9 |
| | 1.34 m | | | |
| 14 | 1.63 m | 1.84** m | 36.7 | 40.5 |
| 14-CH₃ | 0.85 d, 7 | 0.75 d, 6.5 | 21.3 | 15.9 |
| 15 | 2.66 dd, 12, 1.5 | 4.62 d, 1.5 | 43.9 | 76.5 |
| | 2.13 m | | | |
| 15-OH | — | — | — | — |
| 16 | — | — | 144.9 | 141.9 |
| 17 | 6.36 s | 6.32 s | 113.5 | 111.8 |
| 18 | — | — | 159.3 | 158.5 |
| 18-OH | 8.22 br.s | 8.38 br.s | — | — |
| 19 | 7.34 bs | 7.16 s | 106.1 | 107.2 |
| 20 | — | — | 142.6 | 148.1 |
| 21 | 6.41 s | 6.76 s | 114.6 | 110.6 |

* connectivities for these carbons could not be made and assignments given are based on similarity to related molecules;
CA, this carbon could not be assigned;
**COSY correlations clearly distinguish these different signals;
***only observed as COSY cross peak.

### Example 3

### Generation of an Actinosynnema pretiosum Strain in which the gdmM Homologue mbcM has an In-Frame Deletion and Production of the C21-desoxy Macbecin Analogues 17 and 18

### 3.1 Cloning of DNA Homologous to the Downstream Flanking Region of mbcM

Oligos BV145 (SEQ ID NO: 14) and BV146 (SEQ ID NO: 15) were used to amplify a 1421 bp region of DNA from Actinosynnema pretiosum (ATCC 31280) in a standard PCR reaction using cosmid 52 (from example 1) as the template and Pfu DNA polymerase. A 5′ extension was designed in each oligo to introduce restriction sites to aid cloning of the amplified fragment (FIG. 4). The amplified PCR product encoded 33 bp of the 3′ end of mbcM and a further 1368 bp of downstream homology. This 1421 bp fragment was cloned into pUC19 that had been linearised with SmaI, resulting in plasmid pWV308.

### 3.2 Cloning of DNA Homologous to the Upstream Flanking Region of mbcM

Oligos BV147 (SEQ ID NO: 16) and BV148 (SEQ ID NO: 17) were used to amplify a 1423 bp region of DNA from Actinosynnema pretiosum (ATCC 31280) in a standard PCR reaction using cosmid 52 (from example 1) as the template and Pfu DNA polymerase. A 5′ extension was designed in each oligo to introduce restriction sites to aid cloning of the amplified fragment (FIG. 4). The amplified PCR product encoded 30 bp of the 5′ end of mbcM and a further 1373 bp of upstream homology. This 1423 bp fragment was cloned into pUC19 that had been linearised with SmaI, resulting in plasmid pWV309.

BV145

(SEQ ID NO: 14)

ATATACTAGTCACGTCACCGGCGCGGTGTCCGCGGACTTCGTCAACG
SpeI
BV146

(SEQ ID NO: 15)

ATATCCTAGGCTGGTGGCGGACCTGCGCGCGCGGTTGGGGTG
AvrII
BV147

(SEQ ID NO: 16)

ATATCCTAGGCACCACGTCGTGCTCGACCTCGCCCGCCACGC
AvrII
BV148

(SEQ ID NO: 17)

ATATTCTAGACGCTGTTCGACGCGGGCGCGGTCACCACGGGC
XbaI

The products PCRwv308 and PCRwv309 were cloned into pUC19 in the same orientation to utilise the PstI site in the pUC19 polylinker for the next cloning step.

The 1443 bp AvrII/PstI fragment from pWV309 was cloned into the 4073 bp AvrII/PstI fragment of pWV308 to make pWV310. pWV310 therefore contained a SpeI/XbaI fragment encoding DNA homologous to the flanking regions of mbcM fused at an AvrII site. This 2816 bp SpeI/XbaI fragment was cloned into pKC1132 (Bierman et al., 1992) that had been linearised with SpeI to create pWV320.

### 3.3 Transformation of Actinosynnema pretiosum subsp. pretiosum

Escherichia coli ET12567, harbouring the plasmid pUZ8002 was transformed with pWV320 by electroporation to generate the E. coli donor strain for conjugation. This strain was used to transform Actinosynnema pretiosum subsp. pretiosum by vegetative conjugation (Matsushima et al, 1994). Exconjugants were plated on Medium 4 and incubated at 28° C. Plates were overlayed after 24 h with 50 mg/L apramycin and 25 mg/L nalidixic acid. As pWV320 is unable to replicate in Actinosynnema pretiosum subsp. pretiosum, apramycin resistant colonies were anticipated to be transformants that contained plasmid pWV320 integrated into the chromosome by homologous recombination via one of the plasmid borne mbcM flanking regions of homology.

Genomic DNA was isolated from six exconjugants and was digested and analysed by Southern blot. The blot showed that in four out of the six isolates integration had occurred in the upstream region of homology and in two of the six isolates homologous integration had occurred in the downstream region. One strain resulting from homologous integration in the upstream region (designated BIOT-3831) was chosen for screening for secondary recombinants. One strain resulting from homologous integration in the downstream region (BIOT-3832) was also chosen for screening for secondary recombinants.

### 3.4 Screening for Secondary Recombinants

Strains were patched onto medium 4 (supplemented with 50 mg/L apramycin) and grown at 28° C. for four days. A 1 cm² section of each patch was used to inoculate 7 mL modified ISP2 (0.4% yeast extract, 1% malt extract, 0.4% dextrose in 1 L distilled water) without antibiotic in a 50 mL falcon tube. Cultures were grown for 2-3 days then subcultured on (5% inoculum) into another 7 mL modified ISP2 (see above) in a 50 mL falcon tube. After 4-5 generations of subculturing the cultures were sonicated, serially diluted, plated on Medium 4 and incubated at 28° C. for four days. Single colonies were then patched in duplicate onto Medium 4 containing apramycin and onto Medium 4 containing no antibiotic and the plates were incubated at 28° C. for four days. Patches that grew on the no antibiotic plate but did not grow on the apramycin plate were re-patched onto +/− apramycin plates to confirm that they had lost the antibiotic marker. Genomic DNA was isolated from all apramycin sensitive strains and analysed by Southern blot. At this stage, half the secondary crossover strains had reverted to wild-type but half had produced the desired mbcM deletion mutants. The mutant strain encodes an mbcM protein with an in-frame deletion of 502 amino acids (FIG. 9).

mbcM deletion mutants were patched onto Medium 4 and grown at 28° C. for four days. A 6 mm circular plug from each patch was used to inoculate individual 50 mL falcon tubes containing 10 mL seed medium (adapted from medium 1—2% glucose, 3% soluble starch, 0.5% corn steep solids, 1% soybean flour, 0.5% peptone, 0.3% sodium chloride, 0.5% calcium carbonate). These seed cultures were incubated for 2 days at 28° C., 200 rpm with a 2 inch throw. These were then used to inoculate (0.5 mL into 10 mL) production medium (medium 2-5% glycerol, 1% corn steep solids, 2% yeast extract, 2% potassium dihydrogen phosphate, 0.5% magnesium chloride, 0.1% calcium carbonate) and were grown at 28° C. for 24 hours and then at 26° C. for a further 5 days. Secondary metabolites were extracted from these cultures by the addition of an equal volume of ethyl acetate. Cell debris was removed by centrifugation. The supernatant was transferred to a clean tube and solvent was removed in vacuo. Samples were reconstituted in 0.23 mL methanol followed by the addition of 0.02 mL of 1% (w/v) FeCl₃ solution. Samples were assessed for production of macbecin analogues

Chemical analysis by LCMS using the methods described in example 2.3 above unambiguously identified the presence of compounds 18 and 19 based on them having identical retention times and mass spectra.

### 3.5 Selection of Individual Colonies by Generating Protoplasts of BIOT-3872

Protoplasts were generated from BIOT-3872 using a method adapted from Weber and Losick 1988 with the following media alterations; Actinosynnema pretiosum cultures were grown on ISP2 plates (medium 3) for 3 days at 28° C. and a 5 mm² scraping used to inoculate 40 mL of ISP2 broth supplemented with 2 mL of sterile 10% (w/v) glycine in water. Protoplasts were generated as described in Weber and Losick 1988 and then regenerated on R2 plates (R2 recipe—Sucrose 103 g, K₂SO₄ 0.25 g, MgCl₂.6H₂O 10.12 g, Glucose 10 g, Difco Casaminoacids 0.1 g, Difco Bacto agar 22 g, distilled water to 800 mL, the mixture was sterilised by autoclaving at 121° C. for 20 minutes. After autoclaving the following autoclaved solutions were added; 0.5% KH₂PO₄ 10 mL, 3.68% CaCl₂.2H₂O 80 mL, 20% L-proline 15 mL, 5.73% TES buffer (pH7.2) 100 mL, Trace element solution (ZnCl₂ 40 mg, FeCl₃.6H₂O 200 mg, CuCl₂.2H₂O 10 mg, MnCl₂.4H₂O 10 mg, Na₂B₄O₇.10H₂O 10 mg, (NH₄)₆Mo₇O₂₄.4H₂O 10 mg, distilled water to 1 litre) 2 mL, NaOH (1 N) (unsterilised) 5 mL).

80 individual colonies were patched onto MAM plates (Medium 4) and analysed for production of macbecin analogues as described above in example 2.3. The majority of protoplast generated patches produced at similar levels to the parental strain. 15 out of the 80 samples tested produced significantly more 18 and 19 than the parental strain. The best producing strain, WV4a-33 (BIOT-3870) was observed to produce 18 and 19 at significantly higher levels than the parent strain.

### Example 4

### Generation of an Actinosynnema pretiosum Strain in which mbcM has an In-Frame Deletion and mbcMT1, mbcMT2, mbcP and mbcP450 have Additionally been Deleted and Production of the C-21 Desoxy Macbecin Analogue 17

### 4.1 Cloning of DNA Homologous to the Downstream Flanking Region of mbcMT2

Oligos Is4del1 (SEQ ID NO: 18) and Is4del2a (SEQ ID NO: 19) were used to amplify a 1595 bp region of DNA from Actinosynnema pretiosum (ATCC 31280) in a standard PCR reaction using cosmid 52 (from example 1) as the template and Pfu DNA polymerase. A 5′ extension was designed in oligo Is4del2a to introduce an AvrII site to aid cloning of the amplified fragment (FIG. 10). The amplified PCR product encoded 196 bp of the 3′ end of mbcMT2 and a further 1393 bp of downstream homology. This 1595 bp fragment was cloned into pUC19 that had been linearised with SmaI, resulting in plasmid pLSS1+2a.

(SEQ ID NO: 18)

| | |
|---|---|
| Is4del1 | 5′-GGTCACTGGCCGAAGCGCACGGTGTCATGG-3′ |

(SEQ ID NO: 19)

| | |
|---|---|
| Is4del2a | 5′-CCTAGGCGACTACCCCGCACTACTACACCGAGCAGG-3′ |

### 4.2 Cloning of DNA Homologous to the Upstream Flanking Region of mbcM

Oligos Is4del3b (SEQ ID NO: 20) and Is4del4 (SEQ ID NO: 21) were used to amplify a 1541 bp region of DNA from Actinosynnema pretiosum (ATCC 31280) in a standard PCR reaction using cosmid 52 (from example 1) as the template and Pfu DNA polymerase. A 5′ extension was designed in oligo Is4del3b to introduce an AvrII site to aid cloning of the amplified fragment (FIG. 10). The amplified PCR product encoded ˜100 bp of the 5′ end of mbcP and a further ˜1450 bp of upstream homology. This ˜1550 bp fragment was cloned into pUC19 that had been linearised with SmaI, resulting in plasmid pLSS3b+4.

(SEQ ID NO: 20)

| | |
|---|---|
| Is4del3b | 5′-CCTAGGAACGGGTAGGCGGGCAGGTCGGTG-3′ |

(SEQ ID NO: 21)

| | |
|---|---|
| Is4del4 | 5′-GTGTGCGGGCCAGCTCGCCCAGCACGCCCAC-3′ |

The products 1+2a and 3b+4 were cloned into pUC19 to utilise the HindIII and BamHI sites in the pUC19 polylinker for the next cloning step.

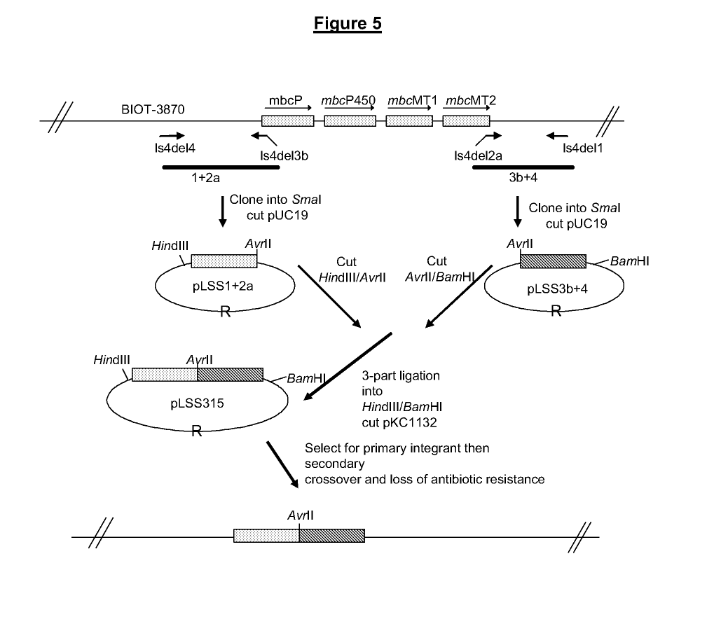

The 1621 bp AvrII/HindIII fragment from pLSS1+2a and the 1543 bp AvrII/BamHI fragment from pLSS3b+4 were cloned into the 3556 bp HindIII/BamHI fragment of pKC1132 to make pLSS315. pLSS315 therefore contained a HindIII/BamHI fragment encoding DNA homologous to the flanking regions of the desired four ORF deletion region fused at an AvrII site (FIG. 5).

### 4.3 Transformation of BIOT-3870 with pLSS315

Escherichia coli ET12567, harbouring the plasmid pUZ8002 was transformed with pLSS315 by electroporation to generate the E. coli donor strain for conjugation. This strain was used to transform BIOT-3870 by vegetative conjugation (Matsushima et al, 1994). Exconjugants were plated on MAM medium (1% wheat starch, 0.25% corn steep solids, 0.3% yeast extract, 0.3% calcium carbonate, 0.03% iron sulphate, 2% agar) and incubated at 28° C. Plates were overlayed after 24 h with 50 mg/L apramycin and 25 mg/L nalidixic acid. As pLSS315 is unable to replicate in BIOT-3870, apramycin resistant colonies were anticipated to be transformants that contained plasmid integrated into the chromosome by homologous recombination via the plasmid borne regions of homology.

### 4.4 Screening for Secondary Recombinants

Three primary transformants of BIOT-3870:pLSS315 were selected for subculturing to screen for secondary recombinants.

Strains were patched onto MAM media (supplemented with 50 mg/L apramycin) and grown at 28° C. for four days. Two 6 mm circular plugs were used to inoculate 30 mL of ISP2 (0.4% yeast extract, 1% malt extract, 0.4% dextrose, not supplemented with antibiotic) in a 250 ml conical flask. Cultures were grown for 2-3 days then subcultured (5% inoculum) into 30 mL of ISP2 in a 250 ml conical flask. After 4-5 rounds of subculturing the cultures were protoplasted as described in Example 3.6, the protoplasts were serially diluted, plated on regeneration media (see Example 3.6) and incubated at 28° C. for four days. Single colonies were then patched in duplicate onto MAM media containing apramycin and onto MAM media containing no antibiotic and the plates were incubated at 28° C. for four days. Seven patches derived from clone no 1 (no 32-37) and four patches derived from clone no 3 (no 38-41) that grew on the no antibiotic plate but did not grow on the apramycin plate were re-patched onto +/− apramycin plates to confirm that they had lost the antibiotic marker.

Production of macbecin analogues was carried out as described in the General Methods. Analysis was performed as described in General Methods and example 2. Compound 17 was produced in yields comparable to the parent strain BIOT-3870 and no production of compound 18 was observed for patches 33, 34, 35, 37, 39 and 41. This result shows that the desired mutant strains have a deletion of 3892 bp of the macbecin cluster containing the genes mbcP, mbcP450, mbcMT1 and mbcMT2 in addition to the original deletion of mbcM.

### Example 5

### Synthesis of 4,5-dihydro-11-O-demethyl-15-demethoxy-18,21-didehydro-21-deoxomacbecin-18-phosphate (Compound 20)

Under inert conditions 4,5-dihydro-11-O-demethyl-15-demethoxy-18,21-didehydro-21-deoxomacbecin (110 mg, 2.19×10−4 mol, 1 equivalent) was dissolved in tetrahydrofuran (5 ml, 44 mM soln) and cooled in an water ice bath. Triethylamine (0.185 ml, 1.31×10−3 mol, 6 equivalents) was added followed by the dropwise addition from a microsyringe of phosphoryl chloride (0.03 ml, 3.28×10−4 mol, 1.5 equivalents). The reaction was left to stir, on water ice, under inert conditions for a further 1 hour. At that time water (0.5 ml, 28 mmol, 125 equivalents) was added and the reaction stirred for a further 1 hour on water ice. The solvent was then removed in vacuo, to yield a white solid.

Sephadex G25 was left to swell overnight in HPLC grade water and then a column prepared (2.5 cm diameter×40 cm). The white solid was dissolved in water (5 ml) and acetonitrile (1 ml) and added to the column, which was eluted with water. 10 ml fractions were collected and those containing a UV active compound were pooled and taken to dryness, to yield a white solid (240 mg).

The material was further desalted over a BioRad P2 column, eluted with water and the UV active fractions pooled and taken to dryness.

A portion of this material (11.6 mg) was dissolved in water (3 ml) and added, under gravity, to 20 g of DOWEX 50Wx8 200 (in Na⁺ cycle) then eluted with HPLC grade water. The UV active fractions were pooled and taken to dryness (6.5 mg).

LC-MS (method as described in section 2.3): RT 8.8 minutes, positive ion (m/z)=605.5 ([M+Na]⁺ adduct), negative ion (m/z)=581.5 ([M−H]⁻)

¹H NMR, D₂O (referenced to 4.60 ppm) 400 MHz, chemical shifts include, (ppm): 6.65 (s), 6.57 (s), 6.39 (s), 5.57 (bm), 4.95 (d, J=10 Hz), 4.29 (bd, J=7 Hz), 3.47 (bd, J=10 Hz), 3.08 (3H, s), 2.90 (bd, J=10 Hz), 2.53 (m), 2.23 (m), 2.03 (m), 1.83 (m), 1.62 (m), 1.67 (m), 1.58 (3H, s), 1.53 (m), 1.32 (m), 1.08 (s), 1.01 (m), 0.86 (m), 0.76 (3H, d, J=6 Hz), 0.61 (3H, d, J=6.5 Hz), 0.50 (3H, d, J=6.5 Hz)

### Example 6

### Synthesis of 18-O-(N,N′-dimethylpropanediamine carbamoyl)-4,5-dihydro-11-O-demethyl-15-demethoxy-18,21-didehydro-18-hydroxy-21-deoxomacbecin (Compound 21)

### 6.1 Synthesis of 18-O-(4-nitrophenylcarbonate)-4,5-dihydro-11-O-demethyl-15-demethoxy-18,21-didehydro-21-deoxomacbecin

Under inert conditions 4,5-dihydro-11-O-demethyl-15-demethoxy-18,21-didehydro-21-deoxomacbecin (260 mg, 0.517 mmol, 1 equivalent) was dissolved in dichloromethane (20 ml). 4-Nitrophenylchloroformate (130 mg, 0.646 mmol, 1.25 equivalents) was dissolved in dichloromethane (1 ml) and added to the 21-desoxoansamycin solution. The reaction was then heated under reflux and 2,6-lutidine (0.078 ml, 0.672 mmol, 1.30 equivalents) added. After 3 hours heating under reflux the reaction was diluted to 50 ml with further dichloromethane and washed with 1 M HCl aq. (2×50 ml) and water (2×50 ml) and the organics dried over sodium sulfate and reduced in vacuo to yield an off-white solid (420 mg). This material was purified over a sephadex LH20 column. Sephadex LH20 had been swollen overnight in methanol/dichloromethane (1:1), and a column prepared (3 cm diameter×90 cm). The material was eluted from the column in methanol/dichloromethane (1:1), collecting 3 ml fractions. Fractions 60-69 contained the desired product and were pooled and taken to dryness (188 mg, off-white solid). This was further purified by preparative HPLC (Phenomenex, LUNA C18, 25 cm×22.5 mm diameter, running 21 ml/min from 50% solvent B to 80% solvent B over 30 minutes. Solvent A is water, solvent B is acetonitrile) in 3 separate injections. The combined fractions were pooled and taken to dryness in vacuo to yield the desired compound (93.3 mg, off-white solid, 1.39×10−4 mol, 27%).

LCMS (method described in general synthesis section above): RT: 7.7 minutes, positive ion (m/z)=690.3 ([M+Na]⁺), negative ion (m/z)=666.5 ([M−H]⁻), 712.4 (M+HCOO⁻)

### 6.2 Preparation of 18-O-(N,N′-dimethylpropanediamine carbamoyl)-4,5-dihydro-11-O-demethyl-15-demethoxy-18,21-didehydro-18-hydroxy-21-deoxomacbecin

Under inert conditions 18-O-(4-nitrophenylcarbonate)-4,5-dihydro-11-O-demethyl-15-demethoxy-18,21-didehydro-21-deoxomacbecin (74 mg, 0.111 mmol) was dissolved in dichloromethane (2 ml). N-trityl-N,N′-dimethylpropanediamine (110 mg, 0.333 mmol, 3 equivalents) was dissolved in dichloromethane (2 ml) and added to the 21-desoxoansamycin derivative. The resultant solution was heated under reflux for 5 hours and then stirred at room temperature overnight, then the solvent was removed in vacuo and the desired compound purified over a sephadex LH20 column eluted with methanol/dichloromethane (1:1).

LCMS (method described in general synthesis section above): RT: 6-8 minutes, positive ion (m/z)=631.7 ([M-trityl+H]⁺), negative ion (m/z)=629.6 ([M-trityl−H]⁻), 675.5 (M-trityl+HCOO⁻). Broad peak on chromatogram due to the removal of the trityl group in the LC solvent conditions. Furthermore, parent compound 17 observed by LCMS due to cyclisation-release.

The trityl group is then removed using 2M HCl in ether.

### Example 7

### Synthesis of 18-O-(N,N′-diethylethylenediamine carbamoyl)-4,5-dihydro-11-O-demethyl-15-demethoxy-18,21-didehydro-18-hydroxy-21-deoxomacbecin, Compound 22

Under inert conditions 18-O-(4-nitrophenylcarbonate)-4,5-dihydro-11-O-demethyl-15-demethoxy-18,21-didehydro-21-deoxomacbecin (20 mg, 0.03 mmol) (synthesised as in example 6.1) was dissolved in dichloromethane (1 ml). N-trityl-N,N′-diethylethylenediamine (32 mg, 0.09 mmol, 3 equivalents) was dissolved in dichloromethane (1 ml) and added to the 21-desoxoansamycin derivative. The resultant solution was heated under reflux for 5 hours and then stirred at room temperature overnight, then the solvent was removed in vacuo and the desired compound purified over a sephadex LH20 column eluted with methanol/dichloromethane (1:1).

LCMS (method described in general synthesis section above): RT: 6-8 minutes, positive ion (m/z)=645.7 ([M-trityl+H]⁺), negative ion (m/z)=643.6 ([M-trityl−H]⁻), 689.6 (M-trityl+HCOO⁻). Broad peak on chromatogram due to the removal of the trityl group in the LC solvent conditions. Furthermore, parent compound 17 was observed by LCMS due to cyclisation-release.

The trityl group is then removed using 2M HCl in ether.

### Example 8

### Synthesis of 18-O-(N,N′-dimethylethylenediamine carbamoyl)-4,5-dihydro-11-O-demethyl-15-demethoxy-18,21-didehydro-18-hydroxy-21-deoxomacbecin, Compound 23

Under inert conditions 18-O-(4-nitrophenylcarbonate)-4,5-dihydro-11-O-demethyl-15-demethoxy-18,21-didehydro-21-deoxomacbecin (152 mg, 0.228 mmol) (synthesised as in example 6.1) was dissolved in dichloromethane (10 ml). N-trityl-N,N′-dimethylethyldiamine (276 mg, 0.835 mmol, 3.7 equivalents) was dissolved in dichloromethane (1 ml) and added to the 21-desoxoansamycin derivative. The resultant solution was heated under reflux for 5 hours and then stirred at room temperature overnight, then the solvent was removed in vacuo and the desired compound purified by normal phase column chromatography over silica eluted with 4:6 then 1:1 acetone/hexanes. The active fractions were combined and dried under reduced pressure to yield a white solid (187 mg, 0.218 mmol, 95% isolated yield).

¹³C NMR, d₆-acetone, 125 MHz, chemical shifts include, (ppm): 171.5, 159.1, 156.0, 153.6, 144.7, 142.1, 135.8, 133.7, 131.2, 129.3, 127.8, 119.6, 112.4, 84.3, 79.8, 76.5, 57.6, 53.6, 52.6, 48.9, 48.8, 43.5, 38.8, 37.8, 35.9, 33.2, 24.2, 16.4, 15.2, 12.6.

The trityl protected title compound (187 mg, 0.218 mmol) was dissolved in dichloromethane (80 ml) and cooled on salt/ice bath (approx −5 deg C.). 2M HCl in diethyl ether (0.2 ml, 0.4 mmol, 1.83 equivalents) was dissolved in DCM (1 ml) and added to the cooled solution over 30 seconds. After 5 minutes the vessel was allowed to warm to room temperature and stirred for a further 75 minutes. Hexane (150 ml) was added and the stirring stopped. A white precipitate formed that was allowed to settle over 30 minutes. The solvent was decanted and the resultant solid titrerated with ice cold hexane/diethyl ether (1:1 ml, 2×1 ml), to yield a white amorphous solid (80.6 mg, 57% isolated yield).

LCMS (method described in general synthesis section above): RT: 4.0 minutes, positive ion (m/z)=617.9 ([M+H]⁺), negative ion (m/z)=615.8 ([M−H]⁻), 661.8 (MαHCOO⁻)

### Example 9

### Solubility Assay

To analyse the solubilities of 17-AAG, geldanamycin, 18,21-dihydromacbecin and macbecin, solutions (25 mM) of the test compounds were prepared by dissolving 3-5 mg aliquots in the appropriate amount of DMSO.

Aliquots (0.01 mL) were added to 0.490 mL of pH 7.3 PBS in glass vials. For each time point, 3 PBS vials were prepared in amber glass vials. For the six hour time point triplicate aliquots in DMSO were also prepared.

The resulting 0.5 mM solutions were shaken for up to six hours, with vials removed for analysis at 1, 3 and 6 hours. Samples were analysed by HPLC (0.025 mL injections). Compounds were quantified by peak area measurement at 274 nm.

Solubility in 2% DMSO in PBS at each time point was determined by comparing total peak areas for each chromatogram with mean total peak area for chromatograms produced from the corresponding 6 hour DMSO solutions. (Mean total peak area in DMSO solutions was assumed to be equivalent to a 0.5 mM solution). The results are shown below in Table 8. The solubility of compound 17 was measured by a thermodynamic assay as described in the general methods.

It is not possible to measure the solubility of 20 and 23 by this method due to their intrinsic high aqueous solubility. The absolute limit of aqueous solubility of these compounds has not been tested, however compound 20 is completely soluble in deionised water at 10 mg/ml (17 mM) and compound 23 is completely soluble in aqueous 5 mM citrate, at pH 4.5, at 3 mg/ml (4.6 mM).

TABLE 8
Solubility Results

| | Solubility |
|---|---|
| | (mM) |
| macbecin | 0.081 |
| 18,21-dihydromacbecin | 0.136 |
| Geldanamycin | 0.0017 |
| 17-AAG | 0.171 |
| 17 | 0.716 |
| 20 | >17 |
| 23 | >4.6 |

Even without taking the solubilities of 20 and 23 to their limit, it can be seen that they are substantially more soluble than standard compounds such as 17-AAG, macbecin, geldanamycin, and the parent compound 17, and are therefore much easier to formulate (for example, they can be dissolved directly in water at useful concentrations for dosing).

### Example 10

### In Vitro Cleavage Assays

0.1 mg/ml Compound 23 was dissolved in aqueous 0.1 M potassium phosphate buffer at a specified pH, less than 3 minutes before the first injection. Samples were monitored by LC-UV, on an Agilent 1100 HPLC. Chromatography was achieved over a Phenomenex Hyperclone column, BDS C₁₈ 3 u (150×4.6 mm):

Mobile phase A: 0.1% Formic acid in water
Mobile phase B: 0.1% Formic acid in acetonitrile
Flow rate: 1 mL/minute:
Gradient, t=0 mins, B=10%; t=2 mins, B=10%; t=20 mins, B=100%.

Compound 23 elutes at 12.1 mins and compound 17 elutes at 11.2 minutes in these conditions.

0.05 ml injections were made, repeatedly from the same sample every 3.18 hours, for 50 hours. the ambient temperature was 23 degrees C. The UV response was measured for each time point for compound 23 and 17. The half-life of disappearance of compound 23 was calculated thus:

the natural logarithm of the response was plotted with respect to time (in hours). The slope and fit of the resultant line was calculated and the half life, t_{1/2}=(In 2)/lambda. Where lambda is the slope of the graph.

TABLE 9
in vitro cleavage data

| pH of phosphate buffer (or other vehicle) | compound 23 half-life (hours) |
|---|---|
| 7.4 | 2.0 |
| 6.5 | 11.9 |
| 5.9 | 69.3 |
| 4.5 | not calculable (zero slope) |
| 5% glucose | 32.0 |

The product of compound 23 cleavage was shown to be compound 17 by LC-UV-MS. Therefore, it can be seen from the data that 23 cleaves in vitro, at different rates depending on pH, to generate the active metabolite 17, and would therefore be anticipated to act as a prodrug in vivo.

### Example 11

### In Vitro Blood Cleavage Assays with Compound 20

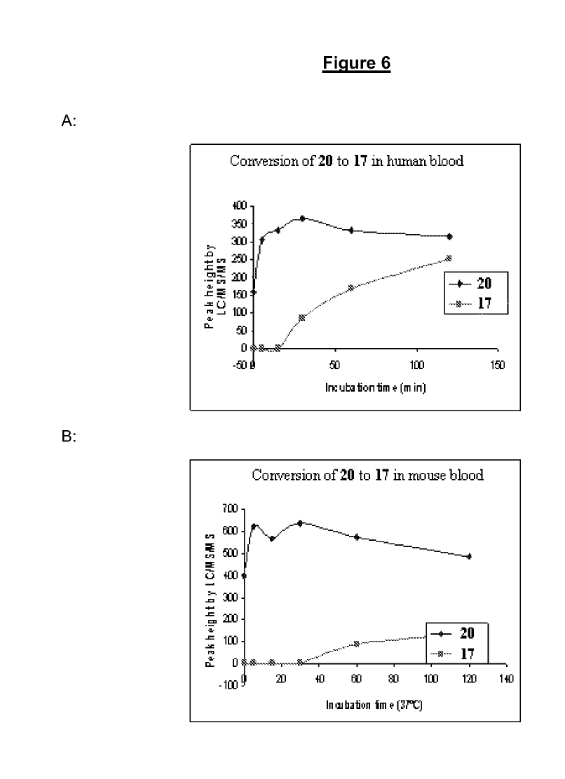

Blood cleavage assays were run to confirm that 20 acts as a prodrug and cleaves to produce compound 17 when incubated with human and mouse blood, as described in the general methods. The compound was incubated in human or mouse blood with samples removed over two hours, and analysed by LC MS/MS for presence of 20 and 17 (see FIG. 6). In both cases, 20 was seen to act as a prodrug and release 17. Due to the differing responses of 17 and 20 on mass spectrometry it was not possible to quantify the extent of conversion of 20 to 17.

### Example 12

### In Vitro Permeability Assays

The in vitro permeability of 20 and the parent 17 were assayed using caco-2 cells, as described in the general methods. All compounds were analysed at a concentration of 10 uM. The data generated in shown in table 9.

TABLE 10
Caco-2 permeability test results

| | Permeability (10⁻⁶ cm/s) | | | | | | | |
|---|---|---|---|---|---|---|---|---|
| | Apical to Basolateral | | | Basolateral to Apical | | | | |
| | Pₐₚₚ(A → B) | | | | Pₐₚₚ(B → A) | | | Efflux |
| Compound | Rep. 1 | Rep. 2 | Mean | Rep. 1 | Rep. 2 | Mean | $\frac{{P}_{app} ( B -> A )}{{P}_{app} ( A -> B )}$ | Limited ^{(A)} |
| 17 | 0.39 | 0.49 | 0.44 | 1.70 | 4.21 | 2.95 | 6.8 | Yes |
| 20 | 0.67 | 0.31 | 0.49 | 0.22 | 0.48 | 0.35 | 0.7 | No |

^{(A)} Efflux-limited permeability criteria:
Pₐₚₚ (B → A)/Pₐₚₚ (A → B) ≧ 3, and Pₐₚₚ (B → A) ≧ 1.0 × 10⁻⁶ cm/s

As can be seen, comparing 17 and its phosphate prodrug 20, whilst the absorption potential has remained similar (A to B), the reverse transport is far reduced, suggesting that the compound is now no longer efflux limited, possibly due to decreased effect from efflux transporters, such as P-gp. This might be expected to lead to improved bioavailability.

### Example 12

### Biological Data—In Vitro Evaluation of Anticancer Activity of Macbecin Analogues

In vitro evaluation of the test compound, 20, for anticancer activity in a panel of human tumour cell lines in a monolayer proliferation assay was carried out as described in the general methods using a modified propidium iodide assay.

The results are displayed in Table 6 below, all treated/control (% T/C) values shown are the average of 2 separate experiments. Table 12 shows the mean IC₇₀ for the compounds across the cell line panel tested, with macbecin shown as a reference (where the mean is calculated as the geometric mean of all replicates).

TABLE 11
in vitro cell line data

| | Test/Control (%) at drug | | | | |
|---|---|---|---|---|---|
| | concentration (μg/mL) | | | | |
| | Compound 20 | | | | |
| Cell line | 0.01 | 0.1 | 1 | 10 | 100 |
| CNXF 498NL | 99 | 77 | 15 | 16 | 12 |
| CXF HT29 | 112 | 43 | 7 | 7 | 5 |
| LXF 1121L | 103 | 58 | 15 | 14 | 5 |
| MCF-7 | 98 | 38 | 14 | 13 | 9 |
| MEXF 394NL | 102 | 50 | 17 | 16 | 3 |
| DU145 | 99 | 53 | 8 | 9 | 3 |

TABLE 12
average IC₇₀ value across the cell-line panel

| | IC₇₀ |
|---|---|
| | (μg/mL) |
| macbecin | 0.21 |
| 20 | 0.344 |

The potency of 20 is therefore in a similar range to macbecin, although it is unknown whether this is due to inherent activity of the compound, or due to cleavage and release of the parent. LC-MS analysis of the supernatant of the cell cultures revealed the presence of low amounts of 17, but not 20. The presence of 17 may be due to release into the supernatent following cleavage from 20 to 17 in the cells.

### Example 13

### Biological Data—In Vivo Pharmacokinetic Assay

In vivo confirmation of the cleavage of 20 to the parent molecule, 17, was carried out as described in the general methods.

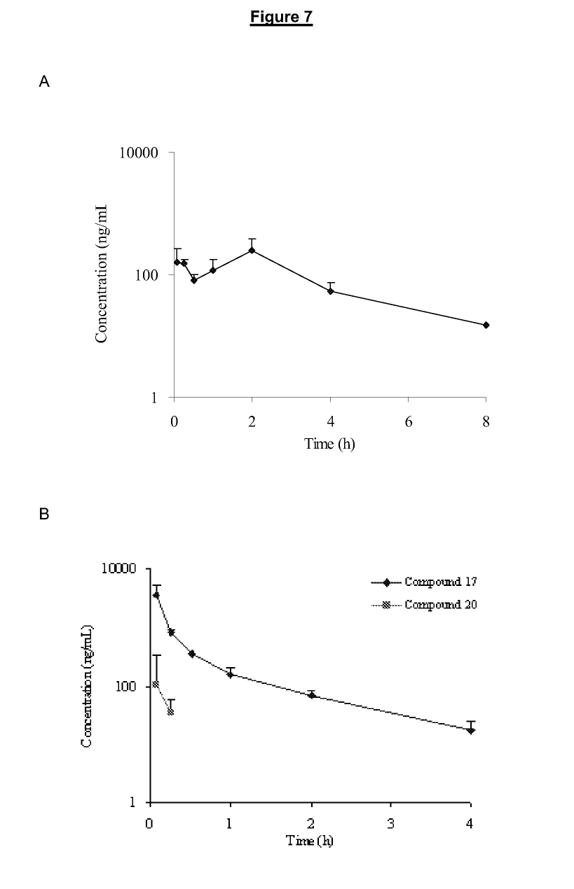

Female CD-1 mice were given single doses of 20, either intravenously at 3 mg/kg, or orally at 10 mg/kg. Plasma samples were then collected at 8 time points (0.067, 0.25, 0.5, 1, 2, 4, 8 and 24 h postdose) over a 24-h period. The amounts of 20 and 17 in the samples were then analysed. FIG. 7 shows the levels of 20 and 17 in the plasma over the course of the study. As can be seen, 20 was not seen in the plasma after oral dosing, whilst 17 was detected up to 8 hours after dosing. After iv administration, 20 was only detected up to 0.25 hours after dosing, whilst 17 was detected up to 4 hours after dosing. The data would suggest that 20 rapidly converts in vivo to 17 after either oral or iv dosing.

All references including patent and patent applications referred to in this application are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

### REFERENCES

Allen, I. W. and Ritchie, D. A. (1994) Cloning and analysis of DNA sequences from Streptomyces hygroscopicus encoding geldanamycin biosynthesis. Mol. Gen. Genet. 243: 593-599.
Bagatell, R. and Whitesell, L. (2004) Altered Hsp90 function in cancer: A unique therapeutic opportunity. Molecular Cancer Therapeutics 3: 1021-1030.
Beliakoff, J. and Whitesell, L. (2004) Hsp90: an emerging target for breast cancer therapy. Anti-Cancer Drugs 15:651-662.
Blagosklonny, M. V. (2002) Hsp-90-associated oncoproteins: multiple targets of geldanamycin and its analogues. Leukemia 16:455-462.
Blagosklonny, M. V., Toretsky, J., Bohen, S, and Neckers, L. (1996) Mutant conformation of p53 translated in vitro or in vivo requires functional HSP90. Proc. Natl. Acad. Sci. USA 93:8379-8383.
Bohen, S. P. (1998) Genetic and biochemical analysis of p23 and ansamycin antibiotics in the function of Hsp90-dependent signaling proteins. Mol Cell Biol 18:3330-3339.
Carreras, C. W., Schirmer, A., Zhong, Z. and Santi D. V. (2003) Filter binding assay for the geldanamycin-heat shock protein 90 interaction. Analytical Biochemistry 317:40-46.
Chiosis, G., Huezo, H., Rosen, N., Mimnaugh, E., Whitesell, J. and Neckers, L. (2003) 17AAG: Low target binding affinity and potent cell activity—finding an explanation. Molecular Cancer Therapeutics 2:123-129.
Chiosis, G., Vilenchik, M., Kim, J. and Solit, D. (2004) Hsp90: the vulnerable chaperone. Drug Discovery Today 9:881-888.
Csermely, P. and Soti, C. (2003) Inhibition of Hsp90 as a special way to inhibit protein kinases. Cell. Mol. Biol. Lett. 8:514-515.
DeBoer, C. and Dietz, A. (1976) The description and antibiotic production of Streptomyces hygroscopicus var. geldanus. J. Antibiot. 29:1182-1188.
DeBoer, C., Meulman, P. A., Wnuk, R. J., and Peterson, D. H. (1970) Geldanamycin, a new antibiotic. J. Antibiot. 23:442-447.
Dengler W. A., Schulte J., Berger D. P., Mertelsmann R. and Fiebig H H. (1995) Development of a propidium iodide fluorescence assay for proliferation and cytotoxicity assay. Anti-Cancer Drugs, 6:522-532.
Donzé O. and Picard, D. (1999) Hsp90 binds and regulates the ligand-inducible α subunit of eukaryotic translation initiation factor kinase Gcn2. Mol Cell Biol 19:8422-8432.
Egorin M J, Lagattuta T F, Hamburger D R, Covey J M, White K D, Musser S M, Eiseman J L. (2002) “Pharmacokinetics, tissue distribution, and metabolism of 17-(dimethylaminoethylamino)-17-demethoxygeldanamycin (NSC 707545) in CD2F1 mice and Fischer 344 rats.” Cancer Chemother Pharmacol, 49(1), pp 7-19.
Eustace, B. K., Sakurai, T., Stewart, J. K., et al. (2004) Functional proteomic screens reveal an essential extracellular role for hsp90α in cancer cell invasiveness. Nature Cell Biology 6:507-514.
Fang, Y., Fliss, A. E., Rao, J. and Caplan A. J. (1998) SBA1 encodes a yeast Hsp90 cochaperone that is homologous to vertebrate p23 proteins. Mol Cell Biol 18:3727-3734.
Fiebig H. H., Dengler W. A. and Roth T. Human tumor xenografts: Predictivity, characterization, and discovery of new anticancer agents. In: Fiebig H H, Burger A M (eds). Relevance of Tumor Models for Anticancer Drug Development. Contrib. Oncol. 1999, 54: 29-50.
Furniss B. S., Hannaford A. J., Smith P. W. G. and Tatchell A. R. (1989) Vogel's textbook of practical organic chemistry, 5^{th} Ed, Pearson, Prentice Hall, Harlow, UK
Goetz, M. P., Toft, D. O., Ames, M. M. and Ehrlich, C. (2003) The Hsp90 chaperone complex as a novel target for cancer therapy. Annals of Oncology 14:1169-1176.
Grass, G. M., Rubas, W., Jezyk, N., (1992) Evaluation of CACO-2 monolayers as a predictor of drug permeability in colonic tissues. FASEB Journal, 6, A1002.
Harris, S. F., Shiau A. K. and Agard D. A. (2004) The crystal structure of the carboxy-terminal dimerization domain of htpG, the Escherichia coli Hsp90, reveals a potential substrate binging site. Structure 12: 1087-1097.
Hong, Y.-S., Lee, D., Kim, W., Jeong, J.-K. et al. (2004) Inactivation of the carbamoyltransferase gene refines post-polyketide synthase modification steps in the biosynthesis of the antitumor agent geldanamycin. J. Am. Chem. Soc. 126:11142-11143.
Hostein, I., Robertson, D., DiStefano, F., Workman, P. and Clarke, P. A. (2001) Inhibition of signal transduction by the Hsp90 inhibitor 17-allylamino-17-demethoxygeldanamycin results in cytostasis and apoptosis. Cancer Research 61:4003-4009.
Hu, Z., Liu, Y., Tian, Z.-Q., Ma, W., Starks, C. M. et al. (2004) Isolation and characterization of novel geldanamycin analogues. J. Antibiot. 57:421-428.
Hur, E., Kim, H.-H., Choi, S. M., et al. (2002) Reduction of hypoxia-induced transcription through the repression of hypoxia-inducible factor-1α/aryl hydrocarbon receptor nuclear translocator DNA binding by the 90-kDa heat-shock protein inhibitor radicicol. Molecular Pharmacology 62:975-982.
Iwai Y, Nakagawa, A., Sadakane, N., Omura, S., Oiwa, H., Matsumoto, S., Takahashi, M., Ikai, T., Ochiai, Y. (1980) Herbimycin B, a new benzoquinoid ansamycin with anti-TMV and herbicidal activities. The Journal of Antibiotics, 33(10), pp 1114-1119.
Jez, J. M., Chen, J. C.-H., Rastelli, G., Stroud, R. M. and Santi, D. V. (2003) Crystal structure and molecular modeling of 17-DMAG in complex with human Hsp90. Chemistry and Biology 10:361-368.
Kaur, G., Belotti, D, Burger, A. M., Fisher-Nielson, K., Borsotti, P. et al. (2004) Antiangiogenic properties of 17-(Dimethylaminoethylamino)-17-Demethoxygeldanamycin: an orally bioavailable heat shock protein 90 modulator. Clinical Cancer Research 10:4813-4821.
Kumar, R., Musiyenko, A. and Barik S. (2003) The heat shock protein 90 of Plasmodium falciparum and antimalarial activity of its inhibitor, geldanamycin. J Malar 2:30.
Kurebayashi, J., Otsuke, T., Kurosumi, M., Soga, S., Akinaga, S, and Sonoo, H. (2001) A radicicol derivative, KF58333, inhibits expression of hypoxia-inducible factor-1α and vascular endothelial growth factor, angiogenesis and growth of human breast cancer xenografts. Jpn. J. Cancer Res. 92:1342-1351.
Le Brazidec, J.-Y., Kamal, A., Busch, D., Thao, L., Zhang, L. et al. (2003) Synthesis and biological evaluation of a new class of geldanamycin derivatives as potent inhibitors of Hsp90. J. Med. Chem. 47: 3865-3873.
Lee, Y.-S., Marcu, M. G. and Neckers, L. (2004) Quantum chemical calculations and mutational analysis suggest heat shock protein 90 catalyzes trans-cis isomeration of geldanamycin. Chem. Biol. 11:991-998.
Li, A. P. (1992) Screening for human ADME/Tox drug properties in drug discovery. Drug Discovery Today, 6, 357-366
Liu, X.-D., Morano, K. A. and Thiele D. J. (1999); The yeast Hsp110 family member, Sse1, is an Hsp90 cochaperone. J Biol Chem 274:26654-26660.
Mander, R., Wu, C., Sausville, E. A., Roettinger, A. J., Newman, D. J., Ho, D. K., King, R., Yang, D., Lippman, M. E., Landolfi, N. F., Dadachova, E., Brechbiel, M. W. and Waldman, T. A. (2000) Immunoconjugates of geldanamycin and anti-HER2 monoclonal antibodies: antiproliferative activity on human breast carcinoma cell lines. Journal of the National Cancer Institute 92:1573-1581.
McLaughlin S. H., Smith, H. W. and Jackson S. E. (2002) Stimulation of the weak ATPase activity of human Hsp90 by a client protein. J. Mol. Biol. 315: 787-798.
Muroi, M., Izawa M., Kosai, Y., and Asai, M. (1980) Macbecins I and II, New Antitumor antibiotics. II. Isolation and characterization. J Antibiotics 33:205-212.
Muroi M, Izawa M, Kosai Y, Asai M. (1981) “The structures of macbecin I and II” Tetrahedron, 37, pp 1123-1130.
Muroi, M., Izawa M., Kosai, Y., and Asai, M. (1980) Macbecins I and II, New Antitumor antibiotics. II. Isolation and characterization. J Antibiotics 33:205-212.
Neckers, L (2003) Development of small molecule Hsp90 inhibitors: utilizing both forward and reverse chemical genomics for drug identification. Current Medicinal Chemistry 9:733-739.
Neckers, L. (2002) Hsp90 inhibitors as novel cancer chemotherapeutic agents. Trends in Molecular Medicine 8:S55-S61.
Nimmanapalli, R., O'Bryan, E., Kuhn, D., Yamaguchi, H., Wang, H.-G. and Bhalla, K. N. (2003) Regulation of 17-AAG-induced apoptosis: role of Bcl-2, Bcl-x_{L}, and Bax downstream of 17-AAG-mediated down-regulation of Akt, Raf-1, and Src kinases. Neoplasia 102:269-275.
Omura, S., Iwai, Y., Takahashi, Y., Sadakane, N., Nakagawa, A., Oiwa, H., Hasegawa, Y., Ikai, T., (1979), Herbimycin, a new antibiotic produced by a strain of Streptomyces. The Journal of Antibiotics, 32(4), pp 255-261.
Omura, S., Miyano, K., Nakagawa, A., Sano, H., Komiyama, K., Umezawa, I., Shibata, K, Satsumabayashi, S., (1984), “Chemical modification and antitumor activity of Herbimycin A. 8,9-epoxide, 7,9-carbamate, and 17 or 19-amino derivatives”. The Journal of Antibiotics, 37(10), pp 1264-1267.
Ono, Y., Kozai, Y. and Ootsu, K. (1982) Antitumor and cytocidal activities of a newly isolated benzenoid ansamycin, Macbecin I. Gann. 73:938-44.
Patel, K., M. Piagentini, Rascher, A., Tian, Z. Q., Buchanan, G. O., Regentin, R., Hu, Z., Hutchinson, C. R. And McDaniel, R. (2004). “Engineered biosynthesis of geldanamycin analogs for hsp90 inhibition.” Chem Biol 11(12): 1625-33.
Rascher, A., Hu, Z., Viswanathan, N., Schirmer, A. et al. (2003) Cloning and characterization of a gene cluster for geldanamycin production in Streptomyces hygroscopicus NRRL 3602. FEMS Microbiology Letters 218:223-230.
Rascher, A., Z. Hu, Buchanan, G. O., Reid, R. and Hutchinson, C. R. (2005). Insights into the biosynthesis of the benzoquinone ansamycins geldanamycin and herbimycin, obtained by gene sequencing and disruption. Appl Environ Microbiol 71(8): 4862-71.
Roth T., Burger A. M., Dengler W., Willmann H. and Fiebig H. H. Human tumor cell lines demonstrating the characteristics of patient tumors as useful models for anticancer drug screening. In: Fiebig H H, Burger A M (eds). Relevance of Tumor Models for Anticancer Drug Development. Contrib. Oncol. 1999, 54: 145-156.
Rowlands, M. G., Newbatt, Y. M., Prodromou, C., Pearl, L. H., Workman, P. and Aherne, W. (2004) High-throughput screening assay for inhibitors of heat-shock protein 90 ATPase activity. Analytical Biochemistry 327:176-183
Schulte, T. W., Akinaga, S., Murakata, T., Agatsuma, T. et al. (1999) Interaction of radicicol with members of the heat shock protein 90 family of molecular chaperones. Molecular Endocrinology 13:1435-1488.
Shibata, K., Satsumabayashi, S., Nakagawa, A., Omura, S. (1986a) The structure and cytocidal activity of herbimycin C. The Journal of Antibiotics, 39(11), pp 1630-1633.
Shibata, K., Satsumabayashi, S., Sano, H., Komiyama, K., Nakagawa, A., Omura, S. (1986b) Chemical modification of Herbimycin A: synthesis and in vivo antitumor activities of halogenated and other related derivatives of herbimycin A. The Journal of Antibiotics, 39(3), pp 415-423.
Schnur, R. C., Corman, M. L., Gallaschun, R. J., Cooper, B. A., Dee, M. F., Doty, J. L., Muzzi, M. L., Moyer, J. D., DiOrio, C. I. et al. (1995). Inhibition of the oncogene product p185erbB-2 in vitro and in vivo by geldanamycin and dihydrogeldanamycin derivatives. Journal of Medicinal Chemistry, 38(19), pp 3806-12.
Smith M. B. and March J. (2001) March's advanced organic chemistry, 5^{th} Ed, John Wiley and Sons Inc., UK
Smith-Jones, P. M., Solit, D. B., Akhurst, T., Afroze, F., Rosen, N. and Larson, S. M. (2004) Imaging the pharmacodynamics of HER2 degradation in response to Hsp90 inhibitors. Nature Biotechnology 22:701-706.
Sreedhar A. S., Nardai, G. and Csermely, P. (2004) Enhancement of complement-induced cell lysis: a novel mechanism for the anticancer effects of Hsp90 inhibitors. Immunology letters 92:157-161.
Sreedhar, A. S., Söti, C. and Csermely, P. (2004a) Inhibition of Hsp90: a new strategy for inhibiting protein kinases. Biochimica Biophysica Acta 1697:233-242.
Stead, P., Latif, S., Blackaby, A. P. et al. (2000) Discovery of novel ansamycins possessing potent inhibitory activity in a cell-based oncostatin M signalling assay. J Antibiotics 53:657-663.
Supko, J. G., Hickman, R. L., Greyer, M. R. and Malspeis, L (1995) Preclinical pharmacologic evaluation of geldanamycin as an antitumor agent. Cancer Chemother. Pharmacol. 36:305-315.
Takahashi, A., Casais, C., Ichimura K. and Shirasu, K. (2003) HSP90 interacts with RAR1 and SGT1 and is essential for RPS2-mediated disease resistance in Arabidopsis. Proc. Natl. Acad. Sci. USA 20:11777-11782.
Tanida, S., Hasegawa, T. and Higashide E. (1980) Macbecins I and II, New Antitumor antibiotics. I. Producing organism, fermentation and antimicrobial activities. J Antibiotics 33:199-204.
Tian, Z.-Q., Liu, Y., Zhang, D., Wang, Z. et al. (2004) Synthesis and biological activities of novel 17-aminogeldanamycin derivatives. Bioorganic and Medicinal Chemistry 12:5317-5329.
Uehara, Y. (2003) Natural product origins of Hsp90 inhibitors. Current Cancer Drug Targets 3:325-330.
Vasilevskaya, I. A., Rakitina, T. V. and O'Dwyer, P. J. (2003) Geldanamycin and its 17-Allylamino-17-Demethoxy analogue antagonize the action of cisplatin in human colon adenocarcinoma cells: differential caspase activation as a basis of interaction. Cancer Research 63: 3241-3246.
Volpe, D. A., Faustino, P. J., Yu, L. X., (2001) Towards standardisation of an in vitro method of drug absorption. Pharmacopeial Forum, 27, 2916-2922
Watanabe, K., Okuda, T., Yokose, K., Furumai, T. and Maruyama, H. H. (1982) Actinosynnema mirum, a new producer of nocardicin antibiotics. J. Antibiot. 3:321-324.
Wegele, H., Müller, L. and Buchner, J. (2004) Hsp70 and Hsp90-a relay team for protein folding. Rev Physiol Biochem Pharmacol 151:1-44.
Whitesell, L., Mimnaugh, E. G., De Costa, B., Myers, C. E. and Neckers, L. M. (1994) Inhibition of heat shock protein HSP90-pp 60^{v-src} heteroprotein complex formation by benzoquinone ansamycins: Essential role for stress proteins in oncogenic transformation. Proc. Natl. Acad. Sci. USA 91: 8324-8328.
Winklhofer, K. F., Heller, U., Reintjes, A. and Tatzelt J. (2003) Inhibition of complex glycosylation increases the formation of PrP^{sc}. Traffic 4:313-322.
Workman P. (2003) Auditing the pharmacological accounts for Hsp90 molecular chaperone inhibitors: unfolding the relationship between pharmacokinetics and pharmacodynamics. Molecular Cancer Therapeutics 2:131-138.
Workman, P. and Kaye, S. B. (2002) Translating basic cancer research into new cancer therapeutics. Trends in Molecular Medicine 8:S1-S9.
Young, J. C.; Moarefi, I. and Hartl, U. (2001) Hsp90: a specialized but essential protein folding tool. J. Cell. Biol. 154:267-273.

1. A derivative of a C21-deoxy ansamycin, or a salt thereof, which contains a 1-hydroxyphenyl moiety bearing at position 3 an aminocarboxy substituent, in which position 5 and the aminocarboxy substituent at position 3 are connected by an aliphatic chain of varying length characterised in that the 1-hydroxy position of the phenyl ring is derivatised by an aminoalkyleneaminocarbonyl group, which alkylene group, which may optionally be substituted by alkyl groups, has a chain length of 2 or 3 carbon atoms or a phosphoric acid, or a phosphoric acid ester group, and which derivatising group increases the water solubility and/or the bioavailability of the parent molecule.

2. A derivative of a C21-deoxy ansamycin, or a salt thereof, according to claim 1, which contains a 1-hydroxyphenyl moiety bearing at position 3 an aminocarboxy substituent, in which position 5 and the aminocarboxy substituent at position 3 are connected by an aliphatic chain of varying length characterised in that the 1-hydroxy position of the phenyl ring is derivatised by a phosphoric acid or a phosphoric acid ester group, and which derivatising group increases the water solubility and/or the bioavailability of the parent molecule.

3. A derivative of a C21-deoxy ansamycin, or a salt thereof, according to claim 1, which contains a 1-hydroxyphenyl moiety bearing at position 3 an aminocarboxy substituent, in which position 5 and the aminocarboxy substituent at position 3 are connected by an aliphatic chain of varying length characterised in that the 1-hydroxy position of the phenyl ring is derivatised by an aminoalkyleneaminocarbonyl group, which alkylene group, which may optionally be substituted by alkyl groups, has a chain length of 2 or 3 carbon atoms or a phosphoric acid, or a phosphoric acid ester group, and which derivatising group increases the water solubility and/or the bioavailability of the parent molecule and which is capable of being removed in vivo.

4. A C21-deoxy ansamycin derivative according to the formulas (IA-IC) below, or a pharmaceutically acceptable salt thereof: 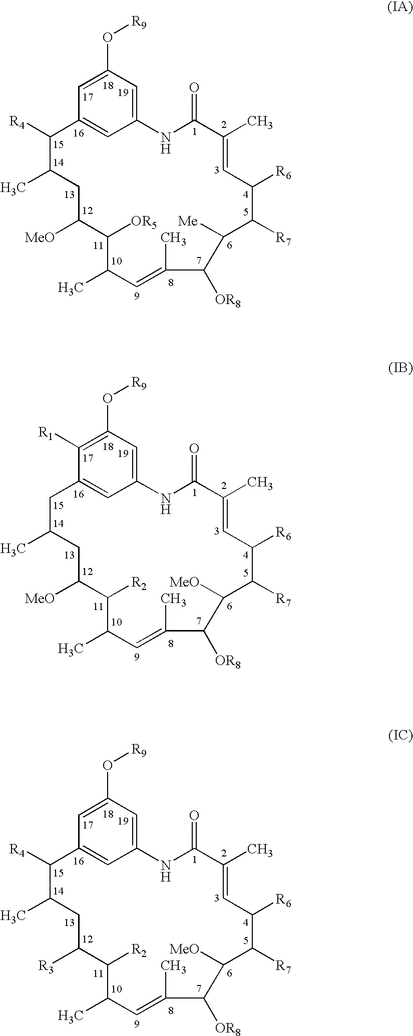
wherein:
R₁ represents H, OH, OMe;
R₂ represents OH, OMe or keto;
R₃ represents OH or OMe;
R₄ represents H, OH or OCH₃;
R₅ represents H or CH₃;
R₆ and R₇ either both represent H or together they represent a bond (i.e. C4 to C5 is a double bond);
R₈ represents H or —C(O)—NH₂;
R₉ represents, 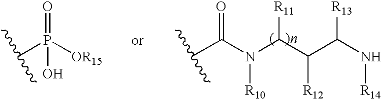
wherein:
n represents 0 or 1;
R₁₀ represents H, Me, Et or iso-propyl;
R₁₁, R₁₂ and R₁₃ each independently represent H or a C1-C4 branched or linear chain alkyl group; or R₁₁ and R₁₂, or R₁₂ and R₁₃, may be connected so as to form a 6-membered carbocyclic ring;
R₁₄ represents H or a C1-C4 branched or linear chain alkyl group; and
R₁₅ represents H, Me or Et.

5. A compound according to claim 4 wherein:
R₁ represents H, OH, OMe;
R₂ represents OH, OMe or keto;
R₃ represents OH or OMe;
R₄ represents H, OH or OCH₃;
R₅ represents H or CH₃
R₆ and R₇ either both represent H or together they represent a bond (i.e. C4 to C5 is a double bond);
R₈ represents H or —C(O)—NH₂;
R₉ represents 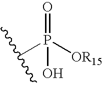
wherein:
R₁₅ represents H, Me or Et.

6. A compound according to claim 4 wherein:
R₁ represents H, OH, OMe;
R₂ represents OH, OMe or keto;
R₃ represents OH or OMe;
R₄ represents H, OH or OCH₃;
R₅ represents H or CH₃
R₆ and R₇ either both represent H or together they represent a bond (i.e. C4 to C5 is a double bond);
R₈ represents H or —C(O)—NH₂;
R₉ represents 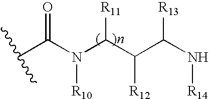
wherein:
n represents 0 or 1;
R₁₀ represents H, Me, Et or iso-propyl;
R₁₁, R₁₂ and R₁₃ each independently represent H or a C1-C4 branched or linear chain alkyl group; or R₁₁ and R₁₂, or R₁₂ and R₁₃, may be connected so as to form a 6-membered carbocyclic ring; and
R₁₄ represents H or a C1-C4 branched or linear chain alkyl group.

7. A compound according to claim 4 wherein R₁ represents H,

8. A compound according to claim 4 wherein R₁ represents OMe.

9. A compound according to claim 4 wherein R₂ represents OH.

10. A compound according to claim 4 wherein R₂ represents OMe.

11. A compound according to claim 4 wherein R₃ represents OMe.

12. A compound according to claim 4 wherein R₄ represents H.

13. A compound according to claim 4 wherein R₄ represents H.

14. A compound according to claim 4 wherein R₅ represents H.

15. A compound according to claim 4 wherein R₆ represents H.

16. A compound according to claim 4 wherein R₇ represents H.

17. A compound according to claim 4 wherein R₈ represents —C(O)—NH₂.

18. A compound according to claim 4, wherein R₁₅ represents H.

19. A compound according to claim 4, wherein R₁₅ represents Me or Et.

20. A compound according to claim 4 wherein R₁₀ represents Me.

21. A compound according to claim 4 wherein R₁₀ represents Et.

22. A compound according to claim 4, wherein R₁₄ represents Me.

23. A compound according to claim 4, wherein R₁₄ represents Et.

24. A compound according to claim 4, wherein R₁₁ represents H.

25. A compound according to claim 4, wherein R₁₂ represents H.

26. A compound according to claim 4, wherein R₁₃ represents H.

27. A compound according to claim 4, wherein n represents 0.

28. A compound according to claim 4 wherein n is 0, R₁₂ and R₁₃ each represent H and R₁₀ and R₁₄ each represent Me.

29. A compound according to claim 4 wherein n is 0, R₁₂ and R₁₃ each represent H and R₁₀ and R₁₄ each represent Et.

30. A compound according to claim 4 which is a compound of structure (IA).

31. A compound according to claim 4 which is a compound of structure (IB).

32. A compound according to claim 4 which is a compound of structure (IC).

33. A compound according to claim 4 which is: 4,5-dihydro-11-O-demethyl-15-demethoxy-18,21-didehydro-21-deoxomacbecin-18-phosphate or a pharmaceutically acceptable salt thereof.

34. A compound according to claim 4 which is of formula: 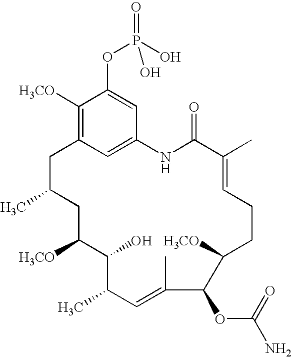
or a pharmaceutically acceptable salt thereof.

35. A compound according to 4, in the form of a monosodium salt.

36. A compound according to claim 4 which is 18-O-(N,N′-dimethylpropanediamine carbamoyl)-4,5-dihydro-11-O-demethyl-15-demethoxy-18,21-didehydro-18-hydroxy-21-deoxomacbecin or a pharmaceutically acceptable salt thereof.

37. A compound according to claim 4 which is 18-O-(N,N′-diethylethylenediamine carbamoyl)-4,5-dihydro-11-O-demethyl-15-demethoxy-18,21-didehydro-18-hydroxy-21-deoxomacbecin or a pharmaceutically acceptable salt thereof.

38. A compound according to claim 4 which is 18-O-(N,N′-dimethylethylenediamine carbamoyl)-4,5-dihydro-11-O-demethyl-15-demethoxy-18,21-didehydro-18-hydroxy-21-deoxomacbecin or a pharmaceutically acceptable salt thereof.

39. A compound according to claim 4 which is of formula: 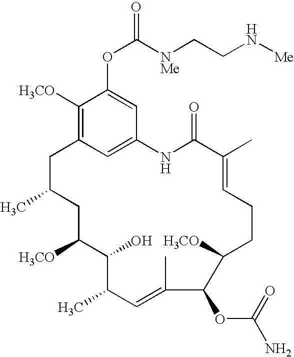
or a pharmaceutically acceptable salt thereof.

40. A compound according to claim 4 which is of formula: 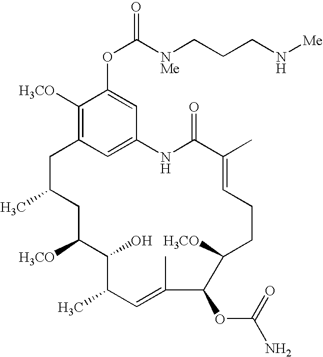
or a pharmaceutically acceptable salt thereof.

41. A compound according to claim 4 which is of formula: 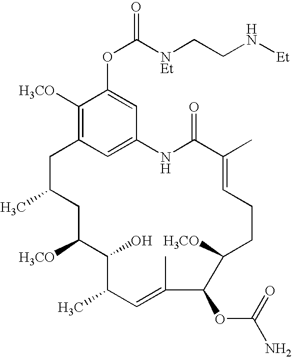
or a pharmaceutically acceptable salt thereof.

42. A pharmaceutical composition comprising an C21-deoxy ansamycin derivative or a pharmaceutically acceptable salt thereof according to claim 1, together with one or more pharmaceutically acceptable diluents or carriers.

43-45. (canceled)

46. A method of treatment of cancer, B-cell malignancies, malaria, fungal infection, diseases of the central nervous system and neurodegenerative diseases, diseases dependent on angiogenesis, autoimmune diseases or a prophylactic pretreatment for cancer, which comprises administering to a patient in need thereof an effective amount of a C21-deoxy ansamycin derivative or a pharmaceutically acceptable salt thereof according to claim 1.

47. A process for preparing a C21-deoxy ansamycin derivative or a pharmaceutically acceptable salt thereof according to claim 4 which comprises
(a) reacting a compound of formula (IIA), (IIB) or (IIC): 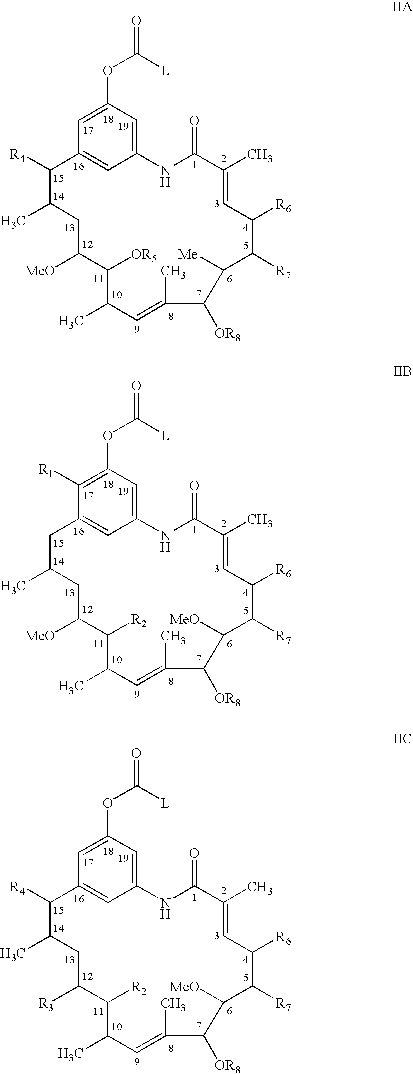
wherein L is a leaving group
or a protected derivative thereof, with a compound of formula (V) 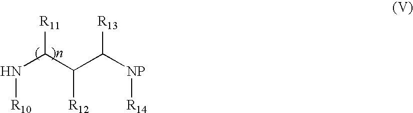
wherein P represents a protecting group
and wherein n and R₁-R₈ and R₁₀ to R₁₄ are as described in claim 4; or
(b) reacting a compound of formula (IIIA), (IIIB) or (IIIC) 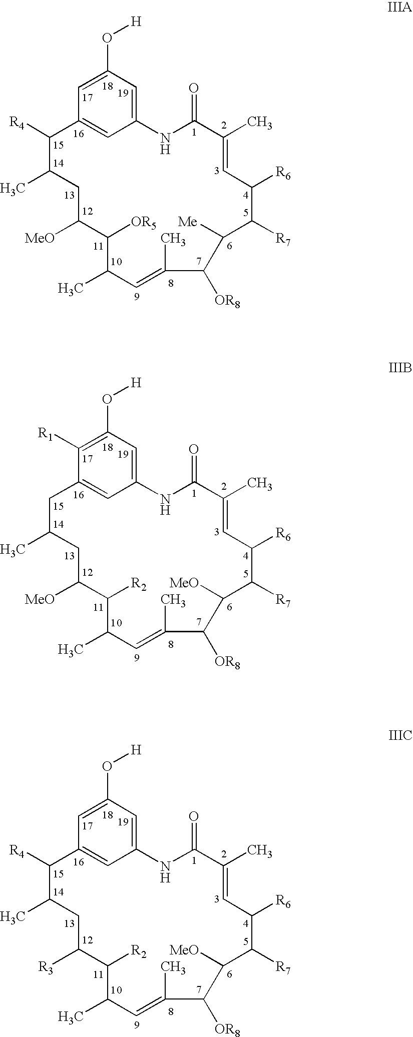
or a protected derivative thereof
and wherein R₁-R₈ are as described in claim 4, with a phosphorylating reagent; or
(c) converting a compound of formula (I) or a salt thereof to another compound of formula (I) or another pharmaceutically acceptable salt thereof; or
(d) deprotecting a protected compound of formula (I).

48. A compound of formula (IIA), (IIB) or (IIC), or a salt thereof: 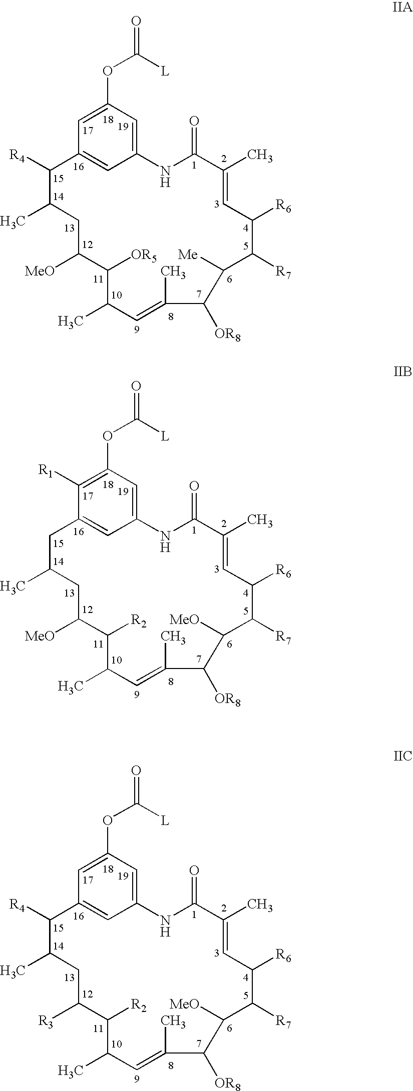
wherein R₁-R₈ are as defined in claim 4 and L is a leaving group.

49. A pharmaceutical composition comprising an C21-deoxy ansamycin derivative or a pharmaceutically acceptable salt thereof according to claim 4, together with one or more pharmaceutically acceptable diluents or carriers.

50. A method of treatment of cancer, B-cell malignancies, malaria, fungal infection, diseases of the central nervous system and neurodegenerative diseases, diseases dependent on angiogenesis, autoimmune diseases or a prophylactic pretreatment for cancer, which comprises administering to a patient in need thereof an effective amount of a C21-deoxy ansamycin derivative or a pharmaceutically acceptable salt thereof according to claim 4.


SEQUENCE LISTING (21 sequences)

SEQ ID NO: 1 - DNA, 33 bp, Artificial
  Primer
ggtctagaggtcagtgcccccgcgtaccgtcgt

SEQ ID NO: 2 - DNA, 26 bp, Artificial
  Primer
ggcatatgcttgtgctcgggctcaac

SEQ ID NO: 3 - DNA, 36 bp, Artificial
  primer
cccgcccgcgcgagcggcgcgtggccgcccgagggc

SEQ ID NO: 4 - DNA, 36 bp, Artificial
  Primer
gcgtcctcgcgcagccacgccaccagcagctccagc

SEQ ID NO: 5 - DNA, 35 bp, Artificial
  Primer
ccaaccccgccgcgtccccggccgcgccgaacacg

SEQ ID NO: 6 - DNA, 34 bp, Artificial
  Primer
gtcgtcggctacgggccggtggggcagctgctgt

SEQ ID NO: 7 - DNA, 35 bp, Artificial
  Primer
gtcggtggactgccctgcgcctgatcgccctgcgc

SEQ ID NO: 8 - DNA, 35 bp, Artificial
  Primer
ggccggtggtgctgcccgaggacggggagctgcgg

SEQ ID NO: 9 - DNA, 39 bp, Artificial
  Primer
caccgctcgcgggggtggcgcggcgcacgacgtggctgc

SEQ ID NO: 10 - DNA, 38 bp, Artificial
  Primer
cctcctcggacagcgcgatcagcgccgcgcacagcgag

SEQ ID NO: 11 - DNA, 100588 bp, Actinosynnema pretiosum
gatctggggcgacgagccgcccgccgggccggggccggcgttgcaggcgctcgtctcccggctgcggcgggcgctcggcgcgccgggcgcggtcgcgctgggggtgggcgggtaccggctcgtggcggacgtggacgcggcgcggttcgaggagctggccgcgcggggcggggaggacgcgctgcgggaggccgccgcgctgtggggcgggcgggtcgggggcgagccgccggtggtcgcggccgtcgcgccgcgggtggcgacccggctggcgcggctgtcggtggaggtggtgctggacctggcggaggtcgagctggcgctcgggcgcaccggggcggccatcggtggggcgagcggggtgctggccgagcacccggcgcacgagcgggccgccggggtgctggtggacgcgctcgcgggcgcgggacggcaggccgaggcgctggcggcctacgagcgggtccgcgcggcgctggccgacgagctgggcgccgaccccggcacggccctgcgcgagcgccacctgcggctgctgcgcgccaccccgccaccgctcccccggccgaacgcgctgcccgcgccggtgacgggcttcctcggccgggacgccgacctcgcccgcgtcgccgacctgctggccgccgggcggctggtcaccgtcgtcgggcccggcggggtgggcaagacccggctggccgtggaggcgctgcgccgggaccgggacgcgctgctggtggacctcgcgccggtcgccgagccctcggaggtcgtcgccgccgtgctcgccgggatcgggctgcgcggcgaccgcgaccggccgggcggggacgcgacggcgctgctggccgccgagctggcggcgcgcaggtcggtgctgctgctggacaactgcgagcacctggtcgacgccgtggcccacctggtcgcgctcctgctcccccgctgccccgagctgcgcgtgctcgccaccagccgggaacccctggcggtcgacggggaggcgctggtcccgctggggccgctcgcgctgcccggaatcggggacgggcttgacgccgcggtcggcacggcctcggtgcggttgttcgcccaacgggcgtcggcggtgcgccccggtttcgccgtcgacgccacgacgctgccggacgtggtgcgcctggtgcgggcgctggacgggctgccgctggcgctggagctggccgccgcccggttgcgcgccctgccgctgcccgacctggtggccgggttgtcggcgcggttccgcctgctggcgggcgggaaccgggccgcgccgccccggcaccgcacgctgcgcgcggtgatcgcgtggagctgggacctgctggacgggcccgagcgggccgtggccgagcggatctccgtgctgcccggcggggtcaccccggagtcggccgccgccgtctgcgcgggcgccgtgcccgccgacgaggtgcccgaactgctggccgcgctggtcgaccggtcgctgctgagcctggtcgggggtcggcggcggatgctggagacggtgcgcgcgtacggggtcgagcgcctggccgccgccggggacttgagcgcggtccgcgacctggccgccgcgcacgtggcgggggtgctggcggggcaggacgcggtgctgcgcgggccggggcagcgcgcggcggtggcggcgatcggcgcggagcacgacaacgcggtggccgcgctgcaccaccggtgcgccaccggggacgcggacggggcgctcgcgctggcgctgtcgctggtctggtactggcaggtgttcggccgccagtccgagggcgcgcactggctcgggcgggcgctggcggtgcccggcgggccgtccccggagcgggactgcgcgcgggccgcccacctgctcggcctggccgacggcgggcacggggtgggtgatcgcggggaggtgggggcgctcgcggaccgggtgctggcgcaccgggggctccccggtcacctgcgggtgctcggggcggtcctgctgttcctgctggggcgcggcgagggggtgttccgggagctgggcgcgggcggcgggtggttgtccgggctggcgcacctgttcctggccgagctggcggagaacgcgggcgagctggaccgggcgcgcgggcacgcggaggtgtccctggaccggttccgggcggccggggacgggtggggcgtggcgggggtgctgccggtgcgggcgcgggcgcggcggtacgacgacctggacgggacgtgggcggaccttcgggaggcgcgggcgctggagggggagttcggggcgctgagccccggtgaccgggtgcgggcggacctgcggtgggtcgacctgcacgagcggcgcggtgacagcggggcggcgctggaggtgctggccgcggcccgtgctcggggggagcaggtcgcggtggtggacgcgcgggaggccgcgctgcgggtgcggctcggggacctggggcgggcgggtgagctgctggccggggtgggtggggcggtgggcgacctggcgcgggccgcgtatcgggtggcctcgggggacctggcgggtgcggagcgggcgttgcggcgggcgcgggtggtggcggctgcgagcggggagctgcccgcgctggccccggtggcggtgggggcggcggcgctggagcaggcgcgggggcggtgggcggggtcgggggtgctgctcgggacggccgcgcgggtgcggggcgcgcacgaccgcaccgaccccctggtgcgcgagctggtcgaccgggggcgggcggcggtgggcgggagcgcgttcgcggcggcgtacgcgcgggggtgggaggcggagcgggacgtggcggcggcgttcgtgctctgagcgccgggatcgggcgggcggggtcaggcgggcggggtcatgtgggcggggtcaggcgggccaggtcacacgtccagggaccccgcccagtccgcgatcgtccggacttcggcctgcgtcgggaagaccttctcggtgagcacgcggtgcacctcggggtcgccgtccaggcagccgtcggccaggacggtgagctggaagtccaggtcggcggcctggcggagggtggacaggaccacgccgctggtcgcgatgccggtgagcaccaggtggtcgacgccctgggcgcgcaggacgaggtccaggtcgctgcccgcgaacgcgctgacgcggcgcttggtcaccaccacctcgtcgtcgagcggcgcggtctcggggtggaagtcggtggcgccggagcccctgggggccgcggccaggcggccgaacatcttgttgcgcgggtggatctccgcgtagtcggggcggaagccgacgccgacgtggatcaccggcacggacgcggcgcgggccgcctcgagcgcggtggcgagcctggggaggtaggccgggtcggggtagcgggcgaccacggcgggctggacgtccatcaccagcagggcgggggtggggatctcgggcctcgtttcggtggtggcggcgcgggggccgccggtgggggtcaggggtgcgggggtgccggggtgagcaggctggtgacggtgagcaggcggtcggcgagttcctcggggcgcagcgggtcctcggcgcgcacgacccagtcgtggacgatcgcgccggtgccgtgggagatcagcacggccaggtcgcgggcggcccgctcgtccacctcgcccaggccggcgcgcagggcctcggtggtgatgagggtgcggaagagctcggccagggcctccgccagccgccacgcgcacgggccggtgagcacggcgcggtagaaggggcggtggtcggcgaagtggcgggccacggccaggaggcgggcgtggcgcggggcccgcgggtcggccaggtgcggcaggagctcgcgccgcaccaggtccgccgcagcggcgacgaggagcgtgtcgcggtcgccgaagtgctggtagagcagctgcctgctgacgtcggcggcctcggccaggtcggtcaccgggaccgccgccccgcgctcggcgaccaggtcgacggcggcggccatgagggcggccctggagcgggcgacccggcggtcggggcgggtggtcacgggggtgaaactagacagttgtcaataaatgagcaagtgtcgtcgaacgcgcgcgcgggaatctccggtgcgcggggcccgtccctggcagcatgatcacgcgatgaccgaggtgaggacgcgcccgtacgccgggcccgcggacctgcgcgcgatgcaggggttggcgcggcggatctggacgccgtcgagccggtggcacgtcggcgacctggcctggcagcgcaaccagcacaccgggcgcgaggccgagtggccgaccgcgctgtgggaggcgggcggcgaggtggtggcgtgggggtgggccgagctgccgggtgagctggcgctgctggtcgaccccgcccggccggagcttgcgggggcggtgctcgactggttcgcgggcgtggccaccgcgccccggcggtcggtcaccgtgctggacgccgaaccgcacctggtcgccgcgctggaggctcgcgggtacgagcggctgggcgggccgcacttccggcactcggtgcgcgcgctggacgacctgccgacgcccgaactgcccgccgggtaccgggtccgcgccgtgcggggcgaggaggacgtggcggcgcgggtcgcggcgcaccgggcggcctggtggccgtcgcgggtcaccgaggagagctaccgggcggtgatgggggcgtggccgtaccggccggggctggactgggtggtggaggggccggacgggcggttcgcggccacctgcctgatctggttcgacgagcgcaacggcgtgggcgagctggaaccggtcggggtcgaccccggtctgcggcggcgcgggctggggcgggcggtgtgcctggcggcgctgggcgcgctgcgcgaggcgggcgggcgggcggcggtggtgtacccgctgcacgggcaccccgaccaccccgcgcccgcgccgctgtaccgggggctggggttccgcgagcacgcccgcacgatcaccttcaccgcgctggaggcgcgcgggtagcagcggccgggcggggcgagcggacccggtcgacgagcggctccgctgtcggagcggtcgtcccagcgcgtggacaccagtgccacgaccagaccgcgccccgcttcgcgtggtcggctcgggggtcgaccgcggtgaggctctcgccggggtgggtgaaccacgtcctggcgatggcctgcaccgcgagcaccgggtgccgcccgtggcgctggacgtcaccgacgcagccgccgtcgaccgggccgggccggccgcgttcggccgttgcgccgcgccggccgagtccgacgccaggtggcggccggtccccgggtccgcctggaactgaccccgccggcctccccgcccgcccgtccggccgggcgccgaacccgcctcaggcgtgctcgaccgcgcgcaccgatccccccaccaccaccggcatcgggacgtggtgcacggtcgtcgggctgcggtcgcggcgggggcgggacaggaggagttccacggccatcgcgcccaggcggtggtgcggcagggcgacggtggtcaggcgcgggcgcatccaggcggccacggggtggtcgtcgaagccgaccacggagacgtcgtccggcacggacaggcccgcctccgcgagcgcctggcacgcgccgaacgccaggcggtcgttgaagcacagcagcgcgcgagggcggtggtgggacaggaggtccagggtggcgcggtagccgttctccggcatccactccacgcacgggcgcacgctctccacctccacccccgccgccgcgaaggtctccagcgcgccggagaggcgggccacggcggcgatgtggcgcgggtcgatcgcctcggccgtgggcccggtgccgatcaggtgcacgccctcgcggtgcccggcgtcgagcagcacgcgcgccgccgaacggccgccgccgcggtcgtcggggagcacggcgtgcgcggggaagtcgttggcgggcagcacgttcagcagcacggacggcccgtcgcgcagcccgtccgggacctccagcagccgggggaacctggccgcgaagaccacgccctccacctggcgggcgcgcagcgaggccaccagcgccgcctccacctcgcggtcgccgccgctctcaccggcgaacagggtgaacccgtgccggtgggcggcgccgaccgcgccctcgatcagctcaccggacagcttggccgaggccacggcgtccgagacgaaaccgagggtcttggtgcgggaggcggacagcagcgtgtcgcggcggtagccgagctgctcggccgtcgcccgcaccttgcgctccaccgccgccgagatgcgcagctcccgagcgcggccggagagcaccagggaggcggtgggcaccgacacggcgcaggcggacgcgacgtcggccagcgtgacgcgcgtccgcccgctctcgcggacacctgctcgcgggggtgtgcccgtcacccgtgcctcccgtcaccggtcgcgcgacagccccgcgcgaggtcctaccccatcgtgcaggccgcgccgttcaaggagaaccccgaaggtggggccgcgtccccgccgtgggtgacctggtagccgatgctgactttgccaccgggtgggatcgccgcgttgtagcccgcgtcgcgggcggtcacccggcccgagctgggcgcgtacgaggcgttccagccggaggtgatcacctggcccgcgggcagcgcgaactccagcgaccagccctgcacctgcgtggtcccggtgttggtgatggcgagctccgccgtcaggccgttgccccaggcgttgacggtggccgacacccggcaggcccccggctgcggttcgccgggcgtggtggtggtggtggtggtggtggtcgtggtcgtggtggtgctggtcgggtcggggccggttccggcgaactgggtgaagaaccgccaggtctcctcgggcgcccacgtcctggtgccgctgtcgccgggcgcgttgtcctgcggtgcggcgatgtggccctcgtcgaacgcgacccagcgcaccgggtagccgtcgcggcagccggtgtaggtggtgccccggtgggtcaggctgccctgggacggttccggcgggttctgcgcggcgcagccgttgttgcgcacgaaccggtcgcgcatcgagcgcccgccggagatgttcaggacgctgtcgcgcaggccgtggatgccgaggtaggcgatgggctgcgtgccgccggcgcagccgctgagcacgccgcccgcgatgaccgcgaccgcgcggaacaccgtcggccgcgagcaggccaccgagtaggacatcgcgccgccgtagctgaagccggtggcgaaccgctgggtggtgtccacgcacagcccggcgtcgagctggcggacgatgtcgtcgacgagggtgatgtcctcgccgccgttgttggcccagccgttgttgaagccctgcggcgccacgaagatcgtgctgctgcccgccaggcgcttgaggccgtagtaggaccagacgtcccgctgcacggtctggccggtggcgacgtcgttcgcggtgccgctgagccagtggaagccgaagacgacgcggtgggggcggttccggtcgtagccgtccgggatcgacaggatgtaggtgcgggacttgccgctgctggtgatcgtgcgcgtgccgctggtgagcgcgggcgccttgccgcagccctccgtcgtggcggacgcgccgggggcgccggacgcgccgggcgctccggtcgcgctggtggtgatcagccccgcggcgagggtgagcagcgcgatgcccgctgccgcgaggaccctgttgcgcgccaagggattcgcccttcctgtggtggttccggtgggtgtggtcacggggtggtgaggtcgaagcggcgggcggtgacggagccgccgagcgcggcggtggcgtggttgaagacggcgaagcggtagcccatgaagaaccgccagtcgttcttgagcgtgaacgccgggccgaaggcggtgaagttgacgccgtcggtgctgtaggagaaccgggcctgcctgccgttgccggggcggatgtcggcgttggcgcgcaaccagatccgggagccgcccaggtcggcgctcgcgacctcgtagccggttccggtggtgcgccaggagccgtccatggtcaggccggtgacggagacgatccggttgcggccgttgtcgcgcttgacgccgatccacgccgaggagtcgcgcagcacggccagcccggtgcggtcgccgtcgcgcatccccgacaggtccagttccacggtgccggtggaggtggggccctggatgcggtgggtgagggtgttgcgggcggagtacaggtcgttggtgacggtcgcggtggacaggcgaaggccgttgttcacgctgtacttggcggtgtccgggttgtggttccactcccactgcgggccgagcgcggcgccggagaaggtgtcggcgccgatcatgggtttgacctggcgcgggggcgcgggcaggttcggcttcgggtaggtcgcgccccagccgccgttgacggtggtgacgcgcggccagccgtccgaggtccaggtgatcggggcgagcaccggcacgcgcccgccggggtaggcgtcgacgaacgccaggtagtgccagtcgccgttctgggtctgcaccaggccgccctggtgcggcactcccccgccctggatcggcgagggcaggtcgagcagcacctgctggatcgagtacgggccgaacgggctggacgacttgagcacgtactggccgttcgcgggcctggtgagccagatgtagtagttgccgccgcgcttgtagaagcgcgccccttcgagggtgccgatgttcgagggggtctggaacacctgctgggagcggacctccgacttcccgtcggcggagagctgggcgacgctgatgctggtgttgccgtaggcgacgtacagggtgtcgtcgtcgtccacgagcatcccggcgtcgtagtagcacttgttgatggtggtgtgcttggaccactggccgtcgacggcggtcgcggtgtacaggtgcgtctgggcgaagtcgacgcagccgccccagtagaaggtgcggttgctcttgcggtgcgccaggaacgacgcccagatgccgttgacgtacgcgcgggagccgttgcccatgtcgtacttggccccgaagtccaggcgtggcacggagtgcccggcgaactcccagttgaccaggtcgtaggagcgcagcacgggcgcgccgggcgagtagtgcatggtggaggccgagtagtagtaggtgtcgtccacgcgcaggacgtcgatgtcggcgaagtcctgccacagcacggggttggtgtaggtcccggccgcgcgggccgggtgggtggtggtggcggtcaggctcgccgccacgaggccgagcacggccagggcgatgggcgcccatcggcgttgacggggcatcggtgtgcctctcctggtgtccgggagttggctctgggcgcggcggcggtggacttgtcgggcgcggcggtggtcgtgggggtcagcagggggagttggtctgggtcagcaggcccagccgccagggcaggcggttgtagtcgccggacgcgttggggtccaggccctggtacaggtagctcagcttgcaggggttgatctccatggtctggtcggtcccgctgcgcaccagctcgccgtggctgatgtcgcgcgtccactggccaccggggaacgtggtgttgttggccctggcgaacgggttcgactcgctgtcggccagcgcggtccacggtccggcgatcgccggggcggtccaggagcggaaccagcggcggccgtccgagccgatcgcctcgtggagcatcagccactggttcttgccggcgaccttgtagatgttggacgcctcgaacaaccggttgcggttgctgtcctgcatggcgatcacggtgttggtgaagccgttggggaactgggcgaggctggtctccgagcggtacaggtggccgttgtcgtccgaggagaacaggtggcacttggccgtgtcgcagacggtccagaagtcgacccagtagccgttgccgatgttgtcccggatgatctgcggcatcccgttggcgtagaagttcctcggcgcggaccaggacgcggggttctcgatgtcggcggtcgtcgagtacgaggcgttggacccggtctggtacaccaggtaccacaggcgttgcggggcgaagtagaacacctgcggcgcggcccggtagcccgtgccgatcccggagcggtccaggtagtggtgcggggcggacgcggcctgggaccagtcggtgaagctggtgtgcacgaggttgtagccgttggtgtagaccgaggcgaacacgtggtagcggccgttgtggcgcaccacgctggggtccttgacggagaccgtggcgtgcgaggagtcgggcttgggtccgatcagcgcgccgctggaggaccaccggaagctgctcggcagcgagccgcccggttgctgcggggtcgtggtggtgggcggcgtggtcgggggcgtggtggtcgtgggcccgactcctcccgtgcacgtggtcccgttgaggctgaacgaggtcgggtcggggttggacccggtggaggtcgcggtgaacccgaactcgacgcggcccccggtggggatggcggcgttgtaggaggcgttgcgggcgctcacctggccgccggactgggacacctcggcgttccaggcctgcgcgacctgctggcccgagccgtaggtccaggtgagcgtccagccgtcgacggcgtcaccgaggttggtgatggcgacgctcgcggtgaaaccgccctgccactgggaggtcgccgcgtaggcgatcgagcaccccggcgccgcggcggcctggggtgggagggcggcgagcgcggccaccatggcgagcgaggtggtggccatggcgccgatccgggcccggcgcggggtgaacagccttgcgaggagcatggtcgcccttcgtcgtcgtcgacgggtggtcggcgcggccgaccgggagcggcgggagcgggctggtactcccccacttcctgcaatctagccaggtggcacagggtggtcaaagctaaaaagggcggacgcggtttagcttccagcgcaaaggtttcgcgcgttctttcggccggggggcaggtggatcggggcgggctcggggcgaggacggggctgggaatggggcgggggatggggcgggctcggggcgggggtcgcccggagcccgccacgggtcagaggcgcacgcggacgacggtgaacgggaggttcggctcggcgatctggtactggaagttgaccaccagcaggtcgtcgccgtcgaaggtcgcggtggacgggacgtccatgcccctgccgttgacccgccgcagcaccgtggcgcgggagtggtcctcgctcagccgcaggacgctgatctcgccctccgggtggaacaggctggtgacgctgtagaggtcgttgccgcgcaggagcagcccgtccgagccgatgtcgcccacgccgcccaggtcgatcggggtgacggcgccggtgcgggtgctgatgcggtggaacgcctgggagttggtgtcggcgagcagcacgtggcggccgtccggggtgaccacgaggccgttgccgttgatgccctcctcgtagcgcaccggggagtcggcgaggtccacgaacgtcctcagtggctggtcgacctcggggctcgccagctgggcggcggtgatccggtagaggacggggcggaacgagtcgctgacgtaggcgtcgccgttcggggcgatggcgacgtcgttgaccaggccgtcgcgggcgccggagtcgaacacgtgcaggagcgcgccggtgcgggtgctgtggacgaagaccttgccggtggcgccgcccgcgatgaccagcctgcctcgggtgatcttcatgccgacggcggtggtgcggccgtgctgaccggcgggcaggaacggctccagggcggggcggtcgacgtggccgcgccagatcgtgccgtcggtcgtgccgccgacgtagaagtgcggcgtgcccggctcgcggacgatgccctccgggtaggcgcggtcgccggggaccacgtagcgggtgacggggtggtgcgcggcggcggcgggtggcacggcggcggcgggtggcgcggcggcggcgggtggcgcggcggcggggagggctggggcgagcgcggtgaggagcagggtcgcggtgaggagggctcgggtggtggtcacggaagggctccgggggtcgaaggggtgtctggcgccagacaagcgcttcgtggcgcggggtggcagtgggcgcttgtcgggggtagttcttcaccccccttccgggcggggcggccgactagggtgagcggtgtgggcgatcttgggcggcacccggtggcgaaccgctccgacgtgcgggacttcctggtcagcaggcgcgcgagggtgagtccggggcgggccgggctgcccgtgctcgggcggcggcgggtgccggggttgcggcgggaggaggtcgcgctgctcgccggggtcagcgtggactggtacacccgcttggagaaggggcacatcggcggtgtctcgcgggaggtgctcgacgccgtggccggggtgctgcggctcgacgccgaggagcgggtctacctgttcgacctggcgcgcgcggcccggcgtccccgcgccgccgaggtggcggcggaggccgcgctgcccgcgacggcgcagtggctgctggacagcatgacgctgtcgtcggcgatggtgaccgggcggcggcaggacgtgctggcggtcaacccgctggcccgcgcgctctacgcgccgctgttcgccagcgccaccacgcgggacggcggccgggcgaacctcgcccgctaccacttcctcgacgcgggcgcccgcgagttctacggggactgggcgggcaccgccgacgtgctcgtcgccgcgctgcgcgccgaggccgggcgcgacccgcgcgacggggccacccgcgagctggtgggcgagctgacggccgcgagcaccgagttccgggcgcggtggagcgcgcacgacgtgctgctgcacccgcgcggcgccaagaccttccggcaccccgaggcgggtgagctgagcctgagctaccactcggtggacctgccgatctccgccaccgagacccggcacgtgtgcgcgtgcaccgccgaacccggctcgaccgacgaggcgaggctgcgcgcgctcgtcgggtgagccgggggtggccggccaccgccgtcgcgctcgcggcggcggggcggggccgccggtcagagcgtgagcgccatcccgatcgagccgggcgcggtctcggtgaagccgtgcttgaggtacagcgggcggccgggcgcgtcggccaggagggtcacgaacgcgccgggcggggcggcctcgcggatgcgccggagcagcgcgtccatgatcgcgccgccgacgccccttccctggtggtcgggcagcacggccatgtcgacgacgtggaagtaccagccgccgtcgccgaggacccggcccatgccgacggtcccgccgtccgcgtgcgtgacgtggaaggaggcccaggcgccgggcagggcggcggcggcctgctcggcggtcttgggcgacaggccggactcggcgcgcaggcggaggtagtcggcgacggacggcggggtcgggtggagctcgtagtcccggtgcacgcggtcaggctcccacgggcgcgggcgggcccccgcccgacctgacgatttccccgtcggcggggatgcgggcgggcgctcgcggattttcgacatcccccggcccggcgagacgcggcggcgccgctgaaaagagcgccgtcgcggcccttcgcgccgcccccgacatcccccgcgcggcgaccggtcaatgcggtccacgcctgggggtttccctcccacgtcgaacaccgccaccacgcgcccacgcgccgcgtcgaccaccccgacgccgaggaacacctgttcacgggcacgggaagccgcagcggagggggaaccgggaatggccgcaggcgatcgcggcacgacgtccgcacatcaccgcgagcagaatcgcgaggcgttcaccggggcggcggaggaagattccagcgcccctctcgaagaacctgcgggaagccctggaagaaaacccggacccgaaacgcgacaaaattgcggacacccacccgtgaaacaccgggcgcccccaccaggtcacccgctgacatcacgctcagtcagtatcggcacgctcccccgccgaggcggagcgcgacaccccgcccaaccgggcaccgagcgggcacctccactcggcccagcaccgccccaagatcgcacgtagcacgggttgaaaccgctcaagcgcatctcaacccgttcggagcagagtggcgcccgtcacgtcccgaccggtcacggttggcaacgggtccagtccacgcgaggtggcatcaagcgcacttgccccgatcacacccgcccgtgcaaccgaatgcagcagggatatctttcccgagaactcggccgttaaccgggagtggagccaggcccacccctaagacgcttgcccacatgcccaacaatggtgaagatggaacggcggaccgcaccgcgaacgcgaaccgaactccgcgagagggcacggtgaacgatcctggaacagctactgccgcgtagctcaagggtggaacgcccggctcgcgcgcggcggagggaataacggcttttacgccctcgacaacagcttgtcaacgaaacccgtgcacccgagcggtccccgcgcgcaccgctcgcgggggtggcgcggcgcacgacgtggctgcccggcgtcgacgacgacgcgagttccccgaccgcccgcgaaggcggtcgcggatcgccacgacggggcacccggaccacgcctcccccggaacagcgcgcccacgcgcggttccggcgcgcgccgggaccgccgcacccgccggagcgccgccaccggccggggccggtccccgcggaccgggtcgccttccggaccaccactccacggaccacggaaaggaccactcccccagtggagcttctgcgcgcacccgagatccagtcggccgtcgagcacctcgcggtggacctgccggaccgggcgggacgggcgttcctggtggacggaccgcccgcctgcggcaagacgacggccctgcggcggctcgtcgaccggatcgcccacgaggaccacctcgtgctcaccgccacctgcaccccgccggagacggagctgccgttcggggtgctcaagcagctcctcgcctcccccggcatggccagggtcgacccgcgcctggtcgccgacctcggcgagctgctcgccccggccccgccgcccgccgacgactcggcgctcctgcagctgtaccactcgctgtgcgcggcgctgatcgcgctgtccgaggaggtgccgctggtcatcgcggtggacgacgtccgccacggcgacaccgcctcgctgcacgtgctgctgcagctggtgcaccggctggacacggcgcgggtgcggctgctgctcaccgacgacctgctgctgccggtgagcttcccgccgctgcgctacgagctgctgcgcctgcgcgggctcggcctggtccgggtcgcgccgctgcctgcggccagggtgcgggaggaggcggtgcgggcggtcggcgcggacgtcgcgaagcgggtcgacttcgccgcgctgaccggcggcaacccgctgctgctgcacgcgctggcggtggacgtgctggaggcgggcgagccgcgcgagatcggctacggcaactcgttcctgtcctgcctgcaccgcaacgaacccctgttcctggacaccgtgcgggcgctggccgtgctgggcggcggctcggcgtcggacctgggcaggctgtccgggcacgagccggagcaggtcgcccaggtgctgaacgccctgcgggagtcggggctgctggccgaggacgggttccggcacgacgcggcgcgccgcgcggtcgtcgcggacaccccggtcgccgagcacgaggtgctgcaccgccgcgccgcgcggctgctgcgcgaccagggcggcgcggtcaccgacatcgccgaccacctgctgcgggcgggccgcatcaccgacccgtgggcggcggacctgctggtggacgcggcggagctggtggtgcagcgcggcgagccgacggcggcggtggcgctgctccagcgcgcgctcgactgcagcccggaccgggagcgcaggacggccgtgcaggcgcggctggccacggccgagtggctggtgaacccgtcgacctcggcaaggcaccacaccgcgctgctggcggcgttccacgcgggcaggttgtcggtgcgcgacagcgcgacgctgatgaagcacctgcgctgggccgggaacaccgccgactcggacgcggtgctcgcccggctgcggaccgacccgcgcgccgccgaggacgtgccggtgctggagcactggctgaccagcacctaccccggcgcggcccggcccaggaccgtgctggggcgggacgtggactcggcgcgcagcagggcggacctggtgccgagggcgaacgcggtgctgctggacgtgctggtggccggggacagcgacgacgtggccgaccgggcggaggcggtgctgcgggagctgcggctggcgcgggagtccgggtgctacggcggtgcggccgtgctggcgctgtccgcgctgctctactcggaccgcgcggacgtggccgcctcgtggtgcgagcagctgctgtcggcgcgggccgtgccgctgctgccgatgccgcgcgcgcaggtgctggcgctggcggccgagtcggcgctgcgccggggcgaccacccgagcgcggacgagctggcgcgggaggcgctgaccgtggtgtccccgaccgcgtggggggtgtcggtggggctgccgctgagcaccagggtgctggcgctgaccaggatgggccgctacgacgaggcggcggccgtggtggcgcagccggtgccgaacgggatgttcgggcaccgcaacagcgtggactacctgtacgcgcgcgggcacttcttcctggcgcgggaacggccgcgcgcggcgctgggcgacttcctgctgtgcggggagcagctgacccggtggggcctgggcagcgggtgcgcgccggtgccgtggcggaccgcggcggcggaggcgtggctggcgcagggcaaccgggaccaggcgcgggtgctgatccacgagcagctcggcaggccgggcacggacagccgccgggcgcgcgggcaggcgctgcggctgctcgcggcgaccagctcggtgaagcggcacccgcagctgctgcgggaggcggtggcggtgttcgaggccgtcgacgacaagtacgagctggcgcggaccctgcgcgacctggggagggcgcagcgggcgctgggcgagaacaagctggcgcgccgggtgatccggcgggggtggcacgtcgcccggatgtgcgaggcggcgccgctgtgcgaggagctgatgcccaccgccgacgggctggtgcccgcgcagcccgcgtcggcggcccgcaggtcggacctggaccggttgaccagctcggagcaccgggtggccgcgctcgcggcgtcggggctgacgaaccgggagatcgcggtgaagctgtacgtcacgcacagcacggtggagcagcacctgacgcgggtgttccgcaagctcgggatcaagcagcgggagcagctgccgccggagctgagcgtcgaccggtcgaagtgacgcggacggggcggtcccgtggatctggggccgccccgtccggtcccggtccgtccggtcccgccctgtccggtcccgccctgtccggtcccgccccgtccggtcccggtccgcctcaggctcggggcatcgcggccagggtggtggcgacgacgtgctcgtcgacgtcgcgcaccagctcggcgccgcgcgggccgtcgagcacgaacgccaggccgccggtggacttcttgtcgcggcgcatgaaccgcagcagctcgtcgtccggcacgcccggcggcagcgcgacgggcagcccgtagcccgcgaccacggagtggtgctcggccacccggtccgggccgatccggccgagcgcgcccgcgaggcggccggcgaagaccgtgccgatcgcgacgccctcgccgtgccgcaccgcgaaaccggtggcgagctccagggcgtggccgagggtgtggccgtagttgagggtgtgccgcaggccggagtcgcgctcgtcggcggccacgacgcgggccttgagggcgacgctcgccgccacctggtccagcagcggcagccggtccaggcccggcgcgccgatgaagtggcagcgggcgatctcgccgaggccgttgcgcagctcgcgctcgggcagggtggcgagcaggtcgaggtcgcacagcacggcggcgggctgccagtaggcgccgacgaggttcttgccctcggggaggttgaccgccgtcttgccgccgacgctcgcgtcgacctgggccagcagcgaggtcggcacgtgcaccaccggggtgccccggtggtagagcgaggcggcgaggccgaccgcgtcggtggtggtgccgccgccgcaggagacgacgacgtcggcgcgggtcaggccgaactcggcgaaccggctgcacaggtgggcgacggtggcgagggtcttgtcgtgctcgccgtcgcgggccgggaggacgagggaggggacgccggggtcgggcgtctggtccgccgggcgggcggtgaccacgacggcgcggcgcgcgccgagggcccgcacgacgtccgggagggcggcgcgcacgccgtgtccgatgtggacggtgtaggcgcgctcgcccagctcgacccggacctcgcgggtggtggcggcggtgggggcggggtgggtggagctgggcactgcttcctcctcgggtgggcgggacggggggcgatcgggggacgcggaggggtgacgggaaagcaatcgggcaggaatgggaacgggtccgggggcgaacgggcaggaattcgaatgggggcaagcgaccgggagcgatcccagtggtggggcggaagtgcgggcggcggaaaggcggtcgtgctgcctcagccgccgcccgcggcgcccgtcacgagcgtggtgcgcagggtgagcgccgcccgcgccgccacggccgcgccgacgccgaggcggttgtgggtcagggtcgcctgctcgaacagcaccggcagggccgggcggcggcggtccgcggcggcgcgcagctgctccaggtagcgccgccacgcgcccgccgagccgaccacgcgcccgccgggcggcgccccctgcgcggcgacggcggcctcggtgcgctcgaccgggcggagcggcctgctccagctctgccagcagtggaacaggaacgcgggcctggccggttccggggccagcgcgaccagttcggccaggtggtgcagcaccgtggcctgcgcgcccgactcgccagggtcctcgccccacaccgggctcaccaccgacgcgacgtccagggccagttcgctggacgcggtcgcgaccagcggctcgaccggcccgcccggcaggcccccacccggcaggccctcgcccgccgggtcgacgcgcaggtcgcgggccgccgcctcggcgcacagcacggcgagcaccgggccccgcgcggcctcggtcgtgcccagccacagcgccaccacccccgccccgcgcaggaacgaccagtgcgcgccccggtcccgcgcgcgcagctcccgcacgacggggggcagcacctcggccaccgagcgggctccaccgcctgccagcgaccagcccgaccaggacggggcgcccgccgccgggggtccgccgaggggcgctctcgcggaaccggtccgcgtcatgtccaccaccacttccgccttggcgagaacgggtcctgcgggatcaccgcgctgttccgacgccgccgacaatagcgacgcgcaatacgccgaattcaccgccaaatcaggtcaggggggttgagggggatgccttagggggcgagtgcccgcaaagcggaagaagaatcggaagcacatgcaggagcgacttccaagctcaggccgcaggaccgggtccgcgtcgtcgcggacaccccggtcctgcgcgtgcgcgcaccgaaggacgtggtgacatgcttcggaccgacctgatccgcccggttcccgaactgctcggggccaacgcggatcgcttcggcgacaggaccgcctactcggacggtcgccgttcggtcgggcacgccgggctggaacggcgcacgcgccgcctcgccggtcacctcgggcagttgcggctgcaccccggcgaccgcgcgatgatctgcctgggaaatcgcgtcgaaatgatcgagagctatttcggcgtgctccgcgcggacgccgtggcggtcccggtgaacccgcgttccaccgacgcggagctgacccacctgctcgccgacagcggggcccggctggtgatcaccgacgcggcgcgcgccgagcggttcgaccggttgcgcgccgagcggttcggcgacctgaccgtgatcgccacccaggacggcccgctgcccgacggcgtcatcgcgttcgagccgctggccgccgaggagccggagctgcccgcgcgcgacgggctcgggctcgacgacgtggcctggatgctctacacctccggcacgaccgggcgccccaagggcgtgctgtccacgcagcgcagctgcctgtggtcggtggccgcctgctacgtgccggtgccggacctgcgcgccgaggaccgcgtgctgtggccgctgccgctgttccacagcctgtcgcacatcacctgcctgctggccgccacggccgtgggcgcgaccacgcgcatcgtggacggcacgtccgcgcaggacgtgctcgcggcgctggagcaggagcggtcgacgttcctggcgggcgtgccgacgctgtaccggtacctggtcgacgccgcccgcgagcgcgggttcaccgccccggacctgcgggtgggcctggtcggcggggcggtgacgacggcggagctgctgcgcgcgttcgaggacacgttcggcgtgccgctgatcgacgcctacggcagcaccgagacgtgcggggcgatcgcggtgaactggccgaccggggcgcgcgtggcgggctcgtgcgggctgccggtgccggggctgacggtgcggctggtggacccggagacgctgctggacgtgcccgccgggcgggagggcgagttctgggtgtcggggccgagcgtgatgctgggctaccacaaccagcccgaggcgacggccgaggtgctgcgggacggctggtaccgcacgggcgacctggggcggcgcgacgaggccgggttctgcacggtcaccgggcggatcaaggagatgatcatccggggtggggagaacgtgcaccccggcgaggtcgaggccgtggtgcgggcggtgccgggggtggcggacgtcgccgtcgtgggcaagccgcacgacgtgctgggcgaggtgccggtggtgttcgtggtgccgggcgcgggcgggttcgacccggcggcggtgctggcggcgtgccgggaggagctgtcgtacttcaaggtgcccgaggaggtctacgagatcgagcgggtgccgcgcacggcgtcgggcaagaccacccggcacgtgctgctggacctgcccgcccggttgcgggcggcgtcgagcgggcagttccagtcgctgctgcggctggactgggtgccgaggacggcgctgccgggtgaggaggtcccggcgagctgggtgctggtggacggcgacccgctggggctcgcggacgggttgcgggccacgggcgcgcgggtgcgggtgggcgagccgggcgcggatgcgctgggcgacggcggatcggacgccgacgagccgggcgcgagcagcgcgggcgaaccgggctcgggtggctcgggtgagccgggctcgggtggctcgggcgaaccgggctcgggtgaaccgggcgcgagcagcgcgggtgagccgggtgcgggtgagccgggcgcggccgaacccccgcaggtcgtgctggtcgccgcggtccccggtgagcgtggtgaggtcgcgcgggacgtggaggcgctcgcggacgggctcgcgcggcggctcgtcgggtggctggccgacgagcggttcgcgggggcgcggttcgtcgtggccacctcgggcgcggtgtcgacctcccccggcgaggacctgcgggagctgcgggcggccccgctgtggggtgtggtgcggtcggtgcaggccgcgttccccggtcgggtggtggcggccgacctggacgcgtccggcgacgggcgggcggcggcgctggctcgcgtcgtcgcgggcgggcacgaccaggtggccgtgcgcggcgacgtgccgctggcgccccggctggccagggtgtccgtgccgtccgacccggcccccgccccggcgctggacccggacgggctggtcgtggtcaccggtggcgactcggcgcgcggcgcggccctcgcgcggcacctggtggccgcgcacggcgcgcggcgcctgctgctggtctcccccgacgggctgcccgaccaggccgccgccgacctggaggccgggttcgcggcggcgggcgcgcgggcggagtcggtggtgtgcgacccggccgacccggtcgcgctgcgcgccctgctcgacgcgcaggaccgcccggtcacggccgtggtgcacgtgcagggcgggcgggcgctgctggactcggcgcgcgccctcgtcgccctgcacgagctgacccgccaggcgcgaccggcgctgttcgtcgtggtcacctcggtggccgggctgctgggctcggcgggcgacccggcgcgcgcggcggccgaccagttcgccgaggcgctggtgcgcaggcgcgccgaccggggcctgccggggctggccgtggcctggggtccgctgccgggcgagcccgcgcaggcgggcgcgggcgcgctgccgatggccgaggcgctgaccctggtcgacgccgcgctcgccgccgaccagggcccgctggtggtgctcgggctcgacgcggtcgggtcgcggcgcgcggtgggcgcggtgccgccggtgctgcacgacctggtcgacggcggtcgcgccgcgcgggtcgcgccgggcgcggtggccgagttcacgcgcaggctcgcggaggcgggtgggcagcgggcccgacgcgtcgcgctggacctggtgcgcgagcacgtcgcggcggcgctcggcctgcccgaggacaccccggtgcgcgccgaccaggcgttccgcgacttcggcgtcacctcgctgaccgccgtggcgctgcgcgaccggatcaacgccgcgaccggcgcgtccctgcccgcgacggcggtgttcgaccacccgaccccggccgcgctcgccgaccacctggtgcgcgaggtcaccggcgaccggccgcacgtcgagcgggcgcgggacgagcgggcgcgcgggacctcgcgcgcggacgagccggtggcgatcgtcgccatggggtgcaggctgcccggcggcgtggcctcgccggaggacctgtggcggctggtggacgagggcgtcgacgcgatcggcccgttcccgaccgaccggggctgggacctggccaccctgctcgacggctcggactcgccggggaggtcctccgtggaccgcggtggtttcctgccgggcgcgggcgacttcgacgccgggttcttcggcatctccccgcgcgaggccctggccatggacccgcagcagcggttgctgctggaggtggtgtgggagaccgtggaacgcgccgggatcgacccgcgctcgctgcacggcgaagacgtcggcgtgttcagcggcctgatgtaccacgactacgggaccgaacccggttccgcgccggagggcctggaggggttcgtcagcaccggcagcgcgggcagcgtggtctccggccgcgtcgcctacgcgctcggcctgaccggcccggcgctgaccgtggacacggcgtgctcgtcgtcgctggtggcgatccacctggcggcgcaggcgctgcgctcgggcgagtgctcgatggcgctcgcgggcggggtcgcggtgatggggcagccgacgtcgttcgtggagttctcccggcagcgcgggctcgccgccgacgggcgctgcaagtcgttctccgacgacgccgacggcacgaactgggccgagggcgtgggcgtgctgctgctggagcggctctcggacgcgcgccgcgacgggcacccggtgctggcggtgctgcgcggcagcgcggtgaaccaggacggggccagcaacgggctgaccgcgcccagcggcccggcgcagcagcgggtcatcaggcaggcgctggcgaacgccgggctgcgaccgtccgaagtggacgccgtggaggcgcacggcaccggcaccaccctgggcgacccgatcgaggcgcaggcgctgctcgccacctacgggcaggaccgcgagcagccgctgtggctgggctcgctcaagtccaacctcgggcacgcgcaggcggcggcgggcgtcgcgggcgtgatcaagatggtgatggcgctgcggcacggcgtcctgccccgcaccctgcacgtcggcacgccctcgtccaaggtcgactggtcggcgggcgcggtcgagctgctgaccgaggccaggccgtggcgcgcgaacgggcggccacgccgggcgggcgtgtcctcgttcggggtcagcggcaccaacgcgcacgtcgtggtggaggagcaccgggaaccggccgccgcgccggtcgacccggtctcccccggcctggcggtcagcggcggcgtcgcgccgctggtgctgtccgggcgcacccgctccgcgctcgccgcgcaggccgcggccctgctggggcacctggccgacgggaccgacccggcggcgctgggccgcgcgctcgccaccacccgcaccgcgttcgagcaccgggccgcggtcctcgcgccggacgtcgacgccgcgcgcgccggggtgcgcgcgctcgccgaggaccggcccgcgccgaacctggtcaccgggcaggccgacgtggacggcccggtcgtgttcgtcttccccggccagggcgcgcagtggaccggcatgggccgggagctgctggagacctcgccggtgttcgccgcgcggctgcgcgagtgctcggaggcgctggagcggtggaccggctggtccctgctcgacctgctcgccgacggggcggagctggaccgggtcgacgtgctccagcccgcctcgtgggcggtgatggtggcgctggccgcgctgtgggagtcgtgcggggtgcgcccggacgccgtggtcgggcactcgcagggcgaggtggccgccgcgtgcgccgccgggtggctgtcgctggacgacgcggccagggtggtggcgctgcgcagccgcgcgatcgccgagcacctggccgggcgcggcggcatgatgtccgtcgccgccggggcggagcgggtggccgggctgatcgccgaccggcagggccgggtgtcggtggccgccgtgaacgggccgtccgcgaccgtggtggccggggccgccgacgcgctgcccgagctggccgcgcgctgcgagcgggagggcgtgcgggcccggatcatcccggtggactacgccagccacaccgagcacgtggacgcgctcgacggggtgctgcaggaggtgctggcgggcgtcaccgcgcaggccgggcacgtgccgtggctgtccaccgtggacggcgagtgggtcgacggctcggggctggacgcggactactggttccggaacctgcgcgggaccgtgcggttcgccgacgcggtggcggcgctggcgggctccgggcaccgggtgttcgtggaggtgtccagccacccggtgctcaccgccgcgaccggcgaggtgctggaggccgccggggtgcgcgacgcgctggtggtcggctcgctgcggcgcgacgacggtggccccgagcggttcctcaccgggctcgccgagctgcacgcgcgcggcgtcccggtggggctggaggcggtgttcgcgggcgcggacgggcgggtggagctgccgacgtacgcgttccagcacgagcggtactggctggcgcgcggcccggtggccggggacgtgtccgggtcggggctggtggacgcggcgcacccgctgctcggggcggtcgtgccgctgccgggcacgggcggggtgctgctgtccgggcggctctcgcaccggcggcagccgtggctggccgagcacgcggtggccgggacggtgctgctgccgggcgcggcgatcgtggagctggccgtgcgcgcgggcgacgagaccgggtgcggggtgctgcgggagctggtgatcgggcagccgctggtggtgccgccggacgccgaggtggacctgcaggtgctcgtcggcggcccggacgacgggggcgtgcgggacctgcggctgtactcgcggaccggggcggcggcggagtgggtcgagcacgcggcaggcgcgctcgcccccggcggcgcggtcggcggggcgcgaccggccggggcgcggacggccggggcgcgactggacggggcgcgactggacggacagtggccacccgcgggcgcggaacccgttgcgctggaaggcttctacgagaacctggcggagctgggctacgagtacgggccgctgttccgggggctcgcggcggcgtggacgcgcgacggcgaggtgttcgccgaggccgtgctgcccgaggaggcgttgtccgggcaggcgttgtccgggcaggcggggtccgggcaggcggggtccgggaacgggtccgggaacgggttcggcatccacccggccctgctggacggggcgctgcacgcgggcaacctgtgcgtgccgcccgcgccgggccggacgctgctgccgttcgcgtggaacgaggtgcggctgcacgccaccggggcgacggcggtgcgggtgcgcgtgcgggcgaccggcgaggactccctggagctggagctgttcgacgccgacggcgcgcccgtggcgagcgtcggcgggctgaccctgcgaccggcggtcacgggcgcgcgcccggccgagtcgctgcacgaggtggagtggaccgaggtcgcggcgggcggttcgtggccggaggtcgccgacacccgcgactgggaggccgccgccgacctgccgacccggtcgcgcgagctggccgcccgcgcgctggaactggtgcaggaccggctggcgggcgtggacggcgcaccgctgctggtgatcaccacgggcgcggtggcggtggccgacgacgccgaggtcaccgacccggccgccgccgccgtctgggggctgctgcgctcggcgcagtccgagcaccccggccggttcgcgctggtcgacgtcgacggcggcgcggcggccgaggtcgccgcgctcgtgcccggcgacgagccgcagaccgcgctgcgcggcgggctcgtgcgggctccgcgcctgcgccgcctgccccccggtctcgtgccgcccgccggggcgcactggcacctggacgcagtcaccaccggcacgctcgacgggctcgcgctcgtggcctcggaaccggtcccgctgcgggccggggaggtgcggatcgaggtcagggcggccgggcagaacttccgggacgtgctggtggcgctggacggcgtcgcgggccaggagggcatcggcggcgagggctccgggatcgtgaccgaggtcggccccgaggtgaccggattcgccgcgggcgaccgggtgatggggctgttcccgcgctcgttcgggccgctggccgtggccgacgcccgcacggtggtgcgggtgccgcgcggctggtcgttcaccgacgcggcggccgtgccggtcgcgttcctgaccgcgctgcacggactccaggacgtcgccgggctgcgggccggggagacggtgctggtgcacgcggcggcgggcggcgtcgggcaggccgccgtgcagctcgcccaccacttcggcgcgcgcgtgctggccaccgcgcacccggccaagcacagcgtgctgaccgcgctgggcgtgcccgccgagcggctcgcctccagccgcgacctcggctacgcgcggcggttcggcgacgtcgacgtggtgctgaactccctggtcggcgagcacgtcgacgcctcgctgcggctgctgcgcgcgggcggccggttcgtggagatcggcaagaacgacgtccgggacgccgactcggtcggggacgtccgctaccgggtgttcgacctgggcgcggacgccgggccggaccggatcggcgagctgctggagcagctggtgggcctgttcgagtcgggcgcgctgcggccactgccggtgcgcacgtgggacgtcacccgcgcggcctcggcgttccgcgagatgagccggggcgggcacaccggcaagatcgtcctgacgatcccgcgccgcctcgaccccgagggcacggtgctgatcaccggcggcgccggcacgctcggggccaccgccgcccgccacctggtcaccgcgcacggcgcgcggaacctgctgctggtcggcaggcggggccccgacgcgcccggcgcgagcgagctggcggaggagctgcgcgggctgggcgcggacgtgcgggtggcggcgtgcgacgtcgccgaccgggccgcgctcgacgccctgctcgcctcggtcccggccgggcgcccgctgacggcggtcgtgcacgcggcgggcgcgctcgacgacggcacggtcaccgcgctcaccccggagcggttcgacgcggtgttccgccccaaggtggacgcgatcgcgcacctggacgaggcgacccgcgacgccgacctggccgcgttcgtcgtctactcctcggcggcgggcgtgctcggcaacgcggggcagggcaactacgcggcggcgaacgccgtgctggacgcggtggcccgcacccggcacgcccgcgccctcccggcgacctcgctggcctgggggttgtggagcgacacgagcgcgctgaccgcgacgatggacgggcgcgcggtggaccgcacgcggcgcgcgggcgtgctgggcatgggcaacgacgaggcgctggcggcgctggacgcgggcctggcgtccgggctgcccgcgctggtggccgcccggatcgacccggccgcgctgcgcgaccccgcgtcggggtcgccgctgctgcgcgggctggtgcgcgccacccgccgcacggccgccacccgcgaccgggacgccgtgggcgggctggccggacggttggccgggttgtcggccgcggagcaggacgagctgctgctgggcctggtgcgcagcgaggccgccgccgtgctcgggcacgcgagcgccgagcgggtcgagccgcaggtggcgttccgggacatggggttcgactcgctcaccgccgtggagctgcgcaaccggctcgcggcggcgaccgggctgcggctgcccgcgacggcgacgttcgaccacccgacgccggtgcggttcgccgcgctgctgcggggcgagctgctgggcgccgtcgtggctcccggagccgtgaccgccgccgcggctcccgtgaccgccgccgcgcccgccgacgagccgatcgcgatcgtgtcgatggcgtgccggctgcccggcggggtggtcgacccggccgggctgtgggagctgctcaccggggagcgggacgggatcgtggacttccccgacgaccggggctgggacctggagtcgctctaccacccggacgccgactcccccggcacctcctacgtgctgcgcggcgggttcctggacgacgcgggcgggttcgacgccgggttcttcggcatctccccgcgcgaggccctggcgatggacccgcagcagcgggtgttcctggagacctgctgggaggcgttcgagcgcgccgggatcgacccggtctcggtgcgcggcagcgacaccggggtgttcgccgggatcatcgaccaggactacggggtgcgcgcgggcacggcccccgaggagctggagggctacctgctcaccggcaccgccacgtcggtggcgtccgggcgggtggcctacctgttcgggctggagggcccggcggtcaccgtggacacggcgtgctcgtcgtcgctggtggccacgcactgggcggtgcaggcgctgcgccggggcgagtgctcgatggcgctggcgggcggcgcgaccgtgatggggcggccgtcggcgttcgtggagttctcccggcagcgcgggctggcgcgggacgggaactgcaaggcgttcggcgcggacgcggacggcaccgcgttcagcgagggcgcgggcgtgctgctgctggagcggctctcggacgcgcggcggcgcgggcacccggtgctcgcggtgatccgggggtcggcgctgaaccaggacggggcgtcgaacgggctgaccgcgcccagcggaccggcgcagcagcgggtgatccgggcggcgctggccgacgcgggcctgcggccgtcggacgtggacgcggtggaggcgcacggcaccggcaccgcgctcggcgacccgatcgaggcgggcgcgctgctggcgacctacggcgcggaccgggagggcgcggaaccggtgtggctggggtcgctcaagtccaacaccgggcacacgctggcggcggcgggcgtgtcgagcgtgatcaagatggtgctggcgctgaaccacggcctgctgccccggtcgctgcacgtgcgggagccgagcgcggcggtggactgggagtcgggcggcgtgcgcctgctgacgagcgcccggccgtggccggagagcggcaggccccggcgggcgggggtgtcgtcgttcgggatcagcggcacgaacgcccacctggtgctggaagccgcgcctgcggaggagggcgcgggggcgcggagtggggcggcggcgccgggaccggacacccggtcggcgcccaccccggacgccccagcgggccccgtccagacctccggcgtgatcccctggccgttgtcggcccgctccgccgacgcactgcccgcgcaggccgcgaagctggccgcccacgtgcgggcgcacgacgacctctcgccgctcgacgtcggctggtccctcgcgaccacccgcaccgcgcacccgcaccgcgccgtgctcgtcggcggcacccgcgaggcgctgctgtcggccgccgacgcgctcgcgggcggcgaggccagccaggccgtgctcaccggctccgccgtcgggtcgggttcggcgaagaccgtgttcgtgttccccggccagggcgcgcagtgggcgggcatgggccgtgagctgctggggtcctcgccggtgttcgccgcgcggctgcgcgagtgcgccgacgcgctggccccgcacaccgactgggacctcctggacgtggtgcgcggcgcggagggcgcgccggggttcgagcgggtcgacgtgctccagcccacctcgtgggcggtgatggtggcgctggccgcgctgtggcgctcgtgcggggtggagccgtccgccgtcgtcgggcactcgcagggcgaggtggccgccgccgtggtcggcgggtacctggcgctgggcgacgcggcgcggctgatcgcgcggcgcagcagggccatcgcgcaggagctgaccgggcgcggcgggatgctgtccgtgctcacctcgcccgagcgggtcgccgaactgctggagccgtgggccgggaagctgtggatcgcggcggtcaacagccccgcgtccgtctcggtgtccggtgacgccgaggcgctgggcgagttcgtgcgggtgctggccaaggcccggatcaaccggtggcggctgcccggcgtggacttcgccgggcactccgggcacgtcgacggcatcgaggcgcggctgcgcgaggagctggccgacgtcaccgccgcggcgggcgaagtgccctggctgtccaccgtggacgggcggtgggtggagcgcaccaggctggacgccgactactggtaccgcaacctgcgcgacgtggtccgcttcgacgaggccgtccgcgcgctggtggacgccgggcaccgggcgttcgtggaggtctccacgcacccggtgctgaccaccgcgatcggcgaggtcgccgacgagcggcaggacgtgcgggtcgccgtggcgggcacgctgcgccgcgacgacggcggcgcggaccgggtcgtgggcgcgctcggcgaggtggccgcctcgggcgtggcggtggactgggcggcggtgttcggcgggaccggggccgcggtggtggagctgccgacgtacgcgttccggcacgagcggttctggctcaccccgtccggcggcgacgtgcgcgcggtggggctgcggcaggccgggcacccgctgctgggcgcggtggtcagcgtcccggacaccggcggcgtgctgctgaccgggcggctgtcgctgtccgcgcagccgtggctggccgaccacgcgctgtccggcgtgccgctgctgccggggacggcgctggtggagctggcggtgcgcgcgggtgacgagaccggcacgccggtggtggcggagctggtgctgggcaggccgctcgtgctgccgcgcaccgggtcggcgcaggtgcaggtgctggtgggcgaggaggcgcgggacgggcggcggccggtcgcggtgtactcgcgggcgggcgacgaccggccgtggaccgagcacgcctcgggctcgctcgcgccggacgaggacgccgcgccgggagcggagggcgacgagtggccgcccgccggggccgagccggtggacctcggcggcttctacgacggcctcgccgaacggggctacgactacggcccggccttccggggcctggtgcgcggctgggtcaggggcgacgaggcgttcgccgaggtcgggctgcccgacgaccagcacggcgcggcggcccggttcgggctgcacccggcgctgctggacgcggccctgcacgcggcctcgctgtgcgcgggccacggccggggcacggcgctgccgttcacctggaccggcgtgcggctgcacgcggccggggcgacggcgctgcgcgtgcggctggaggcggacgggccggagcggttgtcgctgcgggcgagcgatccggcgggcacgcccgtggtgaccgtcgggtcgctgctgctgcgcgccgccgacgcggaccggctgcgggcgacagcggcggcgacggcggcagcggcggcggacgacgggctgcacgcgctggagtggaccccgcacccgctgcccgaggagacgaccggttcccccgccgtcctggacaccagggcgtgggagctgcccgagggcgtcgggcgggccgaggcgatcaccacgcgggtgctcgccgagctccaggccgagctcgacgggacggcgaccctggtcgtggtgacgcggggcgcggtggccgtgcatgacgacgccgaggtcaccgacccggccgccgccgcggtgtgggggctggtgcgcgccgcgcaggccgaggaacccggacgcgtcgccgtggtcgacgtcgacgacgcctccgaggccgcgctggacgccgccgcgcacgccgcgggcgcagaaccgcagctcgccctgcgcggcggggcggcgttcgcgccgaggctggtcgaggcgtccggggcgctggccgtgccggacgggccgtggcggctcgacagcaccggccggggcaccctggagaacctggcgctcgtgcccaaccccgccgccggggcgccgctcgcgcccggtcaggtgcggatcgtggtgcgggcgggcggcctgaacttccgggacgtgctgatcgcgctcgacgcctacgagtcggagatcggcaccgagggcgcgggcgtggtcgtggaggtcgcgccggacgtcacccgcgtggccgtgggcgaccgcgtgatgggcatgatccccggctcgttcgggccgctggccgtggccgacgcccgcacggtggtgcggatgccgcgcggctggtcgttcaccgacgcggcgggcgtgccggtcgcgttcctgaccgccctgtacgggctgcgcgacctcggcggcctggcggagggcgagaccgtgctggtgcacgcggcggcgggcggcgtcggcatggccgccgtgcagctcgcccggcacttcggcgcgcgcgtgctgggcaccgcgcacccggccaagcacgccgcgctggacctgcccgccgaccacctggcctccagccgggacctcgcctacgcgcagcggttcggcgacgtcgacgtggtgctgaactccctggtcggcgagcacgtcgacgcctcgctgcggctgctgcgcgcgggcggccggttcgtggagatgggccgggcggacctgcgcgacgccgacgaggtggcgcgcgagcaccccggccgcgcctacctcccgttcgacctcggcggcgacgccgggccggaccggatcgccgagctgctggtggagctggtggccctgttcgagtcgggcgcgctccgcccgctgccgacccggcgcaccgacctggtgcgcgcccccgaggcgttccgggccatgagccagggccgccacgtcggcaagctcgtgctcaccccgccccgcgcgctcgaccgcgacggcacggtcctgatcaccggcggcacgggaaccctcggcgcggctctggcccgccacctggtggacgcgcacggcgtccggaacctgctgctggtcagccgcagcggccccaacgcgccgggtgcggccgacctggtcgcggagctggccgagcggggcgcgagggtccgggtggccgcgtgcgacgtggccgagaaggacgcgctcaccgcgctgctcgcctcgatccccaccgggcgcccgctcaccggcgtcgtgcacgcggcgggcgcgctggacgacggggtgctcaccgccctggacgccgaccgggtcgcggcggtgctgcgccccaaggccgacgccgccctgctgctgcacgaggccaccgaggacgccgacctcgcgctgttcgccctgtgctcgtcggtggcgggcgtgctgggcaacgcgggccaggcgaactacgccgccgccaacacctacctggacgcgctggcccagcaccggtcggccgccggtctggccgcgctgtcgctggcctggggccggtgggcgcagaccagcgccctcaccgcagacctgcccgcgcccggcggtcgccgcgacctggtgcgccccatggacaccgcgtccgcgctgcgcctgctcgacgccgcgctccgcaccggacgctcgacggtcgtcgccgccgagctggacgtcacggcggccaccgccgcgaacccggtgctgcgcggcctggtccggcccgcccggcgcgcgctggccacgtccgcgcgggacgagcgcggcgtggcggcggcgctggccgggctgggcgaggccgaccggcgccggttcgtgctggacctggtgcgctcgcacgccgccgtcgtgctgggcctggcgggcaaggaggccgtggacgccgagcgcgcgttcaccgagaccggcttcgactcgctcaccgccgtggagctgcgcaaccggctcgccgccgcgaccgggcttcggctgccctccacgctggtgttcgaccacgccaccccgaccgcgctggccgcgcacctgcgcgccgagctgaccggcgacgacctgccgcaggcgcgggccgtcgccgccacggcgggggcgcgggacgacgacccggtggtgatcgtgtcggcgagctgccgcctccccggcggcgcggactcgccggaggcgctgtgggagctgctggagcggggcagggacgccatcaccccgttcccgcgcgaccggggctgggacctggaggcgctctacgacgccgacccggaccggccgggcaagagctacgtgcgcgacggcgggttcctcgccgacgcggccgggttcgacgccgagttcttcggcatctccccgcgcgaggcgctggccaccgacccgcagcagcggctgctcgccgagacctcctgggagctgttcgaacgcgcgggcatcgccccgacctcggtgcgcggcagcgacgtcggcgtgttcgcgggcgtgatcaaccaggagtacggcgtgcacagcggcacgacccccgccgagctggaggggtacgtgatgaccggctcgaccaccagcatcgcctccggccgggtggcgtacctgctcgggctgaccgggcccgccgtcaccgtggacaccgcgtgctcctcgtcgctggtggcgatccacctggcggcgcaggcgctgcgctcgggcgagtgctcgatggcgatcgcgggcggcgcgacggtgatcgcgaggccgggcgggttcgtctcgttctcccggcagcgcggcgcggcccccgacgggcgctgcaaggcgttcggcgacggcgcggacggcatggcgttcgccgagggcgtcggcctggtgctgctggagcggctctcggacgcgcgccgcaacgggcacccggtgctggcggtcgtgcgcggcacggccctgaaccaggacggcgcgtccaacggcctgaccgcgccgaacgggcccgcgcagcagcgggtgatccggcaggcgctggccaacgccgggctgtcccccgacgaggtggacgcggtcgacgcgcacggcaccggcaccgcactcggcgacccgatcgaggcgcaggcgctgctcgccacctacgggcgggaccgggacccgcggcggccgctgtggctggggtcggtgaagtcgaacatcgggcacacccaggcggcggcgggcatcgcgagcgtgctcaagatggtgctggcgatgcagcggggcgtgctgcccgcgaccctgcacgccgacaccccgacgacgaaggtcgactggtcctcgggcgcggtggcgctgctgtcgcgggcgcggccgtggccggagaccgggaggccgcgccgggcgggcgtgtcctcgttcgggatctccggcaccaacgcgcacgtgctgctggagcaggccccgcaggacgcgcccgccacgccggtcgccccgcggggcgccgggctggtcggggcggtggcctggccggtgtccgggcgcacgcccgccgcgctgcgcgcccaggccgccaggctcgggacgcacctggcgggcgcgcaggccggacccgccgacgtgggctggtcgttggcgggcacgcggacggcgttcgcgcagcgggcggtcgtggtggccgggacggcggagcaggcccgtgacgggctggcggcgctggccgaaggccgctcgtccgcgctcgtgacgaccggtgaggccggggtcgacgggcgcgtggtgttcgtgttccccggccaaggggcgcagtggatcggcatgggcgcggagctgatcgacgcgtcgccggtattcgccgagcggttgcgcgagtgcgcggaggcgctggaaccgttcgtggacttcgacctgatcgaggtgctgcgcggacgcgggtcgctggagcgggtcgacgtggtgcagcccgcgtcgtgggcggtgatggtgtcgctggcagcgctctggcggtcgctgggcgtggaaccggacgccgttgtcgggcactcgcagggcgagatcgcggcggcggcggtcagcggggcgctcagcctgcccgacgccgcagccgtggtcgcgttgcgcagcaaggcgatcgcccaggacctggccgggctcggcggcatgatgtccgtcgccctgcccgccgacgacgtcgacctgagcgggtatcccggacgcctgtgggtcgccgcgcacaacggccccacctcgaccgtggtggccggtgacgtggacgcgctgcgcgagctccacgcccactacgagggcgccgaggtccgggcccggatcatccccgtcgactacgccagccacaccgggcacgtcgacaccatccgcgagcggctcgccgaggcactggcgcacgtgcggccgagggcgggcacgatcccgtggctgtcgaccgcgaccggcgagtggaccaccggtgaggacgccgacgccgactactggttccgcaacctgcgcggcgcggtgggcttccacaccgccatcaccaccctcgccgagcagggccaccgggtgttcgtggaagtctccagccaccccgtgctcaccaccgccatcgaggccacgctcgaaggaaccggacccaccgccgtcaccggaaccctccgccgcgacgacggcggccccgaccgcctcctcaccagcctcgccaccctgcacgtgcgcggcgtccacgtcgactgggacgcggtctacgcgggcagcggcgcgcaccgcacgacgctccccacctacgcgttccagcacgagcgctactggctcaccgagccggacgcgccgcaggccgtcgcggacgccccgttctgggacgccgtggacagcggcgacgtggccgcgctcgccgggtccctgggcgtcgagcccgccgccctggagccggtgctgccggggctgacgagctggcgggcccgcaaccgggacggcgcggccgtggacgactggtcctaccggatcggctgggagcgggtggacgtgcccgccgccccgctgtccgggacgtggctggtcgtggtgcccgaggcactcgccgacgacacctcggtcgccgaggtcgcggcggcgctggccgcgcgcggcgcgacgccgaggatcgtggcggcgggcccggacctgggcccggacctgggtgacgagccggacggggtgctgtcgctgctggcgtgggacgaccgcccggccgggggcggcacgctctcgcgcggcgtcgtggacgcggtcgggctggtgcgggaggcggtgcggcgcggctggtcggccccgctgtggtgcgccacgctcggcgcggtcgccgtcgccgaccccggcgaggtgacggccgagttcgggccgcagctgtggggcacgggcgtcgtgctgggcctggacctgccggacacctggggtggcctggtcgacctgcccgcgcggccggacggggtcgcgctggacctgctgtgcgcggtggtcgcgggcgcgggcgacgaggaccagctggcggtgcgcccggccggggtgttcgcgcggcgcatgacccgacgcccggtcgcgtcggcgcccgcgtggcgaccgcgcgggacggtgctggtcaccggcggcaccggcggcctcggcggctacgtcgcccggtgggcggcggagcggggcgcgcgggacgtggtgctgctctcgcgcggcggcccggacgcgccgggcgcggacgccctggtcgccgacatcacggcggcgggcgcccgctgcgcggtgctggcctgcgacgtcaccgaccgggacgcgctggccgaggtggtcgcgaacctgccggacgggccgctgtcggtggtgcacgccgcgggcgtggcgcgaccgggacggccgctggtggagaccacgccggaggagttcgcggccatcggccggggcaaggtcgcgggcgcccgcctgctggacgagctgctgggcgaccgggagctggacgcgttcgtgctgttctcctccggcgcggcggcctggggcagcggcgggcaggccgggtacgcggcgggcaacgccttcctggacgggctcgcgcagcgcaggcgcgcccgagggctcgcggccacctcggtggcctggggcgcgtggggcggcgtcggcacggtcgacgaggtgctgggcgagcagtggcggcgcgccgggctgctcaccatggacccgcgcctggccaccctcgccctcgcgcacgccgtgggctcgggcgaggcgcacctgctcgtcgcggacgtcgactgggcccgcttcgcccccgcctacgcgctggccaggccgcgcccgctgctggcggcgctgcccgaggtcgccgacgcgctggcggtcgtggacgcgcccgccgacgccggggggatcggggcgcggctggccgggctgccgcccgccgagcaggagcggctgctcaccgagctggtgcaggcggaggcggcggccgtgctgggcctgggcggcatcaccggcgaccgggcgttccgggaggtcgggttcgactcgctcacggccgtggagctgcgcaaccggctcggcgcggccacgggtctcaccctgcccgcgacgctggtgttcgaccacccgcgcccgagcgccctggccgcgcacctgcggtccgcgctgggcccggccgccgcgccggtggactcggtggcgggcgtgctggccgagctggaccggctggaggcggccatcccggcgctgccgtcggccgagatcggccggtcccggctggagctgcggctgcggcggttgagcgcccgcgtcggcgagctggtcgccgcgaacggcgagcgggcgaacggcgggcgcgcgaacggcgggcgcgcggcggccgacgagctggacgacgcgggggccgaggacgtgctcgcgttcatcgaccgggagttcggggacgcgtgagcggccacacgagccccgaccccggcccccaccgcggcccccacaacgacgaccctggcgaggaacagatggcgaacgacgagaggctcctcagctacctcaagcgggtcaccgccgacctgcaccgcacgcgggagcggctgcgcgaggcggagtccggggcggacgagccgatcgcgatcgtcggcatggcctgccgcttccccggcggcgtgcgcaccccggacgagctgtgggagctggtggcgtccggccgcgacggcatcggcccgttcccggacgaccggggctgggacctgggcgcgctgttcgacccggaccccgacgccaccggccgctcctacgtcaccgagggcgggttcctggacgacgcggccctgttcgacgcgggcttcttcgggatctccccgcgcgaggcgctggccaccgacccgcagcagcgggtgctgctggagaccgcgtgggagaccttcgagcaggcgggcatcgacccgacctcgctgtccgggcaggacgtgggcgtgttcaccggggtcgccaacggggactacgcgctgaccgtggaccgggtgccggagggcttcgagggctacctgggcatcggcggggcgggcagcatcgcctccgggcgcatctcgtactccctgggtctggagggtccggccgtcacgctggacaccggctgctcgtcgtcgctggtcgcgatgcactgggccgggcacgcgctgcgggcgcgggagtgctcgctggcgctcgcgggcggcgtgatggtgatggcgacgccgggtgggttcgtcgggttctcccggcagcgcgggctggcccgcgacgggcggtgcaagtcgttcggcgacggcgcggacggcacgtcgtggtcggagggcgtgggtctgctgctgctggagcggctgtcggacgcgcgggccaacgggcacgaggtgcttgcggtggtgcgcgggtcggcgatcaaccaggacggggcgtccaacgggctcaccgcgcccaacgggccgtcgcagcagcgggtgatccgcgcggcgctcgacgcggcggggctcgggcacgcggacgtcgacgcggtggaggcgcacggcaccgccacggtgctcggcgacccgatcgaggcgcaggcgctgctgaacacctacgggcggcaccgggacggggcgcagccgctctacctggggtcggtcaagtccaacctcgggcacacccaggcggcggcgggcgtggccggggtgatcaaggcggtgcaggcgatgcgccacggcgtgctgccgcccaccctcaacgtcggcacgcccaccaccaaggtcgactggtcctcgggcgcggtggaggtgctggcggaggcccggccgtggccggagaccgggcgtccgcgccgggtgggcgtgtcgtcgttcggcgtgagcggcaccaacgcgcacgtgatcctggagcaggcacccgagcacgagccagcgccggaggagccgggtgggcgcgggccggtggcggcgggcggcgcgacgccgtggacgctgtccgggcgcacgcccgccgcgctcgccgaccaggcgcggcggctggccgggcacgtgacggccgacctgcgggcggaggacgtcgggttctcgctggccaccaccagggcgcacctggagcaccgggcggtggtggtcggctcggacgggctggcggcgctggccgaaggccgctcgtccgcgctcgtgacgaccggtgaggccggggtcgacgggcgcgtggtgttcgtgttccccggccaaggggcgcagtggatcggcatgggcgcggagctgatcgacgcgtcgccggtattcgccgagcggttgcgcgagtgcgcggaggcgctggaaccgttcgtggacttcgacctgatcgaggtgctgcgcggacgcgggtcgctggagcgggtcgacgtggtgcagcccgcgtcgtgggcggtgatggtgtcgctggcagcgctctggcggtcgctgggcgtggaaccggacgccgttgtcgggcactcgcagggcgagatcgcggcggcggcggtcagcggggcgctcagcctgcccgacgccgcagccgtggtcgcgttgcgcagcaaggcgatcgcccaggacctggccgggctcggcggcatgatgtccgtcgccctgcccgccgacgacgtcgacctgagcgggtatcccggacgcctgtgggtcgccgcgcacaacggccccacctcgaccgtggtggccggtgacgtggacgcgctgcgcgagctccacgcccactacgagggcgccgaggtccgggcccggatcatccccgtcgactacgccagccacaccgggcacgtcgacaccatccgcgagcggctcgccgaggcactggcgcacgtgcggccgagggcgggcacgatcccgtggctgtcgaccgcgaccggcgagtggaccaccggtgaggacgccgacgccgactactggttccgcaacctgcgcggcgcggtgggcttccacaccgccatcaccaccctcgccgagcagggccaccgggtgttcgtggaagtctccagccaccccgtgctcaccaccgccatcgaggccacgctcgaaggaaccggacccaccgccgtcaccggaaccctccgccgcgacgacggcggccccgaccgcctcctcaccagcctcgccaccctgcacgtgcgcggcgtccacgtcgactggaaggccgtgttcgccggcacgggcgcgcgccgcgtcccgctgccgacctacgcgttccagcacgagcgctactggctggaccggggcgcggcggccggtgacgtcacgggcgcgggcctggccgacgcggcgcacccgctgctggccgccgtcgcccagctgcccggcaccggcggggtgctgctgagcgggcggttgtcgcgggcgacgcacccgtggctggccgagcacgtggtgaacgggaccgcgctggtgcccggcacggccctggtggagctggcgctgcgcgcgggcgacgaggtggacgcgcccgtgctgcgcgagctggtgatcacccggccgatgccggtgccggagcggggtttcctgcacgtgcaggtggacgtcgcgggtgcggcggacgacgggtcgcgggcggtgcggatctggtcgcgcgccgaggacgcggcgagcgagacggcccgctggaccgagcacgccaccggctccctcgcccccgacgacgcggccccgcccgcccgcgcgagcggcgcgtggccgcccgagggcgcggcggccgtggacgtggacgacttctacgaccgcctcgcgggcgcgggctacgagtacgggccgctgttccagggcctcaccgccgcgtgggccggggacgggcaggcgtgggccgaggtggtgctgcccggtgaggcgggcgggttcggcgcgcacccggcgctgctggacgcggcgctgcacgtgggcacgttctgcctgcccggcgggccggggtcgcgcacgctgctgccgttcgcgtggacgggcgtgcggctgcacgccaccggcgcgacggcggtgcgggtgcacgcccgcgccaccggcgacgacggcctcgtcgtggagctgcgcgacgcggacggggaaccggtcgtgacggtcgacgcgctcgtgctgcgcgacgccgaccccgccgacgcgcaggccccggacgtcacggcggacgcgttgtggcgggtgcggtgggtcgagcagccgcccgcgcccgcggcgcccggctgggtgctgctgggcgggccgtccgggcacgccgggttcgccgccctgccggtgttcgccgaccctgcggccgtggcggcgctgcccgagggcgaccggcccgcggtggtcgtcgtggacaccaccgcgtgtcgggagccggggcgggacgtgccgggggcggcgcgggcgttcgtggtgcgggcgctggagctgctggtggcgtggctgcgcgaggacgcgctggccgggacccgactggtgctagtcaccagcggcgcggtgccggtgcgcgcggacgccgaggtcaccgaccccgctgccgcggcggtgtggggtctgccgcgctcggcgcagtcggagcacccggaccgggtgtggctgctggacgtcgacgagccgggcgcggcgccgggcgcgctggcctcgccggagccgcagctggccgtccgggcgggcgcggggttcgcgccccggctcgccagggccgaggccgcgcccggcgcgctgccggtcgacgggccggtgctggtcaccggcggcaccggcacgctcggcgcgctcgtggcccggcacctggtcaccgcgcacggcgcgcggaacctgcacctggtgagcaggcgcggcccggacgcgtccggcgctcgagaactcctggacgagctgcgcgggctgggtgccgaggtcgagctgtcggcgtgcgacgtggccgaccgggtggcgctcgccgccctgctggggcgcgtgcgccccgccgccgtggtgcacgcggcgggcgcggtggacgacggcctgctcaccgacctgaccgccgaccggttcgacgccgtgctgcggcccaaggtcgacgcggtcgcccacctggacgacctactcggggacgtgccgctggtggtgttctcctccgcgaccggcaccctcggcacccccggccaggcgaactacgccgcggccaacgcggtcgccgacgcgctcgtgcagcgccgccgcgcgcggggcctgccgggcgtgtcgctggcgtggggcctgtggtcggacaccagcgagctgaccgcgaccatggacgccgccgacgtggcccgcacccgccggggcggggtgctcggcctggacgcggcgcgcggcctggcgctgctcgacgccgcgctcggcgcggacgacgcgctgctcgtgccgatccacctggacaccgccgcgctgcgccggggggccgacccggctccgccgctgctgcgcggcctggtccgccgcgcccggcgcgcggcgggcgcggcccggcaggccgcgctgccgctggtggcgcgactggccggggtggacgcggcggagcggcggcgggcgctggtggagctggtgcgcgccgaggccgccgccgtcctcgggcacggcggcccggacggcatcgggcaggaccagccgttccgggaggtcgggttcgactcgctcacggccgtggagctgcgcaaccggctcggcgcggccacgggtctcgcgctgcccgcgacggtggtgttcgaccacccgacgtccgcgcgggtcgccgagcacctgcgggagctgctgttcggcgcggagacggctcaggcccccgcgcggcgggaggtggtggccgacgacgacccgatcgccgtggtgggcatggcctgccggttccccggcggggtcgccgacgcggacgggctgtggcggctggtcgccgaggagcgcgacggcatcggcccgttcccggacgaccggggctgggacctggcggcgctgttcgacccggaccccgaccacgcgggcacctcgtacgtgcgggaaggcgggttcctcgacggggcgaccgggttcgacgcgccgttcttcggcatctccccgcgcgaggcgctggccatggacccgcagcagcggctgctgctggaggtggcgtgggagacgttcgagcaggcgggcatcaacccgcgctcggcgcacggcaccgacaccggggtgttcgcgggcgtgatctaccacgactacggcgaggcggcgggcgagctgcccgagggcgcggagacctaccgcagcaccggcacgtcgggcagcgtggtgtccggccgcgtcgcctacgcgctgggcctgaccggcccggcgctgaccatcgacacggcctgctcgtcgtcgctggtggcgatccacctgggcgcgcgggcgctgcgggcgggcgagtgctcgatggcgctggtcggcggggtgacggtgatgtcgacgccgggcgggttcgtgagcttctcgcggcagcgcgggctggccccggacgggcggtgcaagtcgttctcggagggcgcggacggcaccgggttcagcgagggcgtcgggctggtgctgctggagcggctgtcggacgcgcgggccaacgggcacgaggtgcttgcggtggtgcgcgggtcggcggtgaaccaggacggggcgtccaacgggctcaccgcgcccaacgggccgtcgcagcagcgggtgatccgcgcggcgctcgacgcggcggggttggggcacgcggacgtggacgcggtggaggcgcacggcacgggcaccaccctcggtgacccgatcgaggcgcaggccgtgctcgccacctacgggcaggaccgcgagcagccgctgtggctgggcacgctcaagtccaacctcgggcacacccaggcggcggcgggcatcggcagcgtgatcaagatgatccaggcgatgcggcacggcgtgctgccgcgcaccctgcacgtcaccgagccgaccacggccgtggactggggcgcgggcgcggtggagctgctgacgcgggcgcgggagtggccggagaccgggcgtccgcgccgggcgggggtgtcgtcgttcggggtgagcggcacgaacgcgcacgtgatcctggagcaggcccccgaaccggtggcggtggaggcggcgccggaggcgggggtgctgccgtgggtgctgtcggcccgcacgcccgaggcgctgcgggagcaggccgaccggctcgtggcgcacctgggcggtgagtcgtcctcggcggccgtggcccggtcgctggtgctgggtcgggcggccctggaggagcgggccgtggtcgtgggcgaccgggcgcgcgccggggaggcgttgcgggcgctggccgaggggcggccctcccccgcgctcgtcaccgggcggaccggggtcgaggggcgcgtggtgttcgtgttccccggtcagggcgcgcagtgggtcggcatggggcgtgcgctgctggacgcctcgccggtgttcgccgaacgcctgcgcgagtgcgcggcggccctgcgcccgtacaccgactgggacctggtcgaggtgatcacctcgggtggcgcgctggacgacgtggacgtcgtgcagcccgcgtcgtgggcggtgatggtgtccctcgcggcgctgtggcgctcgctcggcgtcgaaccggacgcggtgatcgggcactcgcagggcgagatcgccgccgcgaccgtcgcgggctggctcagcctccaggacggcgcgaagatcgtcgcgctgcgcagccagctgatcgacgaggagctgaccgggctgggcggcatgatgtccgtcgccctgcccgccgaggacatcgacctgagcggttacgagggccggttgtgggtcgcgacggtcaacgggccgagcgcgaccgtggtcgccggggacaccggggcactggaggagctgcggcgcggctgcggcgaggcggtccgcacgcgggtgatccccgtggactacgccagccacaccgggcacgtcgacgccatccgcgaccagctcgcccggatgctcgccgacgtcaccccgcggcccggcgagatcccgtggctgtccacggtgaccggcgagtggatcacccccggcgacgacgacgccgactactggttccacaacctccgccgcaccgtccacttcgccgacgggatcaccaccctgctcgacgccgggcaccgggtgttcgtggaggtctcctcgcaccccgtgctggcggcggcggtgcaggagagcgccgaggcggccggggacgcgcgggtcgccgtgaccggcacgctgcgccgcgacgacggcggctgggaccgggtcctgaccggcctggccgagctgcacgtgcgcggcgtggacgtggactggacgcgggtgctgcccgaggcggggcgggcgccgctgccgacgtacgcgttccagcacgagcgctactggccggaacccgcgcgcccggccgccgcgccgggcggtggtgacgacgcgctgtgggcggtgatcgagggtggtgacgcggcggacctggccggggagctggccgtggacgaggacgagctggcgcgggtgctgcccgccctgacctcctggcggcggcgcagccgggcaaggagcgcgctcgacggctggcgctaccgggtcgactgggtcccggtccccacgagcgggtctgggctgcccggcgggcaagcgctgtccggcgggcaggcgctgtccggcgggccgaggtcgtccggcggggcagggctctccggcggtcaggggacgccaggcgggtccgggtcgcccggcggagcggcactgccaggcgggccagggtcgcccggcggagcggcgctgcccggccgggtggccgtggtggtgcccgcgaacgacgagcgggcgcgggcggtcgcgggcgggctggtcgcgcggggtgtggacgtgaccgtcgtggcggcggtcgacgccacccgcgacgggctggcgaaggcgctgcccgaccgccccgacgccgtggtgtcgctgctgtcctgggacgcgggggccgacgagccgggcgcgcccggttcggccacggccgcgctggtgcaggccctggccgaccggggtgccaccgggccgctgtggtgcgcgaccgggggcgcggtgagcgtcgcgggcgaggacgccgaccccgaccaggccgccgtgtgggggttgggcggggtgctggccctggacctgccggaggcgttcggcggactggtcgacctgccgcggcagcccaccgacgccgacctcgacgcgttcgccgccgcgctgaccgcccccggcggcgaggaccagctcgcggtgcgcgacggccgcctgctggcccgccgcctggtccgcgacggggccgacgcgccggagtggacgccgcgcggcgcggtgctggtcaccggcggcaccggcggcctcggcacgcacgtggcccgctggctcgcccgctccggggccgggcacgtcgtgctcgccagccgctccggccccgccgcccccggcgcggccgggctggccgccgaggtggaggcgctgggcgcgcggtgcagcgtggtggccctggacgtggccgaccgggacgcggtggccgccgtgctcgccgacgtcgagcgggacgggccgctgaccgccgtggtgcacgcggcgggcgcgggactggccccgacgccggtggtggagctgaccgccgggcggtacgcggacgtcgcggccggcaaggtcgagggcgcgcgggtgctggacgaggtgctcgccgaccgggcgctggacgcgttcgtgctgttctcctccggcgcggccgtgtggggcagcggcgggcaggccccgtacgcggcggccaacgcgttcctggacgggctggccgcccgcaggcgggcgcgcgggctcgtggccacctcggtggcgtggggcggctggggcggcggcctcggcatgatcggcgacggggacgcggagcggtgggcccggctgggcatccgcacgatggacccggaggcggcgctgcgcggcatggcgctggcggtcggctccgggcgggccgcgagcgtggtggccgacgtcgactgggcccggttcgcccccggctacgccctggcgcgggagcgcccgctgctgcgcgggctgccggaggtggtggcgctgctggccgaaccggacgagcccgccgcggcggtggacgcgcggggcgcgctggcggcccggctgaccgggctggacgcggccgggcaggacgagctgctcgcggacctggtgcgggcgcaggcggcggcggtgctggggttcgccgaccctggcgcggtcgcggcggaccgggcgttcaaggacgccgggttcgactcggtgaccgccgtggagctgcggaaccggctgggcgcggccaccgggctgcggctgccgccgaccgtggtgttcgaccacccgaaacccctggctctggcgcgcgtgctgcgcgccgagctggtcccccagcggggggacggggtgacggcggcgcaggtggcgcaccgggaggacgcgatccggcgggtgctggcgtcggtgccgctggcccggttcgaggagctgggcgtgctcggcgcgctcgtggacctcgtgcccgccgcgccaccggcgggcggcgcggcgacagcggagcgggacgacctggcggacctggcggagctggacctggacggtctggtccgcagggcgatgcgcggcaccaccgccgggaacgactgaggctttgatgcggagcggagagagcatgagcgcgggcacctcgccggagagcgtcgtccaggccctgcggaccacgctggtggacaacgagcggctgcggcgggagaacgagcggctggtcgccgaggccggtgagccggtggcgatcgtgtcgatggcgtgccggctgcccggcggcgtcaccgacccggagtcgctgtgggagctggtgcgcgagggccgggacgccatcgggccgttcccgaccgaccggggctgggacctggggtcgctgttcgacgacgacccggacgcggcgggctcctcgtacgtgcgggagggcgggttcctggcgcgggcgggcgggttcgacgcgccgttcttcggcatctccccgcgcgaggccctggccatggacccgcagcagcggctgctgctggaggtggcgtgggaggccgtggagcgggccgggctcgacccgcgctcgctggagggccgggacgtcgcggtgttcgcgggcggcaacccgcagggctacggcggcggaccgggtgacgccccggagggcctggaggggttcctgggcgtcaacgcctcgtcgtcggtgatctccgggcgggtctcctacaccctgggcctgaccggcccggccgtcaccgtggacacggcgtgctcgtcgtcgctggtggcgatccacctggcggtgcggtcgctgcgctcgcgggagtgctcgatggcgctcgcgggcggggtgaccgtgatggggcagccgaccgcgttcgtggagttctcgcggcagcgcgggctcgccccggacgggcggtgcaagtcgttcggcgacggcgcggacggcacgtcgtgggccgagggcgtcggcgtgctgctgctggagcggctctcggacgcgcggcgcgacgggcacgaggtgctggcggtgatccccggctcggcggtgaaccaggacggggcgtccaatggcctgaccgcgccgaacggcccgtcccaggagcgggtgatcgcggcggccctggccgacgccggtctcggcctggccgacgtggacctgctggaggcgcacggcaccggcaccaggctgggcgacccgatcgaggcgcgggcgctgctcaacacctacggccggggcaggccgcaggaccggccgctgtggctggggtcggtgaagtcgaacctcgggcacgcccagtcggcgtcgggggtggcgggcgtgatcaaggtggtgcaggcgatccggcacggcctgatgccgcgcacgctgcacgccgacgagccgagctcgaacgtggactgggcggcgggggcggtggagctgctggcgcgcgagcgggagtggccggagaccgggcgggcgcggcggggcgcggtgtcgtcgttcggggtgagcggcacgaacgcgcacgtgatcgtggagcaggcgcccgaggaggccgccgccggggtcgcggcggcggggcggcccgcgcccaggtcggcgggcgggcaggacgccgggatcgcggcggtgaccgggcaggccgcccccgccgctggccccgccaccgccgaacccgccgcgtcggccgtcgaggacgggaccggcgtcgcccccggcccggtcgcgaccggcggggtcgtgccgtgggcgctgtccgggcggaccgccgccgcgctggccgcccaggcggcccggttgcgcgcgcacctcgccgcgcacccggcggcccgcccggtggacgtggcctggtcgctggccacgacccgctcggtgctggagcaccgggccgtcgtgcccgccgcctcgctcgacgaggccctggcggggttggacgcgctcgcctcgggccgcgcggaccggtcggtggtcgtcggcgaggcggcgcccggccgggtggcggtgctgttcaccgggcagggcagtcagcgggccggtgcggggcgcgagctgcgggagcggttcccggtgttcgcgcgggcgttcgacgccgcgtgcgccgccgtgggcgagctgcccaccggcgacggcggcgcgatcgggctcgccgaggtggcgctggccgaccccggcacgcccgccgccgcgctgctcgaccggaccgcgttcacccagcccgccctgttcgcgctggaggtcgcgctgttccggctggtccagtcgtggggcgtgcgcccggcggcgctggccgggcactcggtcggcgagatcgccgccgcgcacgtggccggggtgctctccctcgccgacgccgccgcgctggtgcgcgccaggggcgggctgatgcaggagctgcccgagggcggcgcgatggtggcggtggaggcggccgaggacgaggtcgtgccgctgctcggggacggggtgtcgctggccgccgtcaacggcccgacctcggtggtgctctccggcgacgaggaggccgtcaccgccgtcgccgcgaggctggcgcagcggggcaggcgcaccaagaggctcgccgtctcgcacgccttccactcgcaccgcgtggacccggcgctggccgccttccgcgccgtggccgaggagctcgcctacgccgcccccacgatcccgatcgtctccaccctcaccggccgccccgtcacccccgacgagctgcgctcccccgactactgggtgcggcacgcgcgcggcgccgtccggttcctggacgccgtgcgggcgctgggggacgcgggcgcgcgcacgttcctggagctgggcccggagggcgtgctcacggcggcgggcgcggactgcctgccggacgcggtgttcgcggcgacgctgcgcgccgacgtgcccgaggcgcgggccgtgctcgccggggtcgcgggcctgcacgtgcgcggcgcgacagtcgactggggttcgctgttcacgggcgcggacgcgcggcgcgtcccgctgcccacctacgcgttccagcacgaggaccactggctggtgcgccgctccaccgccgccgacgtgggcgcggtcggcctgcgcgaggccgggcacccgctgctgggcgcggtcgtcgcgctgccggagagcggcggggtgcagctgagcggtcggttgtcggtggcggcgcagccgtggctggccgagcacgtcgtctccggcacggcgctggtgccgggcgcggcgctggtggagctggcggtgcgggcgggcgacgagaccggcacgcccgtgctggaggagctggtgatcggccgcccgatgccgctgccggacggcggcgcgctgagcgtgcaggtcgtcgtcggcccggacgagggcgggcgccggtcggtgcgcgtgtactcccgcgcggacggggcggtggactgggtcgagcacgcggcgggggcgctgaccgcgccggaggccgcgccgaccgccgacgcgggcccgtggccgccggagaacgccgaacccgtggacacgcggggcttctacgacaccctcgcggagggcggctacgcctacggcccgctgttccggggcctgacctcggcgtggcgcggcgagggcgaggcgtgggcggaggtggcgctgcccggtgacgcgaccgggttcggcatccacccggccctgctcgacgccgcgctgcacaccgcgcacttctgcctgcccaccgggaccgagcggcgggccgggctgctgccgttcgcctggaccggcgtgcggctgcacgcgggcggcgcgacgaccgcgcgggtgcacgcccgcgccaccggcgacgacggcgtgaccgtgcgcctgctcgacggtgccggtcagccggtcgcggacgtggccgccctgaccttccgcgccgcagccgacaccccgtccgccgaggtcccggacgcgctgtgggcggtggagtggaccgagcacccgctgcccgcggacgggaccacccccgcgggcgggaccaccacggccgtggtggtcgtggacacccggagcgtcgacgcccccgacgacggccccgcccgcgcccgcgcgctgaccgcccacgtcctcgccgagctgcagcggcacgccgacgacgaccggccggtcgtcgtggtcacctcaggcgcggtcgccgtgcgcgtcgacggcgaggtcaccgaccccgcgtccaccgccgtgtgggggctggtgcgggccgcgcaggtcgagcagcccgaccgggtccggctggtcgacgtcgagccgggggccgacccggtgctcacctcgcccgagccgcaggtggcgctgcgcggcgggaccgcgcacgtgcccaggctggtccgcgcccgccgcgccctcccggcgccgaccgcgacgtcgtggcggctgggctccgaccgccccggcacgctggactccctcgccctgctcccggacgactccggcacggccccgctcgcccccggcgaggtgcggatcgcggtccgcgcggcgggcctgaacttccgcgacgtgctggtcgcgctggggatgtaccccggtcgcgcggtgatcggcgcggagggcgcgggtgtggtcgtggaggtcggccccggccccgacgacaccgacgccggcgacaccggccccggcgacaccggctcgggcggcctggccgtgggcgaccgggtgatgggcctgttccccggcgcgttcggcccgctggccgtggccgaccaccgaatggtgacccggatgccggacggctggtcgttcaccaccggcgccggcgtgcccatcgcgttcctgaccgccctctacgggctgcgcgacctcggcgggctcaccgcgggcgagaccgtgctggtgcacgcggcggcgggcggggtcggcatggccgccgtgcagctcgcgcgggcgttcggcgctcgggtgctgggcaccgcgcacccggccaagcacgcggccgtgacccgcctgggcgtccccgagtcccacctgtcctccagccgcgacaccgcctacgccgacctgttcggcccggtggacgtggtgctgaactcgctcaccggcgagcacgtggacgcctcgctggggctgctgcgcgcgggcggccggttcctggagatgggcaagaccgacctgcgcgacgccgacgaggtcgcgaaggcgcaccccggcgtcgcctaccgcccgttcgacctgggcggcgaggcgcccgccgagcgcgtcgcggagctgctggccgagctggtcgcgctgttcgaggcgggccgcatccacccgctgcccaccgcggcctgggagatcacccgcgcgccggaggcgttcggctggatgagccgggccgggcacgtgggcaagatcgtgctgaccctcccccgccgccccgacccggacggcacggtgctggtcaccggcggcaccggctcgctcggcgcggtcgcggcccggcacctggtcaccgcgcacggagcccgccacctgctgctcgcctcccgacgcggcgagcaggcccccggcgcggcggagctgaccgacgggctgcgcgggctgggcgcggacgtgcgggtcgcggcgtgcgacgtcgccgaccgggacgcgctcgccgcgctgctcgccacgatccccgccgcgcacccgctcaccgccgtcgtgcacacggcgggcgtgctcgacgacggcgtgctcgccgcgcagacccccgagcgcctggacgcggtgttccgccccaaggtcgacgccgtcgcgaacctgcacgagctgaccggcgacccggccctgttcgcggtgtactcctcggcctccggcgtgctcggcggcgcgggccagaccaactacgccgccgcgaacgcctggctcgacggcctcgcccacgtccggcgcgcggcgggcctgcccgcgacctcgctggcctggggcctgtgggcgcaggacggcggcatgacgggcggcctggcgggcggaccggccgggccgggcgggcgggcccgccggggagccgtcgcgccgctgtccaccaccgagggcatggcgctgttcgacgcggccgtcgcgtcgggccgcccgctcctggccccgatcaggctcgaccccgccgcgctcaccgccgacggcgcgcagccgcccgcgctgctgcgcggcctggcccgccccacccgccgcaccgccgtcgcggccaccaccgacgacggcctcgcgggcaggctcgccgcgctcgacggccccggcaggcagcggctgctcgtggagctggtgcgggagcaggccgccgccgtgctgggcttcgcgaccccggacgccgtgtcgccgggccgggcgttccgggacctgggcttcgactcgctgacggccgtggagctgcgcaaccgcctctccgccgccaccggcctgccactgcccgccaccaccgtgttcgaccacccgaccccgctggacgcggcggcccacctgctcgacgcgctgggcgtcgcccccgcgcccgccccggccaccccggtcgtgacggccgcgcgggacgacgacccgatcgcggtcgtcgccatgggctgccgcctgcccggcggcgtgtcctccccggaggacctgtggcggctgctcgacggcggcgtcgacgccatcggcccgttcccggacgaccggggctgggacctggggtcgctgttcgacgacgaccccgacgcggtcggcaagtcctacgtgcgcgagggcgggttcctggcgggcgcgggcgggttcgacgccgcgttcttcggcatctccccccgcgaggcgctcgccatggacccgcagcagcggctgctgctggaggtggcctgggagaccgtcgagcgggccgggatcgacccgacctcgttgcgcggcgcggacgtcggcgtgttcgccggggcgggcgcgcagaactacggcagcggccccggcccggtgcccgagggcctggagggctacctgggcgtgggcggcgcgacgagcgtggtgtccggccgcgtctcctacacgctcggcctcaccgggcccgcgctgacgatcgacaccgcgtgctcctcgtcgctggtggcgatccacctggcggtgcggtcgctgcgctcgggcgagtgctcgatggccctggcgggtggggtcgcggtgatgggcgagcccgcggcgttcgtggagttctcccggcagcgcgggctcgccccggacgggcggtgcaagtcgttcggcgcggaggcggacggcacgacgtgggccgagggcgcgggactggtgctgctggagcggttgtcggtggcgcgggcgcgcgggcacgaggtgctggcggtgctgcgcgggtcggcggtcaaccaggacggggcgtccaacggcctgaccgcgccgaacggcccgtcgcaggagcgggtgatccgggcggccctggccgacgcggggatcaccccggacgcggtggacgcggtggaggcgcacggcaccggcaccaccctcggtgacccgatcgaggcgcaggccgtgctggcgacctacgggcaggaccgcgagcagccgctgtggctggggtcgctgaagtcgaacatcgggcacgcgcaggcggcggcgggcgtcgcgagcgtgatcaagtccgtgctggcgctgggccggggcgtgctgccccgctccctgcacgccagcaccccgaccccgcaggtcgactggtcctcgggggcggtggagctgctggcgcgggcgcgggagtggccggagaccgggcgtccgcgccggatcggggtgtcctcgttcggggtgagcggcaccaacgcgcacgtggtcctggagcaggcccccgagccggaacccgcgcgggaggcggaacccgcgcgggagtccgcgccagggccggagtccgttccgccgctgaccggggccacgccgtggctgctgtccgcccgctcccccgaggcgctggcggaccaggccgcccggctggtggacgccgtgcccgccgagtggcgggcctccgacgtgggctggtcgctggccaccacgcgggccccgctggagcagcgggccgtggtcgtggcgcgggacaccgcgcgcgggctcgccgccgcgtccgcgctggccgccggacgccccgacccgcacgtggtcaccgggaccgccgacgtggacggcaggaccgccttcgtcttccccggccagggcgcgcagtgggcgggcatggggcgggaactcctggacgcctcgccggtgttcgccgaacgcctgcgcgagtgcgcggcggccctgcgcccgtacaccgactgggacctggtcgaggtgatcacctcgggtggcgcgctggaggacgtggacgtcgtgcagcccaccagctgggcgatcatggtgtcgctggccgcgctgtggcgctcgctcggcgtccacccggacgcggtgatcgggcactcgcagggcgagatcgccgccgccaccgtcgcgggctggctcagcctccaggacggcgcgaagatcgtcgcgctgcgcagccagctgatcgacgagcacctgaccgggctcggcggcatgatgtccgtcgccctgcccgccgaggacatcgacctgaccggctaccagggccggttgtgggtggccgcccacaacggccccaccgcgaccgtggtcgccggggacgccgacgccctggcggagctgcgggacgcgctggagggcgaggcccgcacccgcgtgatccccgtcgactacgccagccacaccggccacgtcgacgccatccgcgaccagctcgcccggatgctcgccgacgtcaccccgcggcccggcgagatcccgtggctgtccacggtgaccggcgagtggatcacccccggcgacgacgacgccgactactggttccacaacctccgccgcaccgtccacttcgccgacgggatcaccaccctgctcgacgccgggcaccgggccttcgtcgaggtctccacgcaccccgtgctcaccccggccgtgcaggaggccgccgaggcgaacccggcgctgcgcaccgtcgccgtgggcaccctgcgccgcgcggacggcggcgcggagcgggtggtggcgggcctggccgagctgctggcgcgcggggtggccgtggacccggcggcggtgttccccggtgcgaggcgggtcgcgctgccgacgttcgcgttccggcacgagacgttctggctctcgcgggcgctgcccgacgcgcggccggtgccgcagggcgggcacccgctggccccggtggtggtgagcgatccgggcacgggcggggtgatcctgtccggccggatctccgcggccacccacccgtggctgctcgaccacgccgtcgcgggcgcggtgctgctgcccggcgcggcgctggccgagctggcggtgcgggccggcgacgagaccgggacgcccaccctggaggagctggtgatcggcaggccggtggtgctgcccgaggacggggagctgcggctccaggtggtcgtgggcgccgaggacggggcgcgccgcgaggtgcgcgcctactcccgcgccgacgacgccgcgccgtggaccgagcacgcgagcggcacgctgtcggcgaagtcctcgctgcccgccgacgtcccggccgccccgtggccgcccgcgggcgcggagccgatcgcgctggacgggttctacgaggccatggcaggggccggttacgggtacgggcccgcgttccgggggctgcgcgcggcctggcgcgacggggacgacgtggtcgccgaggtggccgtgccgcgggcgcaggagcaggtggcgggccggttcggcatccacccggcgctgctggacgccgccctgcacgccgggaacttctgcttccccgcgcaggacggcgagcgggccacgatgctgccgttcagctgggacgacgtgcggttgcacgccaccggcgcgacgtcggtgcgggtgcgggcccgcgcggtgggcggccctggcgcgcccgcgctgaccgtggcgatcaccgacccgagcggggtgccggtggccggggtgggcgcgctcgggatgcgcgcggtcagccccgagcagctgggcgcgccgggcgtcggcggtgacgcgctgcgggtgctggagtgggccgaggtggcggtcgaggcggcggaccggtgggccgtgctgggctccgagcggcacccggacgtggacgcctacgcggccgacccggaccggccgggggcgctgctggtggacgtgggcgcctggctgggcggcgacgacgccgtggcccgcgcgcacgcgctgaccagcgcggcgctggagctggtgcgggactgggcgacccgcggggacctgggcggtgagcggctggtgctggtcacgaccggggccgaggacgtgcgcgacaccgcgccccgcgacccggcgcaggccgccgtgtggggcctggcgcgctcggcccgctcggagcacccggaccggttcgcgctggtcgacgcggacgaccggtccccggcgacgctcgccctggcggccgggtcggcgttcccggaggtggtcctgcgcggcgagcgggcgcacgcgccgaggctggcgcgggccgtccccggcaggccggtggcgctggacccggacggcacggccctgatcaccggcggcaccggcgccctgggcgcgctcgccgcccggcacctggtgaccgcgcacggcgtgcggcgcctgctgctcaccggccgccgggggccggacgcccccggcgcggcggagctggccgaggagctgcgcgggctgggcgcggacgtgcgggtggaggcgtgcgacgtcgccgaccgggacgcgctcgccgcgctgctcgcgtcgatccccgccgggcgcccgctcaccgccgtcgtgcacgcggcgggcgcgctcgacgacgccccggtgaccgacctgaccccggagcggctgtccgccgtgctggccccgaaggtcgacgcgctggccaacctggacgagctggtcggggacgggcccgcggtgttcgcggtctactcctcggcgtccggggtgctcggcacggccgggcaggcggcgtacgcggcggccaacaccttcgcggacgcgctggtgcgccgacgccgggccgagggccgggcgggcgtgtcgctggcgtggggcctgtgggcaggcgccagcgagctgaccggcgacctggccggtgaccggctcgcccgcacccgccggggcgggctggtgccgctgaccgccgccgagggcatggcgctgttcgacgcgggcgcggtcaccacgggcggcccggcgctggtcgtgccgctgccgctggacctggcggcgctgcgcgcctccgcgcgcgacgaggcggtgcccgcgctgctgcgcgcgctcgtccccgccgcgcggcgctcgctctcccccgccaccgggcaggccgcgcccccggccgggttgcgggcgcgcctggccgggctgtcgggcgacgagcaggaggccgtgctcaccgagctggtccgcgacctggccgccgccgtgctcgggcacggcgagaagggcgcggtgggcccggacgacgcgttcttcgagatcggcttcgactccatgacggccgtgcagctgcggaaccggctgaacaccgccaccgggctgcgcctgcccgccgcgctgctgttcgaccagccgacgcccgcgatcgccgccgaggcgctgcgcgagcgactggccgccgagcaatcgggctcagggcaatcgggcgcagggcagccgggcgcagggcattcaggcgcagggcagtcgagcgcagggcgatcaggcgcagggcagtccaccgacccgaccgacgagaggtgagcaccagcatgatcgacgtggccgagtacctgcggcgcatcggcgtggagggcggcgtgccgagcccgacgctggagtcgttgcgggcgctgcacaagcggcacctgatgtccgtgccctacgacaacggcggcgcggccgaccggttgccgccgaaccgggggctcgcggagatcccgctgccccgtgtgttcgcgcacgtggtgaccggccgcaacggcggggtctgctacgagctcaaccggctcttccacgccctgctcaccgcgctgggctacgaggtgctgatggtcgcggcggcgatccggctggccgacgaccggttcgggccggacgaggagcactcgttcaacctggtgcgcctggacgggcggacctggctggtggacgtggggttcgtcggcccgtcctacctggagccgctggagctgtcggcggtcgagcaggagcagtacggctgcgcctaccgggtcgtggagcgcggggacgcgcacgtggtggagcgcaggcccagggacggggcgtggcaggcggtgtaccggttccggccggggcgggcggaccgggacggctgggaggcggtgcggttggacgggctggacgactacgcgcgggactcggtgctggcgggcaccacgttccggggtcgggcggcggagaacgggcagcacgtgctgatcggccgccgctacttcaccgtgctggacggggtggagacgacgcgggtgctcgtgaagaaggacgagttcgcccgcgtcaccgagtcgatcatgatcggggggtgagcgcgtggcgggcgaggtcgagcacgacgtggtggtcgtcggctacgggccggtggggcagctgctgtcggtgctgctggcgcagcgcggctggcgggtgctggtgctggagcgctggccgacgccgttccggctgccgcgcgcggtcgggttcgacagcgaggcgacccgcgtgctggcctcggccgggctcgggcccgcgctggccgagttcggggagcccgcgggcgactacgagtggcgcaccgcgtccggggagacgctgatcgcgttcaccgtgcgggaggaggggcactgcggctggcccgaggcgacctcggcctaccagcccgcgctggaggacgcgctgatcgcgcgcggcgaggcgctgccgggggttcaggtgcggcgcggctgggaggtgaccgggctgaccgaccggggcgaccacgtgcgggtggtggccaccgaccccggcggggcgcgcgtgaggctgacggcgcggttcgcggtcggctgcgacggggcgaacagcgtggtgcgggcccgcaccggcaccgacgtgaccgacctggacttctcgcacgactggctggtgtgcgacgtgcggctgcacgaccggcgcccggtgacgccgaacaacctccaggtgtgcgacccggccaggccacgcaccgcggtgtcggcggggccagggcaccggcggtacgagttcatgcgggtgcccggcgacgacccggagcggttcggcacgccggagagggcgtgggagctgctggcgctgttcggcgtcgggcgcggcgacggggtgctggaccggctggccgtgtacacgttccaggcgcggtgggcgcggcggtggcgggcgggccggatgctgctggccggggacgccgcgcacctgatgccgccgttcgccgggcagggcatgacctccgggttccgggacgcggcgaacctggcgtggaagctggacctggtgctgcgcggcgaggccgggtcggcgctgctggacagctacacgctggagcgcgccgagcacgtgcggcacgccgtgacgatctcggtgggcctggggcgggtggtgtgcgtggccgacccggcggtggctgcggaccgggacgcggcgatgctggcggcgcgcgagcgcgagctgacaccgggcgcgtcggcccggtcggtgctcaagcccctggaggacggggtgctgcaccgggacggcgacggcgccctcgcgccgcacgcgggggccgtgggcccgcagtggcgggtggggcgcggcgggcgggtcgggctgttcgacgacgtggtggggaccgggttcgcgctgctcaccaggggcgggctggtggcggggccggaggtgcgggcgcggctggacgggctgggcgcgcgctacgcgcacctggtgcccgccggggcggcggcggacgggccggacgacgtggtcgacgtgagcgggaactacctgacgtggctggaggagctggacgcggcggcggtgctgctgcgaccggacttctacgtgttcggcgcggccggggacgcggcggggttggccgggctggtggcggacctgcgcgcgcggttggggtgacgccccgcaggccccggcacgtgccgcgccggggcctgctcgcgcgtcacgtccggtcgtcggcgaggtgggccaggcaccagtcgagcacctgcgagggcttgcggaccaccgcgtccgggttcgccgccagcagctccgcctcgtccgtctcgccccacagcgcggccagcgccgggtagcccgcggcgcgggcgctggccaggtccgtcagggcgtcgcccaccatcaccacccggtcggccgggacgtcgagcaggccggtggccagcaggagcatgtccggcgcgggcttggggttcgcgacctcgtcggagccgatgatatggtcgaacagccccgccatgccgagggtggtcagcagcgaccgggcgcgcggcccgctcttgccggtgaccacggcggtgccgaagccgtgctgccgcaggtccgccagcagctccggcgcgccctcgaacacctccacctcacccgccagccggtagctctcgcggacgaacggaccctccatctccagcggcaggtccatgatccgcatgatgtccgggaagtaccgccccaggtgccggttgtactcctcgaacggcgcgggcccgtcgccgacgacctcggcgtaggcgatctcgaacgcctgccgcatgacggcgaagctgttgaccagcaccccgtcgaggtcgaacagcacggcccggtcgtaggtcgcgccggggacgtgccggtggggcgcgggggtcggcggggcgagggggcgcggggccgcgggcgccggaaccgcggtcgcggcccgctcgtccccggctcgggcccgcacgactcgggggttggtctgtccggtggtcatcacggggctcccgtcgggacgaggtcgaccggcgcgtgtcgtcgttcggcgcaccgcacggtgtcggcggcgcggtagacccgttcgatcgcgcccgcgatccagcgcgccccggacgccgcctcgccgcgcgtggcggggtcggccaggcgggtgggcaggctcgcgagctgggcgtcgtactcggcgcccaccggctcggcgggcagctcgaccggggtggtgcggccgtcggtggtgagcaggagccgcgacgggccctcgcggttcgggctgaagccgaaggtgcagcgcagctccgccgtgccgccgctgccctccacgcgcacgaccgtggtgtccagcgcctggtgcgaggcccaggccgcgcgcaccccgatcgagatccccgaccgggtgacgaggaaccccctggcggtgtcctccacgtcgccgaccaccgggtccaccggcgccccgtcgccgcgccaggcggcccggaacgcgtggtcgttgacgaagtccgcggacaccgcgccggtgacgtgctccagctccgcgccctccgggtcgccggtggcgccgcgcagcagcacgcgggcggtgtcgagcaggtgccagccgaggtcgaccagcgcgccgccgccggagcgggtgcggttggtgaaccagccgccccggtccgggatgcccttggaccgcacccaggacacgtcgacgtgccgcagcgcgcccagcgacgcggccacctggcgcagcgcccgcacgtcggcccggtgccgggcggcgctcccgcccagcagcaccgcgccaccggcctgctcggcggcggtgagcgcggcggcctcggcggaccccaggcacagcggcttctccaggaacaccgggacgccccgccgcagcagaccggacgcgaccggcgcgtgcaggtggttcggcacggcgaccacggccaggtcgacctcgtcgcggcgcaggtcctccacccgctccagcgcggtgatcccgcgggagccgggcacggcggcgcgggcctgcgcggacggctcgaccacggcgacgacccggaaggcggggctgcccagcaaccggggcagccacacctcccgcgccacccacccgagcccgaccaccgcgacccgcaccggaccaccgctcccggccctcggcccgtcgctcacaccaccacccccgctccccgcgcccgccaccccgcgctcacgcgcccgcgaccacgtcggccacgacggcggcaagccggtgcagctgctcctcggtgcccagcagcacccggtggtgcagccacacgcagtcgcgggtgatctcctccgacaccgggcaccgggcggccagctcctcggtggtcaggtcgggcgcgccggtctcccagaacgcctgggtgcggtagaccgcccggaacgccatgaacgccgggatcccgcgccgcaccagctcgtccaccaccgcgttgcgccgctcctcggtgacgccgggcatccggaacatcgccatgtagctcgggttgcggtcgctgcgcgggtcgacggtctgcggcacgacgccgtcgatccccgccagcagcgcggacagcaccggccagcgggcctgcctggtcgcgatctgcgagtccagcctgccgagctgggcgcgcagcacggcggcggagaactcgttcatccggaagttcgagcccgaggtgaggtggaagtagccgcggtcgcccttgggcctgccgcagctgtgcaggacgaacgccttctcccactgggcctcgtcctcgaacagcacggccccgccctcgcccgccgtcatcagcttgccgttctggaagctgaacgtggcgatcgacccgagctcgccgacccgcttgccgcgccagtgggcgccgtgggcgtgggcggcgtcctgcaggaccggcacgccggtgctcgtggacagcttgtccagccggtccatgtcggcgaactggcccgccatgtgcacgggcatgatcgccgaggtgcgggaggtgacggcggcctcggcggcggcgacgtccaggcagtaggtgtcggggtcgacgtccacgggcacggcgaccgcgccgaggcgctgcacggcctgcgaggacgagatgaaggtgaaggcgggcacgatcacctcggcgccggggcccacgtcgagcacctggagcgccagctccagcgcgtgcgtcccgttggtgacggcgagcgcgtggcccgcgccgtggtactcggcgaactcgcgctcgaactcgtcgacctcgctgccgccgacccgccaccactggccctggtccagcgcgcgcagcagggccgtgcgctcggcgtcgtcgtgctgcggccaggccgggaactcgatgcctgcgtccggagaattgctcatgagcccctgtcccgtcgttcgcggaaatggcgcgggggaattcgccgcggcctgctttcggaattcgacgctaccgattccgcagatcccgaccaacccccttgacctccccctaatcccccctgttcccaggccatcaccgcagcacgcgggcacagcggcacagccgtgcgcacaatgggggcgaacgggaaccggggcgtccgcgcgccccggcggcgctttcggggaaaggtgtcaggcgtgggcgagctgctgctggtgaacgggccgaacctcggcatcctggggcgccgcgaggtgtcggtgtacgggaccgacacgctcgcggacgtcgagaaggcggtcggcgaggaggtcgccgggcgcggctggtcggtccgctcggtgcagcgcaacggcgagggccagctcgtggacgagatcgaggcgtcctacgacacggtgggcgcgatcgtgaaccccggcgcgctgatgatggcgggctggagcctgcgggacgcgctggcgaactacccgcgcccgtggatcgaggtgcacctgtcgaacgtgtgggcgcgcgagagcttccggcacgagtcggtgctggcgccgctggcgagcggtctcatcgcgggcctgggcgcgcgcggctaccggttggccgcccgcgcgctgctggacctggtggactgaccgccgtcgcgcgcgagcccggccgcgtgcacggccccgcgcagcgaggacaggccgccgagcagcgcgggccgcacgggcgcggtcgggtggccgggccgggcgagtgcggcgcagcggtcggcgaccgcgtcgacgtatccgggcacggcggcggcgaacccgcccccgaccacgcacagcgagggccgcgccagctcgccgacgccgacgagcgcggcggccagcgcccgggccccccgctcgaccgcggcccgcgcccacccgcgcccgtcgccgagcgcccgcaccaggtcctccccggtgaccggcgcgccgccgagcctggcggcctcggcgagcacggccggtccggacgcgaacgcctgcacgcacccggcccgcccgcacgggcagggcggcccgtcgagcgccaccacgacgtgccccagctcgcaggacccgcgctcgggtccggggaacggcaggccaccggacacgacgcccccgccgacgccggtccccacgcccgcgtagaccaggtcggcgcacccgtgggcacgggcctcggcgagcgcggcgaggtcgccgtcgtccgcgacgagcaccggggcggccagcccgccgaggaaccccgcgaggtcgacgccgacccaccccggacggctgggccaggccgtgacgaccccgccgtcgacggtgccggggaacgcgatcccgaccccgtcgagcggcgccccggcccgcccggcgaggtcggcgacggcgcggccgagcaggtcgaggtcggcgcgcggatcaccgtccccaggccaccggaagccctcccgcagcaccagcggcccgcgttcgagccgcagcgccaccttggtcccgccgacgtccaccccgagcagcgccccgctcacaccccgacctcccgccgtccgcacccctcgccgcaccaccacccgcgcgggccgccgcccacgacgccgcccgccacaccgatacccgcgcggtcctcgctcacgacgccgccacaccaccaccgcacgggcctgcggtcacgacgccgccacacctccacccagcgcaccaccacccgcgcgggccgccgctcacgaggccaccgctcacgacgccgccgcccgccaggccgccaccgcctcggcccgccgggcgtcgccgctctccaccagcgcgaaccggatcgcccgcgtggccgcgttcgccgcctgcagcgcctcgtgcgcgccgggctcggggtcgctgcgcccggtcgccacctcggtgatgctgcgcgccgcctccaggcacgccaggcaggccgcgacgtacgcgtccgccccgcctcccgcgccgcccagcttctccagcagccgcgtcacgtcggcgtgcacctcgcgcacggcgtcctccacccggcccgcgccgggtggtgtgccggggatgggggtcaccgctgcctcccggtgcctgacgcggacccggtcagccgagggccgggatgagttcgacgaaccgaccctgccacaaccggcgcagctcgcgcgtcaactcctcggacggcgcgccgccgaccagctccgccagggtcgtcacgccgtccacccggctgagcagcgcgtgcgcctgcgcggtggtggcggtgacgggcccgtggtcgtactccagcgacacctcgtgcaccggctcgcccgccgccgcgctgcccggcgcgggcgcggtgcgcacggccaggcgggtcaccgggcgcagcctgggcaccagcgcgcccaggtcctgctcggggcgggcccgcaccaggaagccctccacgaccaggccgtccaggtcggtggtcaggaagctggtgagcacgtcgtccaggctctgcgcgatcggctcggcgttgttgttgaacgaggtgttgagcagcaccggggtgccggtcagctcgccgaaccgcgccaccagccggtggaagcgctctcccgactcgggcgtgacgacctgcacgcgcgcgctgccgtccacgtgcgtgaccgcgcccagctcggcccgcctggcgggcagcaccggcaccacgaacgacatgaactcgtggtgccccagcgccccggacaggtcgaaccagtcccgcgcggcctcggcggtgaccacgggcgcgaacggccggaagctctcccgcttcttcaccatggcgttgatccgcgtctggttctccgcgggccgggcgtcggcgatgatgctgcggtgcccgagcgcgcgcggcccgaactcggagcggccgtgcgcccagcccagcacctcgccgtcggcgagcagcttcgccgcggtctccaccgggtccaccagcggcgtcacctccaccaccggcgaccagtccgcgagccgcgcggcgacctgctcgtccgtgccgaggtccggcccgagcgccgccgacaccagccgcgccgacggccgctccagcacgcccagcgcggcggctgcggcgtacgcggcgccctcgccggcgcccgcgtcgtgcgaggcggggtggatgaacacctcgtcgaacagcccggccttgaggatgcggccgttgagcgtggagttgtgggcgacgccgccgccgaacgcgagcgtgcgcagcccggtgacctcggcccagtgcccgagcacgtgcagcgcgatcttctcggtcgcctcctgcagcgcggcggcgaagtcccggtgcgcctggctgaacggctcgtccttgcggcgcggccggaacccggcggcgaccagcgcgggggtgaccaggttcggcaccttggtgttgccgatcaggtcgtactcgcccttgtcgcgcagcgcgtgcagcccggagaagacctcgcggtaggtcgacgggtcgccggacggggcgagccccatgaccttgtactcgtcgccgaagccgtagccgagcaggaacgtggcgttcaggtacagcccgccgagggacttctccaccgggtagtcgtgcagcttctccaggtgcgcgccacgcgcgtggtagaccgtgccggagttgtcctcgccccggccgtcgaagatcaccaccagcgcctcgtccgcgcccgagtgcaggtaggacgagtaggcgtgcgcctcgtggtgcggaacgtagacgagcttgtcgtcgggcagctcccagccgaggtcctcgcgcagccgctgcttgatcagctcgcgggagaaccgcagcggcaccctcgggtgctcggtgtagacgtggttgagcaccaggtcgaggtggtcctcggggaagtagtagccgaccgcgtcgacgtcgtccacggtcgcgcccgcgagcgccaggcactcgcggatggcggtggacgggaacttggtcgtcttcttgacgcggttgagccgttcctcctccacggcggccacgagctcgccgtcgcgcaccagggacgctgccgcgtcgtggaagaacagctccgacatcgacgggacgaggtcggtctcggcgggcgagaagttcccgttgatcccgagcacgagcatgtggcatcaccttgatccgggaggcgagggctggggcgcggagggggtcgccgtcaggcgcggggcgcgacggcggcgaggtcgggcgaggtcagccgcagcgcggtgggcgcgggcttcgcggtcggcacgaggtggaagcgctccacgccctcctcggtgggcgcgggcagctcggcggcgcaggcgcagtcggcgggggcgaacccggcgaaccggtaggcgatctccatcatccggttgcggtcggtgcgccggaagtcggcgaccaggtgcgcgcccgcgcgcgccgcctggtcggtgagccaggtcagcagcgtcgacccggcgccgagcgagacgacgcggcacgaggtggccagcagcttgaggtgccacacccgccggcgccgctccagcagcacgatgccgaccgcgccgtgcggcccgaaccggtcggacatggccacgaccagcacctcgtgcgcggggtcggcgagcaggccgcgcagcgcccggtcgtcgtaatgcacgccggtggcgttcatctggcttgtgcgcagggtcagctcctcgacgcgggtcaggtccgcctcgcccgcgcgcgcgacgaccacctccaggtccagggtgcgcaggaactcctcgtcgggcccggtgaagccctcgcggctggcgtcgcgctcgaagcctgcgcggtacatcagccgccgctgccgcgagtcggcggtgaccacggcggggctgaactcggggcgctcgggcagggaggcgacgtcggcctcggtgtacaggcgcacctcgggcagggcgcgggccacctcggcgcgctcgacggggctgtcgtcgacgaacgcgatcgtgcggtgggcgaagccgagccggtcggcgatggcgcgcaccgacgccgacttggcgccccagccgatctgcggcagcacgaagtagtcggcaaggcccaggcgttcgagcacgggccaggcgtggtcgtggtcgttgcggctggccacggactgcaggacgccgcgcccgtcgagcgcggtgatcacctcgcggacccgctcgaacgggacgacgtcggcgtcctccaggagggtgccgcgccacagggtgttgtccaggtcccagaccaggcacttgaccgtcggtgcgggggtctcggtcacggctgctgctccctgagcggagttcggctgcgctggtcaagtccgcgcggggcccggctgcgcacgtgctgggcgagcacgagctggcagatctcggaggtgccctcgatgatctccatgagcttcgcgtcccggtgcgcccgcgccaccacgtgcccgtcgctggcgcccgcggagccgagcagctgcaccgcgcgcccggacccggcggcggcctcgcgcgaggccaggtacttggcctgcaccgccgccaccgccaggtccggcgagttcgcgtcccacagcgcgctggcgtgctcgctcgcgcgggcggcgacctgctcgccgacgtgcaactcggccaggtgccgggcgacgagctggtggtcggccagcacgccgccgccctgctcgcgggtcgtggtgtgctcgacggcggcggccaggcaggcgcgcaggatgccgacgcagccccacgccaccgacacccgcccgtaggtcagcgcggcggtgaccaccagcggcagcggcagcccggtgccgcccaggacgtcggcggcgggcacccgcaccccgtccagggtgatcccggagtgcccggcggcccggcacccgctggggttcggcaccctctccacccgcacgccgggcgcgtcggcgggcacgacgaccgcgctcgccccgccccggtagtgcccgaagaccaccagcaggtccgcgtagtgggcggcggtgatccacgacttgcggccggtcaccaccacctcgccgtcgccggtgtcggtgatggtcgtggtcatcgcggacaggtcgctgcccgcgcccggctcgctgaacccgaccgccgccagcccgcccgaggtgagcctgcgcaggaaccgctcgcgctgctcggcggtcccgagcctgcgcgcggtccacgccgccatgccctgggaggtcatgacgctgcgcagcgagccgcacagctcccccaccgacgcggtcagctcgccgttctcccggctgcccagcccgaggccgccgtgcggggcgccgacctgggcgcacagcacccccagcccgcccagctccaccagcagctcgcgcggcagctcgcccgccaggtcccacccggcggcgcggtcgccgacgcgctcggcgaccagcccggccagtgcgacggcgtcgctcaccgccccgcctcccgcagccgcagcaccagcgtggtcatggtgttgacggtgcggaagctgtccaggcccaggtccggcccgtcgatcacgacgtcgaaggtcgactccaggtgcaccacgagctccatcgcgaacatcgaggtgacggtgccggacgcgaacaggtcggtgtccggctcccaggtctgcttggtgcgctcggcgaggaacgcctgcacccgctcggccaccgcgtcggcggtgagcgcgccgggctgggaggaggtcgtcacagctgtgccttcccgtagtcgtagaagccccgcccggacttgcgcccgaggtgcccgtcgcggaccttgcgcagcagcagctcgcagggcgcggagcgggggtcgccggtgcgctcggccagcacgcgcagcgagtcggccaggttgtccaggccgatcaggtcggccgtgagcagcggtcccgtgcggtggccgaggcagtcctgcatgagggcgtccacggcctcgacggaggccgtgccctcctggacgacgcggatcgcgtcgttgatcatcgggtggacgatccggctggtcacgaagccggggccgtcgccgacgacgaccggtgtgcgggccagctcgcccagcacgcccacgagggtctccagcgcgtccgcgccggtgcgcgcgccccggacgacctcgaccgtggggatcaggtacggcgggttcatgaagtgcgtgccgatcagccgcgccgggtcggggacgtgcccggccagctcgtcgatcgggatcgaggaggtgttggacaccagcggcacgcgcggcccggtgagcgcggcggccccggccagcacctcggccttgaccggcagctcctcggtgaccgcctccaccaccagcgagacgtccgcgacgtcggcgagcgaggtggtggtgagcagctcgccccgctcgcggtcctcgggcagcgcccgcatcagcctggccatgcgcagctgggcggccaccgcctcccgcgcccgcccgaccttggcccggtcggtctcgaccagcaccaccggcacgccgtgcccgacggccagggaggtgatccccaggcccatcgtgcccgcgccgagaacggcgagcaccgtcctgccgtcctgctctcccatcgcgctcccccgccgcggccaccgcggccgccgtccggtccgcgcgccgtcccggcacgcgcattccaccctcgatcgtgtgccgggaaaggcgcgcccgaccccctgacctgcccccctgaacccccctcaacggaaccggaaatcgaatgtcccgaacgcgccgtcaaatcgtcgattgacagccgcagaactgttcatagactgtggcggcagtaccgatctccgaattccacggaagagtcctcccccatggctcagcagatcagcgccacctcggaaatcctcgactacgtccgcgcgacctcgttgcgcgacgacgacgtgctcgccggtctgcgggagcggaccgcggttctcccggccgcgtccgcgctgcaggtggccccggaggaggggcagctgctcggcctgctggtgcgcctggtcggcgcgcgctcggtgctggaggtcggcacctacaccgggtacagcacgctgtgcatggcccgcgccctcccgcccggcggacgtgtcgtgacctgcgacgtcgtcgcgaagtggccggacatgggcaggccgttctgggagcgggcgggcgtcgcggaccgcatcgacgtccgcgtcggcgacgcccgcgccaccctggccggcctgcacgccgagcacgccgtgttcgacctggtgttcatcgacgcgaacaagtcggattacgtccactactacgagcgcgcgctgacgctgctgcgcaccggcggcctggtcgtcgtggacaacacgctctttttcgggcgggtcgccgatccgtccgcgaccgatccggacaccaccgccgtgcgcgagctgaacgcgctgctgcacgccgacgagcgggtcgacatgtgcctgctgccgatcgcggacggaatcacgctcgccgtgaagcggtgaacccgcccgaatcgcgccgaattcccccggagagaaaggccgccgcagtgttcaccgaggacgtggccaccgacctgcccgcctacccgttcctgcgggaccggggcgactgcccgttcgcgccacccccgcgctacggccaattacgggaggagcagcccgtcaccagggtccgcctgtgggacggcagcaccccgttcctgctcaccggtcacgaggtgtgccgcaccgccctgaccgacccgcgcttcagctccgacggcgccaaccgcgcccagccgcgcttcgtgaagttcgacatcccggacgacgtgttcaacttcggcaagatggacgacccggagcacgcgaggctgcgccgcatggtcgccgggcacttcgcgagccgccccgtggaggcgatgcgccccgcgatcaccacgatctgccacgcccagctgcgccagctcgtgcaggcgggctcccccgccgacctggtggcccactacgcgttcccgatcccgtccctggtgatcggcggcgtgctcggcgtggcgggccccggcctggacgagttcgcgcgcgactcgacgcgcgccctggacccgtccctgtccgccgaggagatgggcgccgccatcaactcgatggtcgggttcgtggacgacctgtgcgcggccaagcgggccgcccccggcgacgacctgatcagccgcctggtgctggacttcgagcgcaccggcgagctgacccggaagcagctcgtcgccaccgtgatggtcgtgctgctggcgggctacgagaccaccgcgaacatgatcgcgctgggcacgaccgcgctcctgcgcgaccccgagcagctggccttcctgcgcgccgagcccgccggtttcgccaacgccgtcgaggagctgctgcgctggcacaccatcgtccaggacggcaccggccgcgtggccctggacgacgtcgagctggacggcgtgctcgttcccgcgggctccggcgtgatcgtcaacctgcccgcggccaaccgcgaccccgacgtcttccccgatcccgaccgcctcgacgtgaccaggcacaacgcccggcggcacttcgcgttcggctacggcgtccaccagtgcgtgggcatgacgctggcgcgcgtcgagctgcagatcgcgctggagaccctgctgtgcggcctgccgggcctggcgcctgccacgccgttcgaggacctggacttcgccctggagtccatgaacctcggcctgcgctcgctgccggtcacgtggtgagcaccgaccgtccaccaggggagagccgatgacccgcaccacccccacccccgacctggccccggagttcccgatgcccaggtcgcccgagcacccgttcgacccgccccctcgactccgcgaggcgcaggaggcgggcggcctgtcgcgggtgcgcctgtgggacggcagcaccccgtggctgatcaccaagcacgcccaccagcgcgagctgctgcgcgacccccgcctcagcgcggacttcctgcgccctggctaccccagcccgattcgcatcgaggacaagtcgacgttcatcagcagcttcccgctcatggacgaccccgagcacaaccggcagcgccggatggtcctgggcccgttcaccgtccgcaaggtggaacgcctgcgcccgttcgtgcagcggatcgtcgacgagaagatcgacgaactcctcgcgggccccaacccggtcgacctggtcaccgcgttcgcgctgcccatcccgtccctcgcgatcagcgccgtcctgggcctgccctactccgaccacgaggtcttcgagcgcaacagcgccgtgctgatccgccaggacgtgcccccgcaggaacgggccgaggccagcgaggagctccagcaccacctcgaccgcgtcctgggcgacaagatgaccgaccccgccgacgacctcctctccgacctgggcgcacgggtgctggcaggcgagatcagcaggccggaggcggtcgacatgaccgtcctggtgctggcgggcgggcacgagaccaccgcgaacatgatcgcgctcggcaccctcgcgctgctccggcaccccgaccagctggcgctgctccaggcgggcgacgaccccgccctcgccgagaccgccgtcgaggagctgatgcgctacctgacgatctcgcacaccgggatgcgccgcgtggcgaccgaggacgtggagatcgacggccaggtgatccgcgcgggcgagggcgtggtgctggcgacctcgatcggcaaccgcgaccccgacgtctacgacggcgacccgcacgtgctggacctgcgcaggccggtgaagcagcacttcgcgttcagcttcggcacccaccagtgcctgggccagtcgctggcccgcatggagctgcaggtcgtcgtgaacaccctctaccgccgcgtcccgaccttgcgactggcgaccgcgctggagcgcatcccgttcaagcacgacgggatcgtctacggcgtctacgagctgcccgtcacctggtgaccccgtcccaccagacctcctgccacgcagacctcccgcaagccgaccccgaaaggccgttcccatgagcgacaccacgctgtccgtgcccgtccccgaggaggtcggcaagctctacgaccagatcctgaaggacgagcacacctacgagcagttcgagaagttcaaccaccagctgcacatcggctactgggacgacccgacctcggacgtgcccatgcgcgaggccgtggtgcgcctgaccgagctgatggtcgagcgcctgcgggtggacgccgaggaccgcgtgctggacctgggctgcggcatcggcggcccggcgacccagatcgtgcgcaccaccggcgcacgcgtcgtcggcgtgagcatcagcgaggagcaggtcaagctcgccaccaggctggccaccgaggcgggcgtgggcgaccgcgccaccttccagcgcgccgacgccatgcggctgccgttcgaggacgagtccttcgacgcggtgatggccctggagtcgatcctgcacatgccgtccagggagcaggtcctgtccgaggcgcgccgggtcctgcgccccggaggccgcctggtcctcaccgacttcttcgaacgcgcaccccgcacgccggggatgcaccccgcgatcgagggcttctgccgaaccgcgatgacgacgatggccgacgtggacgactacgtgccgatgctgcaccgggtgggcctgcgcgtgcgggagctgctggacatcaccgagcagaccatggaacgcacttggcgggagaccctggagatcgtcagccagaacgaccgcccggtcgacttcgacctggcggagctgttcggcgtggacgagttcggctgcctgctggtcgccgcagaccgcccgtgaggcccgtccccgaggccgtgggccgcctgtacgacgacctgctggaggccgagctggaggggggcgcagccgacccgaacctgcacatcggctactgggacgcgccggactcgccaacgccacgcgcggaggcggtagtgcgcttcaccgacgaacacgtccgccgcctgcacgtgaccacgggcgaccgagtgctggacgtgggctgcggcgtaggcggcccagccctgcgcgcggtggacctgaccggcgcccacgtgaccggaatcagcatcagcgccgcccagatcacccacgcgacccacctggccaagtccgcgggccacgcggacaacaccaagttcctccacgcagacgcgatggccctcccgttcccggactcctcgttcgacgcggtcatggcgatcgagtccctgatccacatgcccgaccgcgagcgggtcctgaacgaggcaagacgcgtactgcgcccaggcgggcgactggtcctcaccgaactgttcgaacgcgccccaagacccacccgcagacacccagcgataaccgagttctgccgagcatcgatggtgtccctgcccaacgcagacgactaccccgcactactacaccgagcaggcctacgcctacgggaactcctggacatcaccgaccacaccgtccaacgcaacttccgcgaactggccgatctggtaggcgacgcgaagggcctgctgttccacccacgcgacctggtgggcgtcccagaattcggctgcttcctagcagtagccgaacacccgtaaccacgcggtggcgtcccccacggacgccaccgcctcgcgggctgcggggcgagcgcagcgagcccgcgcagccccactcccgcgtccctcttctccgtgtggcctggcgcatgtcaaattcccactgactgccaacagatcatgtgccgtttgagcaggtcagcgacttgtcgcgcttcggtgccttaaggccgagctgggatgggggcactgtttccggactgagcggggcagcttggaaggtggagttcggtgagcagaggcagcacgtcccgtcgcacgtagaggtggttgtacacgcggtggcgggacctgcgcagtaggccgctatccgcaagctgctccaagatcaggagtgcggcgcggtgcgtatagccgagttcggcggtcagcatggtgctgttgagcagtggggcgacgagcagcggggcgggaagcgctttgaccttcctccgcccggtgcgcatcgcccaggtgggcgatcgcgcgagcctcacggatcgcggtcacctcatgcaggctggcgctcaacctggaacgcgcgactgtttcgtccagacgtgccagggcggtgtaggcgtgcaacaaggtcttgctggtttcggagcgcagtctgagccgggaccaggacgacaactccgcgatcctcgcggacgggggcggcctcgtgtcttcaccggtggtagttgacctgcgcggggcggaggtgccctattgctgccgggacgaggtcatcccccggagcagtttctcagcacgccgtgaatcgagatccggggcgctgagcgcggtgaacgcctcgtccagcgagtcgcacgcgcacgtcgtcctgacatcgggccgcgcatggcccgaggtggtcagcggtgagcgggaaggcgcggcagggtgtgtgcgagacactccgggactccgtgcagaaggtcgatcaggcgaaagggttgaactgcgaatcgcaaagcggcccggccgcaaaggggtcgggccgcctgcgacgattggtcacgctgctgcggcgcggtcccgccggaactgcttgccgagcaggtcgatccgccccttgtgatcttctgccagcgcctccagaaccgagagcagtcgtcgggcgtgcagtgcatggccaataccatcgtcgcgtaccccagagggtgtcgctcccgttcaggggcgaccatttcccacgcccgcttggcctccttggcggcccggccaagatcgccgagcatcaggtaggtgcccgacaacccgacaaccctgcctgccaacgcggcttccggcaccccgcgcgcctcgtcggcttccaacgcccgaacaccgtgccacagcacggcccgcgcgttgccctcgctcgtctccagccatcccatgacaccgtgcgcttcggccagtgaccacgatcggctgtcgggatcggtgttgcacaacgccagctccagcgctcgttcagcagcgttaccgaccacagcgcggcgccgatgtccagcacttcttgccggtacccgcccacgagtgcggcggtgcgctgcacggccacgacttcccgccgatgcacgatcagccacttgtacgccgccaaggcgttgtcgaacagcggcttcgccccgacctcgaagccgtcgacgaacacctcgcgcaaatcaccgagcagtttccctgcggccaaggtgcgccgatgcaggtacctgcccaagcgctccaactcttgcgggaactcctgctgcacagcccatcgcagcagtgcctgggcttggtctgtctcctgcgcgcgatggcgaccggccagccggtaacgcgaggaggtgaactcggggtgcgtgatgttcgggtgaagttcagtccacgaaggctgcgtcagcaccagcacgccctggttcacccagtcccgcgcgtgttgggcggtctcctcgacggtgattcccagcatcgcggccaacgacgaggtcgagaccacggggtccggcagcaggtccagcaatctcagctcttgcggaatgggcacaagagtgttgatcatcgatgcccctcccggaggacggcgatgattggagtggcgaacagaaggggaaacgccagttcgccgggttccggcggtccacgcgccttcggccggccacttggactccgacgggcagaagttcaccggcaaggactctggtgacggtggagcggtgcacgcccatcgcttcggccaaggcgtagtcgctgtggtaaccagcgaactgagctagctttcgcatcttgtcgccgcgcaccccgacgaccttcttgatcttttcggtctcgctgtcgttgtcgtcgacatgtccgccgtccggcgctgacaccgttctccttgagatcgccgagctgaatgggggatgcttcgacgtaaggcgttgcgtatgcgcaacaggtcaggcggcgtcgagtctccccattaccgaggtttcgcttgatcgccgacggggcccgcctcgaagaagtccaatcgagctggcatccccttcgattgatcaatagcgcgacgggtgtcgctcgacatcgccccaccgcctgctcctgacgtgccacgagcagggaggagcgacctccctcgggactgcaccgaccgttcctccctgtccgccgattcagttgcattccgccacgctaggtgccggatgcgggccgaagggacaacgaagggacaagtcgaacagcccaggtgcgaggtatcttgaaatagcccgaatcctccgtcgcgaagcaggtcgccatgcccactgacgaacagtccgagggtgtcggagagcgcatagccgtccaacgcaaactggctggcttgactcagcaagctctggcgaagcgcgcacacgtcagcctcagcctcatcaaaggggtggaacagggaaggattcccgcctctcccgcgctcgtgtcccaggtctcgcgggcgctcaaggtcgaggcgacgatcttgctggggcagccgtaccgccccgaggatcggagcagtcttcgcgttcactccgtcatccccggtctgcgccgagccttggcggcctaccggttgcccgctgatgagggcatcagccctcgcgggtacgacgagctggccgccggtgtagccgccgcgtcgaagatgcgccacgccgcgacgttggacgtcctgggggctgaactccccggcctgctcgacgagatccgctcggccatcgacgaggctcggggagttgagcggcagcgcctgttcagcttgctggcagaggcatacgcagccgctggtcaagtcgcgtggaagctgggttacgcggacctgtcctccctggcgacggagcgcgtggagtgggcggccaaagagtccggcgatccgctcgcgatgggcgcagcggacttctacatcgccggtgagctgatcgcagcagcggagtggcgcggcgccctctcctacctcgacggctcccgtcgccgcctggagcacgtggtgcgcaaggacgacgaggccgccttgtcgatctacggagtcctgcacctgaagtcggggctcgcggcggcacgggccgggaaagccgacgaatccgacgcgcacctcgctgaagcccgtggcatcgcggaaagggtgccgctgggcagtgaccactaccggctcgcgttcgaccgggactcggtcaacatctggaccgtggggctggcagtggagcgcatggacggcacggaagccgtcaaacgagcccacgggatgcgcttcagcaagaccaccccgcgtgaacgcgtgggccaccactacatcgatctggcgcgcggctaccagctgcacggagaccgtgaccgcgccctgcacacccttcagatcgccaggcgaacctcaccgcagcaggtgcgctaccacccgcaggtcagggaaacccttctcacgctcgcggaacaggaccgcaggcgctcggattccctggcagggctcgcgcgctggatcggtatgccggtgtgacaggacggcgagctgacgtcgctgttgaggggcagccccccatcggccgcccctcaacagcaggtgccggtacgtccctcacagcgcgacgctgacgatcaggctggcgaacatggccacggccagcgcgatcatctgcttgggcgcgccgccgaagaagagggaccccacccaccgcagtggtaaaccggcaccgagcaccaacggccaccggccggcagagcccgtccccctcttttttggaggtctgccccgccggcgaggcgtgcccttcagctctcaagctctccactctcgatcttgtggtccgaacaccccccgcacccccactacgatccccccatgggggctctgatcatcgcggtagtgctgctgctcgtcttcctcgtgcaactcaagcgggaacccagacgactgggcaacggcgtctacctgctgatgagcctggcgttcttcgccctctggctgctcaccctcgccaccccccagaccaggacgctggtggtaggcgcggtagtcctgatcgccccggtattcgtcaccgtgatcgccctgttcctcatcgccaacggcgtcaccctgctgcgccgcgagggcgtcaaaccaggcaacgccctctccttcggcgcaggcaccgccatcctgtgcgtcgtaggcggcctgctcctggtcctgctctccgccctgcgcgaaggctcccccgacccctgggtgctggcagcagccggttccctggtcctcctggccggctacctgggcttcgccttcaccctcttcctgctctactccgtgctctacggccgagtccgcaagcgcaccggccacaccgcgatcatcgtcctgggcgcgggcgtccccggcggccgagtgaccccgctcctggcaggccgcctggaccgcgccctgaagctctaccgccgcgccgcagccaagggcgcttcccccgtggtagtcgcctctggcggccaaggcccagacgaaccagcctccgaagccgaggtcatggccaactacctccgcgaacgcggcatcccggacgaggccctcctggaagagcgcgagtccacctcgacctgggagaacctccgcctctcctccgccctgctcgccgaacgcggcgtgaccggcagactcctggtcgtcaccagcagctaccacgtcccccgagccgcgatcctctcccgccgcgcaggcctgaaggcagacgtccgcggcggccgaaccgcctggtacttcgtgccgaacgccttcctccgcgagttcgccgccctcctggtccagtaccgcaccctcaacgccctggcagcctgcaccgcactctccgtcttcccgctcctggcctacggcgtctgaaaagcacgacccggccgaccggacaccgcgtcacagatccagcggcgccgacccgaaggccacgttgaaccggtcgcaccacaccacgacgctgcgcagccccgacaggtccacgtcctccggaatcaggtagttctggttgccgtcggtggccttcatgggaccgagcggcaggtagcgcccatcgtcgtacttgccccactccccacccgcggtcgcgtcggagagccagatgtgcaggtcgggcccgtccgaggtggagaacccatccagccgcagcacgcgcgcggccccgctgcgcagcacggtggcggtgccccgcgtctcgtgctcctgggtgacgaacccacccgttgccagcaccgtcggctggtcggcggtcgccgacgacgtcccaccgggcgtcgtggcaccgctgcccgccgccgccccggtgctcgacgccccagccccggtgctcacccccgcaccggtcgagacgctgaactcggcgggcagcgcctcgtccgcctcgctgcgcgtccacaaccgccacggctggaacacccacagcccgacgaccgcagccaccaccacaacccccgacaccgcccaaaccgctctcctgcgcaccgaaccgcgcaccacgtcccctcccgttctccgcagacgacctgccaccatgccacgggtcgcgcccgatgaccacgaccaccgcgccacacccgccccacgcagcgactaggctgcccaccggggtcgccagccgatcccgagcgggttgagcaggcagcccaccgcagttcgcgctagtgggatggagggagcgggccggtgtccgagctggatgcagccgcggtggtcacggtgggttccgacgtggtgcgcggggtgcccgtgctgcgcgtcgccggggagatcgacaccaacgtcgccgacgaggtccgccgggcgctgctgccctggctggacgggttgcgcgggccaggggtgctcgacctgaccggggtgaggttcatggcctccaccgggttgtcgctgctgatcgaggccgcccggcgcaggccggcgaagctggtgctggccaccgcccagcgcggcgtgctccggccgctgcagctgaccgggatgagcgcgctgctcccgacgcaccccaccgtggacctggccgtggacgcccagctcggggccgccctggccgggatgcccagcacggcctgaccaccctcggtccacgggcggcctgcccgcggaccacccgcacggcgccctggggggacgagatcacagctggtggaagacgcgatcctggtccgcgcgccgcgacgccggtgggcgcgggcgctcccgccacggcggcggacccgcccccggtccccaccacggctccggcaccggccccgacaccgacaccgaccccagccccgcccctgggcacgaccacaccaccaaccccggtcctgggcgcaggtgtcgccaccgccaccgccctgacgctggcactcgccggggccgcagccccagccgacaacagcgcgggaaaggcggccatcatggacgaggtggacgccccaaccaccccacccaccccggccgcgctggacctcacgccccgcccacccctggccgaggtgcgccgctggaccggcgcgctgctgatcgacgccgacgaggaagcagcggacgacgtgctgctcgtggtcaacgagctggtcgccaacgcctacgaccacaccacctccccactcgccctgcgcctcaccaccacccccgagcacgtgcgcgtggaggtcgaggacggctcccccgacccaccacgcccggacctcaccgcgggcctgcgccagatcggcacgcgcggacgcggcctgctgctgatccgccagctgaccgatcgctggggcagcacgccccaccccggcggcaagaccgtgtgggcggagctgccgaacgtcccggcgacctgagcccgacgccccaccaacgaggccacggcggatctcacgggaagagcgcggcggggcactccgggcgcgttggacgccggcgcactccccggtgaggggtcgggcggcggagtggatgagcgtggcggcgagcagggccggtccggcggacgagacggccatcagcagcccgccgaccggggccgcgaggcgtcgggcgcgggcgccgagcctgcccggcaccgggccgaccacggagctgacgccgaggacggcggtgcaggcgccgagcgcgcgcctgcgggccgcgccctcgaagcgcacctggacggcggtcagcgccccggagaccatgagcgccgcgcccgcgccctggaccacccgcgcggccaccagcgccgggccggtgggggtgaggccgcaggccggggaggcggcggtgaaggtggcgggcccgacgaggtgggcccagcggcggccccggatctcgccgaggtgggccccggtgatcagcaggacggcgagctgcggcaggacaccgacacgggcacgaccgcgtcgatgtgcgtcgcggcggcgcagggcgcggagacgggcgcgggcgagcgctgagggcccgcccggcgccactcccccgtcacacctcccgccgcagcacatcctcctccgtctcccgccgcaccagcacccgcgccactccgtcgcgcacgcccaccacaggcggcctgcccacggcgttgtagttcgacgccagcgcgtggtggtaggcgcccgtcaccggcaccgccagcaggtcccccgcgcgcacgtccgcgggcagcggcacgtcctcggcgagcacgtcacccgcctcgcagtgcctgcccaccaccgtcaccggcgcgcgccgcccgccccggccgaccaggcgcaccgcgtaccggctcccgtacagcgcgggcctggggttgtcgctcatgcccccgtccacggccacgaacacccgcctcaccccgcgcttgacggcagccacccggtacagcgtcacaccagcgcccgcgacgaccgaccgccccggctcgatcagcagcctcggcaccggcacgcgccgcagcgcgcactcgtggctcagcgccacccgcacccggtgcgcgaacccgccaaggtcgaactccccctcccccggcaggtagggcaccgcgaacccgccgccgaggtccagctgctcgatccgcaccccgcacgaggcgatcagcccgaccatccgccgcgccgcctcctcgtacaccgcgacgtgtcgcacctgcgacccgacgtggcagtgcagccccaccagcctcagcgacggctgctcgaccacccgcagcaccgcctccagcgcgtccccacccgccagggagaagccgaacttctggtcctccaccccggtcgccaccgcccggtgggtgcgcgggtcgacgccgggggtgacccggaccagcacgtcctgcggccccctggccagcgcgcccagctgctcgatctcgtcgaacgagtccaccaccacccgcccgaccccgtacccgagggcggccttgaggtcctcgggcgtcttgacgttgccgtgcagcagaatccgctccgccgggaacccgaccgaccgcgcgatcgccagctcccccgccgagcacacgtccagcgacagcccctcgtccgccacccaccggtacacctcgcggcacggcagcgccttgcccgcgaacaccacctcagcctccggcagcacctcccggaacccgcgcgcccgcgcccggaccgtgccctcgtcgagcacctggcagggcgtgccgaaccgggcggcgagctcggtcgcgggcaccccgccgagcagcagctccccccgctccagccgggtccccaggggccacagccccgcctccagggccggttcgccggtcatgccgacgctgggcagcaactccgcgagtgtcatgcccgccagcacacgcccgaaccggccggggcgacagcggcgcgaacgcgtccctgacggcgtgccgggcgggattgacgccgccctgacccgaccgccccagcccgctctcgaacccggcggaagcacccccgaaacgcgccggaaacccgcccgcgcattcccccgaacgcctacctcacggcgattttgatgctttttttacgccgggacgccgcgatattcactcctccgagccgcgcggggacgttgacttctcatgcccgacgacgtgatcgaggagagaccccgaatgtccgaaacaccggttttcgccgttccacccagggtggaaagcccggtacgcccggccgcgcccgccaaccgggtggggcgctggctgctggagcaccgggtgcaaccggcgggacccgcgggcaccgaccagcacagcacgccccaggcgtggtggaaggtcatgtgcctgaccggcgtcgactacttctcgaccctgtcctacctgccgggcatcgcggcgctggcggccggggcggtctcgccgctggcgacgctgctgatcgtcgcgctgaccctgttcgggatgctgccgatgtaccgccgggtggcgcacgagtcgccgcacgggcagggctcggtggcgatgctggaggacctgctgccgttctggcgcggcaagctgttcgtgctggtgctgctgggtttcgtggccacctcgtggatcatcacgatcaccctgtcggcggccgacgcgtcggtgcacgcgctggagaacccgcacgcgcccgcgttcctgcacgggcacgaggtgctggtcaccgtggtgctgctgctcgtgctgggcggggtgttcctgctgggcttcaccgaggcggtcagcgtggccatcccgctggtcgcggtgttcctgctgctcaacgcggtggtcgtggtcgccggcgtgctggaggtgatcgcgaacccggacgtgctggacggctggttcgcggcgctgacctccaccggcggcggcggggtgctgggcgtggtcggcccggccctgctggcgttcccgctgctcgtgctcggcctgtccgggttcgagaccggggtgagcatgatgccgctggtcgaggcgaagggcgccgacgacgccgaacgcctggcgaaccgcgtccgcaacacccgcaagctgctcaccaccgccgcgctgatcatgtcggtgtacctggtggccaccagcttcgtgaccaccctgctcgtgccggtcgagcagttccgccccggcggcgaggccaacgggcgggcgctggcctacctggcgcacgagctgctcggcgagtgggtcggcacggcctacgacatcagcagcgtgctgatcctgtggttcgccggcgcgtccgcgatggccgggctgatcaacatcgtgccgcgctacctgcccgcgtacggcatggccccggactggacgcgcgccgtccgaccggtcgtgctggtctacacggtgatctgcgtcggcatcacggtgatcttccaggccgacgtggacgcccaggccggcgcgtacgcgaccggcatcctggcgatgatggtgtcggcgtcggtggcggtgaccctgtcggtggcgcgcgccgggcggcggggcgcggcctcggcgttcgcggtgctgaccctgatcctggtgtacgcgctggtggagaacgtgatcgagaagccggacggcatcacgatctcgttcgtgttcatcgtcggcatcatcgccgtctcgctggtctcgcggatctcgcgcaccaccgagctgcgcgtggagcacatcgagttcgacgagaccgcgcgcaggctcatcaccgactcgatcgcccacgacggcgcgctgaccgtgatcgcgaaccgcaggcaggccggtgacgtggccgagtacgcggacaaggaggccgagcagcgcggggtgaacccggtgccggggcaggcggacgtgctgttcctggagatcgacgtggtggacccgtcggacttcagcgacgtgctggaggtgcgcggcgtggaggtgggcggccaccgggtgctgcgcgcggacagcccggcggcgccgaacgcgatcgccgcgatactgctggcgctgcgcgactgcaccggggtgcgcccgcactgccacttcgcgtggagcgagggcagcccgctggggcacctgttccgctacctgctggtggggcgcggcgacacggcgccggtggtgcgggagatcatccgggcgcacgagtccgacccggagcgcaggccgggcatccacgtgggggcctgagcgggcacgacggcggggtggtccaggcaggcagcgtggtccaggccagtggggtgctcccggccagcaacgtgctcccggccggtgggggctccagggcgctgcggcggccgatcgcgcgggcgtggtcggcgaaccgctcgcagtgctcgctgagcagggccgcgtcgacggcggcgtcctcaacgccgcgcagcacggccagcacggaccggggcactcaccaaacgcgaagagccacaccaactgggcttcggcgtgggaggcgcggtgcagcggtttgtggtctcgcgctgccgcgcggcgcgggggactgggtcgcgagcagcacctggccgccgtgccgcgcggcgccccgcgccaggtcgcacacggcggccaggtccggcaccggcgcgtcccgccggtcgtggaacacgtcgcgcatcgcgctctccctcggaggatcggatcggaaggccctgatcccaaccgggcgcgcaccccggcgacaagccctcacccgccgaacttgcgctttccttccgccccgacccccgcccgtcacaaacccccgtcaccccgccgtcactttttgtgatgacgatcaggaaacagtagtagcccattcgtgacctgcactgacgcgcagatcaccccacccgtcaacgaaacgtaaaaccgcctggtcaccccgtcaaagacccgtcagcaccccgctcacggcgttttccccgttgcacccttttggcgtcgcggtccccacgaacgggggccgctcggagtcgggaagggagcacgctcatggccgacctggcctacgcgtcgctgctcatcgctgtgttcggactgctcgtcctcggcattcgcggactggggcggctctgatgggcggcacgggagtcgtggccaacgccgtcggtggcgtgctggccctgctgctcatcgggtacctgttcgtcgcgctgatcaggccggagaagttctgatgtcctcgaccacggcgggcctgctccaggtcgccctgctcatcgccgcgctggccgccgcctaccggccgttcggcgactacatggcccgcgtctacaccgacgccaagcacaccaaggtcgagcgcctgctctaccgcgcagcccgcgtcgaccccgactcgcagcagcgctggggcacctacgcgcagggcgtgctcggcttctccctcgtcggcgtggccctgctgtacctgatgcagcgagtgcagccctggctgccgttcgaccacgaccggggcgcggtctcgcccggcatggcgttcaacaccgccgcctcgttcgtggccaacacgaactggcagtcctacgtcccggagaccgtcctcggccacaccgtgcagatggccgggctgaccgtgcagaacttcgtctccggcgcggtcggcatggccgtcgccgtggcgctggtgcgcggcttcacccgcgagggctccgaccggctcggcaacttctgggtcgacctcaccaggggcaccctgcgcgtcctgctgcccgtgtcgttcgtgttcgccatcgtgctggtcgcgaccggcgtcgtgatgagtctgaaggcgggcgtggacgtggacggccagcaggtcgccatcgccccggccgcctcgcaggaggccatcaaggagctcggcaccaacggcggcggcatcttcaacgccaactccgcccacccgttcgagaaccccaacggctggtcgaacctggtcgagatcttcctgatcctgctgatcccggtctcgctcacccgcaccttcggcaccctggtcggcaaccgcaagcagggctacgtgctgctcagcgtcatgggcgtgctgtggaccgcgatgctcgcggtcatctgggcggccgaggcgcacggcctgcgccccctggagggcaaggagctgcggttcggcgtccccggcagcgccctgttcgccaacaccaccaccgccacctccaccggcgcggtcaacgccatgcacgacagcctcaccggcctgggcggcggcgcgacgctgctgaacatgctgttcggcgagatgacgccgggcggcgtcggcaccggcctgtacagcatcctggtgatggcgatcatcgcgatgttcctggccggtctgatggtcgggcgcaccccggagtacctgggcaagaagctgggccgccgcgaggtgacctgcgccgcgctgtccatcctggcgatgcccgcgctggtgctggtcggcgccgggatctcggcggtgctgccgtcgacggccgggtacctgaacaaccccggcgagcacggcctgtccgagatcctctacgcctacgcgtcggcctcgaacaacaacggcagcgcgttcgcgggcatcaccgtgaccagcgactggttccagtcctcgctcggcgtctgcatgttgctcggccggttcgtcccgatcatcgcggtgctgtgcctggccggttcgctcgcccggcagaagcgcgccccgcggaccgcgggcacgctgcccacggacagcccgctgttcgcctcgctgctggtcggcgcgatcgtgctcgtcgccgccctcaccttcgtccccgccctcgccctcggccccatcgcggaggcactgctgtgaccaccaccgacacccgccagcccgcccccgaggacacgggcgcgcggcccccggccaagcccgtcccgtcgggcgtgttcgccccgcgccagctgctcacgtccctgccggacgcgctgcgcaagctccacccccgccaccagctgcgcaaccccgtgatgttcgtggtgtgggcgggctcggtcctggtcacggtcttcgccgtcaccgacccgaacccgttcacgatcgcggtcgcgctgtggctgtggttcaccgccctgttcgccaacctcgccgaggccgtcgccgaggggcgcggcaaggcgcaggccgagtcgctgcgcaggactaagaccgacgcgctggcccgcctgaccgacggccgcaccgtgcccggcaccgagctgaaggtcggcgacctggtcgtggtcgaggccggtgaggtgatccccggcgacggcgacgtggtcgagggcatcgccaccgtcgacgagtcggcgatcaccggcgagtccgcgcccgtggtgcgcgagtccggcggcgaccggtgcgcggtcaccggcggcaccaccgtgctgtcggaccggatcgtcgtgcgcgtcaccagcaagccgggcgagacgttcgtggaccggatgatcgcgctggtcgagggcgcgcagcggcagaagacgccgaacgagatcgcgctgacgatcctgctgtccacgctcacgatcatcttcctgctcgcggtgctcgcgctccagccgttcgcggtgtactccggcggcgagcagtcggtgatcgtgctgaccgcgctgctggtgtgcctgatccccaccacgatcggcgcgctgctgtccgcgatcggcatcgcgggcatggaccgcctggtgcagcgcaacgtgctggccacctcgggccgcgccgtcgaggcggccggtgacgtggacacgctgctgctggacaagaccggcaccatcacctggggcaaccgccgcgccaccgagctgatccccgcgcccggcgtcacgctggacgagctggtggacgccgcccggttgtcgtcgctggccgacggcacccccgagggccgcagcgtggtcgagctgtgcgcgaccgggcacggccgctcccccgagcccaccgacgcggagaagaccggcgagttcgtgccgttcaccgcccagacccggatgagcggcatcgacctggacggccgcagcgtccgcaagggcgccgcgaccgcgttcaccctcaccgactcggtcaagtccacggtggacgagatcagcggcgacggcggcaccccgctggtggtcgccgacggcgagcgggtgctcggcgtgatccggctgtccgacgtggtcaagcccggcatgaaggagcggttcgccgagctgcgcgccatgggcatccgcacggtcatggtcaccggcgacaacccgctgaccgccagggcgatcgcggccgaggcgggggtcgacgactacctcgccgaggccaagcccgaggacaagatggccctgatccgcaaggagcaggagggcggcaagctggtcgcgatgaccggcgacggcaccaacgacgcgccggcgctggcccagtccgacgtgggcgtggccatgaacaccggcacctcggccgccaaggaggccgggaacatggtggacctggactccgaccccaccaagctcatcgagatcgtggagatcggcaagcagctgctgatcacgcggggcgcgctgacgacgttctcggtcgccaacgacctggcgaagtacttcgcgatcctgcccgccatgttcgccgcgatccacccgcagctggacaagctcaacgtcatgggcctggccacgccgcagtcggcgatcctgtcggcggtcatcttcaacgcgctgatcatcgtggtgctgatcccgctggcgctgcgcggcgtgcgctacaagccctccagcgcgagctcgctgctgcggcgcaacctgctggtgtacggcgtcggcggcatcatcacgccgttcgtcggcatctggctcatcgacctgctcgtccgcctcatccccggaatcgggtgaactccgtgaacgcgttcgtgaagcaggccctggccggtctgcgcgtcctgctggtgctgaccgtcatcaccggcgtgctctaccccgccgccgtctggctcgtctcgcgggtgcccggcctgcacgccaacgccgaggccaccggcaccgagctggtcgtggcgccgcgcgagggcgacggctggttccagccgcgcccgtcgatggcgacgctgcccgcgtcgggcgggtccaacaagggcgagcgcaacgccgactacgacgcggtgatcgccgagcgccgcaccgagatcgcccggcgcgagggcgttgcggaggacgccgtgccgcaggacgcggtgaccgcctcggcctccgggctggacccgctgatcagcgccgagtacgcggcgatccaggtgccgcgcgtggcgcgggagcgcggggtgtcggaggacgccgtgcgggcgctggtcgccgaggcgtcggtgggccgctcgctcgggttcgtgggcgagccgggcgtcaacgtcaccgccctcaaccgggccgtcgacgcggcggagtgagaccgaccgggggccgtcctcgcggcggcccccggtcttccccatttctctgatctcgggagcgggcgggaccgtggacaagcgcaagcgcggcgaactgcgcatctacctgggcgcggcgccgggcgtcggcaagaccttcgcgatgctcggcgaggcgcaccgccgccgggggcgcggcgcggacgtcgtcgtcgccctggtcgagacgcacggccgcgagcgcaccgccaccatggtcgacggcctggaggtgctgccccgcaaggaggtccagcaccgggggaccacgatcaccgagatggacgtggacgcggtgctggcccgcgcgcccgagatcgccgtggtggacgagctggcgcacaccaacgcccccggctcccgcaacgccaagcgctggcaggacgtcgaggagctgctggacgccggcatcgacgtgctgtccacgctcaacatccagcacctggagtcgctcaacgacgtggtgcgccgcatcacccgcgtcgagcagcgcgagaccatccccgacgaggtggtgcgccgcgccgagcaggtggagctggtcgacctgaccccggaggcgctgcgccgccgcctggcgcacggcaacgtctacgccgcgcacaagatcgacgccgcgctgggcaactacttccgggtcgggaacctgaccgcgctgcgcgagctggcgctgctgtgggtggccgaccaggtggacgtggcgctccagcggtaccgcaccgagcagcgcatcaccgacacctgggaggcccgcgagcgggtcgtggtcgcggtgaccggcggcgcggagagcgagaccctgatccgcagggcccgccgcatcgccgcgcgcgccggggcggagctgctggtggtgcacaccatgcgcggcgacggcctcgcgggttccgcgccggagtcgatccggacccgcgtcgggctcaggtgctcgacggtgctcttcaacgtggtctcctcgtaacgggacgtgcggaacaccccgcagcgcccagggtcgggcggctgacgggattcgcctgagtctaggcgaggccgcccccggccggggtggcaccccgcgaccgggtggttcacgtgcgggtgcgcgcgcccggcgcggcgcgcgcggtgcgagaggtgggccgtccggcggcgcgcggttttccgacatggcgcgcgcacgaaatagttttcggcgggtcgggcgccgtcgaatcgactcggggtcgggttttccgcgccaccccggaagcggacgaaccgggcgggcgaaccgggcgggcggtgcgcggacaacgggcgcgaccgccgcggtgcgccggtttgggcagcctttaccgccctccaggtcacccattccgccgttgcggggaacatccgcgtaccagtggcccccggcggacacgcggcccagcacccgctaggccgttcgcaggacgtcgtggtgcaccgggagcgtgaaaccgaacgtaaccggacagcggcgggctcaagtggggtaacactggcgccgcagcgcactcttacccacagcgacgaacgcggcggaacgctaccctttacaggtgaagtgaggccattcggagcaccggtgcgcagaaaactttcacgcccggagatgactccactcgccgtagtccattagtgtgggattccggtaccgttgcgccgcaggccgcaagaaggcggccaggaaagacgattaactcatccgggcgccccgccgtcgtgcacgtgaacgcgacgggcgaccgggaacggaacgagcgagacatgtcatcgcgctctttaccacctaccagaaaaggtgccgatgaccccgatgaagaccattccgccgattcccccgaacacgcgggcgtccgcccgtccgccgctcgggcaaccgcacgacgggcttgcgcgccacccggaaccccagggcccggccgcgaggcgatgttgacccgacccccggccaccagccgggacaggccgaccaccgccacgcgcggaacccacggcgaaccgcctctcgccgtgatccaccacggaccgttggggagttccatggagacccgtcaacttctggcgttcaccacagtggtgcagaccggcagcttcacgaaggccgccgccacgctgaactgctctcagcccacgatcaccaccaggatcaaggcgctggaggagaccctcggcgtcgccctgttccgcaggttgccgcgcggcatccagatgacctccgccggggtcgagctgctgccgttcgcgcgcaacatcatcacgctcaccgacaaggcccgcaaggcgatcaccatgaacggggagccgcacgggcacctcgtgataggcagcgcccagagcctcaccgactaccggctcttacccctgatcgagtacatgtgctggcgctacccgagcgtccagatctcgctgcactcgcgaacaacccggtcgaacctggccgccgtgcgcgagggcaggttggactgcgcgttcttcatcggcccggtcgagcagcgggacggtctggagacgacggtgctgtgccccgaaccgctggtgatggtcgcgggccccggccacgcgctggcgcggtcgggcgcggtcaccgaggcggacctgcggggcagcacgctggtcagggccgagaacggggcgagctaccacgagcagttcgagcgggcgctcgggctgcacgaggccgagtcgcgatcgccggtgctggccctggactcggtcgacgcggccaagcgggcggtcgcctcggggctgggcatctcgctggtgccggaggtcacggtcgccgcggagctggcggacggcaggctcagccgcatcggctggaccccgccgttccgggtgttcacccagttcgcgtggcgccaggacaactcggcgaacccgtcggtgaccgcgctggtctcggcggcggcgcaggtggtgagcgagcaggtggccgcgacacccgcgtagggcgtcgacgtgcagggtcgtggatgcggagcggccccctcgtgctgcgcagagggggccgagaccgtcggggcgacaggatttgaacctgcgaccccccgctcccaaagcgggtgcgctaccaaactgcgccacgccccggtcaccaggagcttagcgcgacgcgctaagctgttttcagcacccacccggtgggcgctgcgcgggtgtagctcaatggtagagccccagccttccaagctggtcatgcgggttcgattcccgtcacccgctccaccagatcc

SEQ ID NO: 12 - DNA, 27 bp, Artificial
  Primer
ccscgggcgnycngsttcgacngygag

SEQ ID NO: 13 - DNA, 28 bp, Artificial
  Primer
cgtcncggannccggagcacatgccctg

SEQ ID NO: 14 - DNA, 47 bp, Artificial
  Primer
atatactagtcacgtcaccggcgcggtgtccgcggacttcgtcaacg

SEQ ID NO: 15 - DNA, 42 bp, Artificial
  Primer
atatcctaggctggtggcggacctgcgcgcgcggttggggtg

SEQ ID NO: 16 - DNA, 42 bp, Artificial
  Primer
atatcctaggcaccacgtcgtgctcgacctcgcccgccacgc

SEQ ID NO: 17 - DNA, 42 bp, Artificial
  Primer
atattctagacgctgttcgacgcgggcgcggtcaccacgggc

SEQ ID NO: 18 - DNA, 30 bp, Artificial
  primer
ggtcactggccgaagcgcacggtgtcatgg

SEQ ID NO: 19 - DNA, 36 bp, Artificial
  primer
cctaggcgactaccccgcactactacaccgagcagg

SEQ ID NO: 20 - DNA, 30 bp, Artificial
  primer
cctaggaacgggtaggcgggcaggtcggtg

SEQ ID NO: 21 - DNA, 31 bp, Artificial
  primer
gtgtgcgggccagctcgcccagcacgcccac